SEQUENCE LISTING (267 sequences)

SEQ ID NO: 1 - DNA, 21 bp, Artificial
  siRNA
guuugauugugccauacuutt

SEQ ID NO: 2 - DNA, 21 bp, Artificial
  siRNA
aaguauggcacaaucaaactt

SEQ ID NO: 3 - DNA, 21 bp, Artificial
  siRNA
caugucuggcuugcaccuatt

SEQ ID NO: 4 - DNA, 21 bp, Artificial
  siRNA
uaggugcaagccagacaugtt

SEQ ID NO: 5 - DNA, 21 bp, Artificial
  siRNA
gauugagaagguucuuauutt

SEQ ID NO: 6 - DNA, 21 bp, Artificial
  siRNA
aauaagaaccuucucaauctt

SEQ ID NO: 7 - DNA, 21 bp, Artificial
  siRNA
gugugaagaagaaagcucatt

SEQ ID NO: 8 - DNA, 21 bp, Artificial
  siRNA
ugagcuuucuucuucacactt

SEQ ID NO: 9 - DNA, 21 bp, Artificial
  siRNA
gaacaaggaugcuuugcuutt

SEQ ID NO: 10 - DNA, 21 bp, Artificial
  siRNA
aagcaaagcauccuuguuctt

SEQ ID NO: 11 - DNA, 21 bp, Artificial
  siRNA
gaaaugaagccaucacguatt

SEQ ID NO: 12 - DNA, 21 bp, Artificial
  siRNA
uacgugauggcuucauuuctt

SEQ ID NO: 13 - DNA, 21 bp, Artificial
  siRNA
cccucuaugccaacaaugutt

SEQ ID NO: 14 - DNA, 21 bp, Artificial
  siRNA
acauuguuggcauagagggtt

SEQ ID NO: 15 - DNA, 21 bp, Artificial
  siRNA
ggguuugguggacuuccgatt

SEQ ID NO: 16 - DNA, 21 bp, Artificial
  siRNA
ucggaaguccaccaaaccctt

SEQ ID NO: 17 - DNA, 21 bp, Artificial
  siRNA
ccaacaauguucagagggutt

SEQ ID NO: 18 - DNA, 21 bp, Artificial
  siRNA
acccucugaacauuguuggtt

SEQ ID NO: 19 - DNA, 21 bp, Artificial
  siRNA
caagcuaaagaucaguauatt

SEQ ID NO: 20 - DNA, 21 bp, Artificial
  siRNA
uauacugaucuuuagcuugtt

SEQ ID NO: 21 - DNA, 21 bp, Artificial
  siRNA
gugugaauggaguuguccatt

SEQ ID NO: 22 - DNA, 21 bp, Artificial
  siRNA
uggacaacuccauucacactt

SEQ ID NO: 23 - DNA, 21 bp, Artificial
  siRNA
gucucaugauaggcauagatt

SEQ ID NO: 24 - DNA, 21 bp, Artificial
  siRNA
ucuaugccuaucaugagactt

SEQ ID NO: 25 - DNA, 21 bp, Artificial
  siRNA
caccuaugugcuucacugatt

SEQ ID NO: 26 - DNA, 21 bp, Artificial
  siRNA
ucagugaagcacauaggugtt

SEQ ID NO: 27 - DNA, 21 bp, Artificial
  siRNA
ggaauugucaguuuagauutt

SEQ ID NO: 28 - DNA, 21 bp, Artificial
  siRNA
aaucuaaacugacaauucctt

SEQ ID NO: 29 - DNA, 21 bp, Artificial
  siRNA
gguuaauagcucuauuauatt

SEQ ID NO: 30 - DNA, 21 bp, Artificial
  siRNA
uauaauagagcuauuaacctt

SEQ ID NO: 31 - DNA, 21 bp, Artificial
  siRNA
caacugcgaguacaucaaatt

SEQ ID NO: 32 - DNA, 21 bp, Artificial
  siRNA
uuugauguacucgcaguugtt

SEQ ID NO: 33 - DNA, 21 bp, Artificial
  siRNA
ccaaccagguguaugcagatt

SEQ ID NO: 34 - DNA, 21 bp, Artificial
  siRNA
ucugcauacaccugguuggtt

SEQ ID NO: 35 - DNA, 21 bp, Artificial
  siRNA
caagucugggccuugccautt

SEQ ID NO: 36 - DNA, 21 bp, Artificial
  siRNA
auggcaaggcccagacuugtt

SEQ ID NO: 37 - DNA, 21 bp, Artificial
  siRNA
gccauuaugagugugcauutt

SEQ ID NO: 38 - DNA, 21 bp, Artificial
  siRNA
aaugcacacucauaauggctt

SEQ ID NO: 39 - DNA, 21 bp, Artificial
  siRNA
gccaaaugggcagcccgaatt

SEQ ID NO: 40 - DNA, 21 bp, Artificial
  siRNA
uucgggcugcccauuuggctt

SEQ ID NO: 41 - DNA, 21 bp, Artificial
  siRNA
cucaaaucuuucucccuautt

SEQ ID NO: 42 - DNA, 21 bp, Artificial
  siRNA
auagggagaaagauuugagtt

SEQ ID NO: 43 - DNA, 21 bp, Artificial
  siRNA
guaaccuccuggaucugaatt

SEQ ID NO: 44 - DNA, 21 bp, Artificial
  siRNA
uucagauccaggagguuactt

SEQ ID NO: 45 - DNA, 21 bp, Artificial
  siRNA
ccaguaaccuccuggaucutt

SEQ ID NO: 46 - DNA, 21 bp, Artificial
  siRNA
agauccaggagguuacuggtt

SEQ ID NO: 47 - DNA, 21 bp, Artificial
  siRNA
ccuaugacuugagcagugutt

SEQ ID NO: 48 - DNA, 21 bp, Artificial
  siRNA
acacugcucaagucauaggtt

SEQ ID NO: 49 - DNA, 21 bp, Artificial
  siRNA
guaauuaaagaaaugguuatt

SEQ ID NO: 50 - DNA, 21 bp, Artificial
  siRNA
uaaccauuucuuuaauuactt

SEQ ID NO: 51 - DNA, 21 bp, Artificial
  siRNA
gcuaucaacuagaucgacatt

SEQ ID NO: 52 - DNA, 21 bp, Artificial
  siRNA
ugucgaucuaguugauagctt

SEQ ID NO: 53 - DNA, 21 bp, Artificial
  siRNA
gauaauaaauucaacuauutt

SEQ ID NO: 54 - DNA, 21 bp, Artificial
  siRNA
aauaguugaauuuauuauctt

SEQ ID NO: 55 - DNA, 21 bp, Artificial
  siRNA
gcuacaacuuacagugucatt

SEQ ID NO: 56 - DNA, 21 bp, Artificial
  siRNA
ugacacuguaaguuguagctt

SEQ ID NO: 57 - DNA, 21 bp, Artificial
  siRNA
caaaguuucuuuggaccaatt

SEQ ID NO: 58 - DNA, 21 bp, Artificial
  siRNA
uugguccaaagaaacuuugtt

SEQ ID NO: 59 - DNA, 21 bp, Artificial
  siRNA
cguuagucauccucuucuutt

SEQ ID NO: 60 - DNA, 21 bp, Artificial
  siRNA
aagaagaggaugacuaacgtt

SEQ ID NO: 61 - DNA, 21 bp, Artificial
  siRNA
ggaguauugggcaaccucatt

SEQ ID NO: 62 - DNA, 21 bp, Artificial
  siRNA
ugagguugcccaauacucctt

SEQ ID NO: 63 - DNA, 21 bp, Artificial
  siRNA
gaaugauuagguugccuuatt

SEQ ID NO: 64 - DNA, 21 bp, Artificial
  siRNA
uaaggcaaccuaaucauuctt

SEQ ID NO: 65 - DNA, 21 bp, Artificial
  siRNA
cacuuauucugguccuucatt

SEQ ID NO: 66 - DNA, 21 bp, Artificial
  siRNA
ugaaggaccagaauaagugtt

SEQ ID NO: 67 - DNA, 21 bp, Artificial
  siRNA
ggcuuaugcauuugugcuatt

SEQ ID NO: 68 - DNA, 21 bp, Artificial
  siRNA
uagcacaaaugcauaagcctt

SEQ ID NO: 69 - DNA, 21 bp, Artificial
  siRNA
caauggaccugucagcaaatt

SEQ ID NO: 70 - DNA, 21 bp, Artificial
  siRNA
uuugcugacagguccauugtt

SEQ ID NO: 71 - DNA, 21 bp, Artificial
  siRNA
caugucaguguugacuuuatt

SEQ ID NO: 72 - DNA, 21 bp, Artificial
  siRNA
uaaagucaacacugacaugtt

SEQ ID NO: 73 - DNA, 21 bp, Artificial
  siRNA
cuaacaagguggaccaccatt

SEQ ID NO: 74 - DNA, 21 bp, Artificial
  siRNA
uggugguccaccuuguuagtt

SEQ ID NO: 75 - DNA, 21 bp, Artificial
  siRNA
cuaaccaucccugagaucatt

SEQ ID NO: 76 - DNA, 21 bp, Artificial
  siRNA
ugaucucagggaugguuagtt

SEQ ID NO: 77 - DNA, 21 bp, Artificial
  siRNA
gccuauagucucagaguuatt

SEQ ID NO: 78 - DNA, 21 bp, Artificial
  siRNA
uaacucugagacuauaggctt

SEQ ID NO: 79 - DNA, 21 bp, Artificial
  siRNA
gagacauccugauaguugutt

SEQ ID NO: 80 - DNA, 21 bp, Artificial
  siRNA
acaacuaucaggaugucuctt

SEQ ID NO: 81 - DNA, 21 bp, Artificial
  siRNA
caaauggcuuuccaugaautt

SEQ ID NO: 82 - DNA, 21 bp, Artificial
  siRNA
auucauggaaagccauuugtt

SEQ ID NO: 83 - DNA, 21 bp, Artificial
  siRNA
cauuaaaguuaacauucgutt

SEQ ID NO: 84 - DNA, 21 bp, Artificial
  siRNA
acgaauguuaacuuuaaugtt

SEQ ID NO: 85 - DNA, 21 bp, Artificial
  siRNA
cacucaugacugaggucautt

SEQ ID NO: 86 - DNA, 21 bp, Artificial
  siRNA
augaccucagucaugagugtt

SEQ ID NO: 87 - DNA, 21 bp, Artificial
  siRNA
ccugucuugugugaggugutt

SEQ ID NO: 88 - DNA, 21 bp, Artificial
  siRNA
acaccucacacaagacaggtt

SEQ ID NO: 89 - DNA, 21 bp, Artificial
  siRNA
ccagcauucaccaaugagutt

SEQ ID NO: 90 - DNA, 21 bp, Artificial
  siRNA
acucauuggugaaugcuggtt

SEQ ID NO: 91 - DNA, 21 bp, Artificial
  siRNA
guuauuagaauguuacgaatt

SEQ ID NO: 92 - DNA, 21 bp, Artificial
  siRNA
uucguaacauucuaauaactt

SEQ ID NO: 93 - DNA, 21 bp, Artificial
  siRNA
gaguguagcaagagguguutt

SEQ ID NO: 94 - DNA, 21 bp, Artificial
  siRNA
aacaccucuugcuacacuctt

SEQ ID NO: 95 - DNA, 21 bp, Artificial
  siRNA
gcauguuugccaccaaugutt

SEQ ID NO: 96 - DNA, 21 bp, Artificial
  siRNA
acauugguggcaaacaugctt

SEQ ID NO: 97 - DNA, 21 bp, Artificial
  siRNA
gacgucuuugcauaugugutt

SEQ ID NO: 98 - DNA, 21 bp, Artificial
  siRNA
acacauaugcaaagacguctt

SEQ ID NO: 99 - DNA, 21 bp, Artificial
  siRNA
cuguucgagcggacguucatt

SEQ ID NO: 100 - DNA, 21 bp, Artificial
  siRNA
ugaacguccgcucgaacagtt

SEQ ID NO: 101 - DNA, 21 bp, Artificial
  siRNA
caguaccuguucgagcggatt

SEQ ID NO: 102 - DNA, 21 bp, Artificial
  siRNA
uccgcucgaacagguacugtt

SEQ ID NO: 103 - DNA, 21 bp, Artificial
  siRNA
gccauuuacccaguauaautt

SEQ ID NO: 104 - DNA, 21 bp, Artificial
  siRNA
auuauacuggguaaauggctt

SEQ ID NO: 105 - DNA, 21 bp, Artificial
  siRNA
gguaucaguuuaugaggcatt

SEQ ID NO: 106 - DNA, 21 bp, Artificial
  siRNA
ugccucauaaacugauacctt

SEQ ID NO: 107 - DNA, 21 bp, Artificial
  siRNA
caugaacuuuauuucgccatt

SEQ ID NO: 108 - DNA, 21 bp, Artificial
  siRNA
uggcgaaauaaaguucaugtt

SEQ ID NO: 109 - DNA, 21 bp, Artificial
  siRNA
gucccuguacgagcgguuatt

SEQ ID NO: 110 - DNA, 21 bp, Artificial
  siRNA
uaaccgcucguacagggactt

SEQ ID NO: 111 - DNA, 21 bp, Artificial
  siRNA
gcgguuaagucagaggaugtt

SEQ ID NO: 112 - DNA, 21 bp, Artificial
  siRNA
cauccucugacuuaaccgctt

SEQ ID NO: 113 - DNA, 21 bp, Artificial
  siRNA
cuguacgagcgguuaaguctt

SEQ ID NO: 114 - DNA, 21 bp, Artificial
  siRNA
gacuuaaccgcucguacagtt

SEQ ID NO: 115 - DNA, 21 bp, Artificial
  siRNA
gcgaguacuucccgcuguutt

SEQ ID NO: 116 - DNA, 21 bp, Artificial
  siRNA
aacagcgggaaguacucgctt

SEQ ID NO: 117 - DNA, 21 bp, Artificial
  siRNA
gccggcaucuucuuucaugtt

SEQ ID NO: 118 - DNA, 21 bp, Artificial
  siRNA
caugaaagaagaugccggctt

SEQ ID NO: 119 - DNA, 21 bp, Artificial
  siRNA
gggucgccggcaucuucuutt

SEQ ID NO: 120 - DNA, 21 bp, Artificial
  siRNA
aagaagaugccggcgaccctt

SEQ ID NO: 121 - DNA, 21 bp, Artificial
  siRNA
ccaagauuucucuacauuutt

SEQ ID NO: 122 - DNA, 21 bp, Artificial
  siRNA
aaauguagagaaaucuuggtt

SEQ ID NO: 123 - DNA, 21 bp, Artificial
  siRNA
gauuuaugguugguagagatt

SEQ ID NO: 124 - DNA, 21 bp, Artificial
  siRNA
ucucuaccaaccauaaauctt

SEQ ID NO: 125 - DNA, 21 bp, Artificial
  siRNA
caucaugcccuucacuuaatt

SEQ ID NO: 126 - DNA, 21 bp, Artificial
  siRNA
uuaagugaagggcaugaugtt

SEQ ID NO: 127 - DNA, 21 bp, Artificial
  siRNA
guguauccucccuucuuautt

SEQ ID NO: 128 - DNA, 21 bp, Artificial
  siRNA
auaagaagggaggauacactt

SEQ ID NO: 129 - DNA, 21 bp, Artificial
  siRNA
cuggauuguuggacgaguutt

SEQ ID NO: 130 - DNA, 21 bp, Artificial
  siRNA
aacucguccaacaauccagtt

SEQ ID NO: 131 - DNA, 21 bp, Artificial
  siRNA
guguuuaccacccgcguautt

SEQ ID NO: 132 - DNA, 21 bp, Artificial
  siRNA
auacgcgggugguaaacactt

SEQ ID NO: 133 - DNA, 21 bp, Artificial
  siRNA
caucgaauuucaaccgagatt

SEQ ID NO: 134 - DNA, 21 bp, Artificial
  siRNA
ucucgguugaaauucgaugtt

SEQ ID NO: 135 - DNA, 21 bp, Artificial
  siRNA
gugauaguucaaguaagaatt

SEQ ID NO: 136 - DNA, 21 bp, Artificial
  siRNA
uucuuacuugaacuaucactt

SEQ ID NO: 137 - DNA, 21 bp, Artificial
  siRNA
ccaucaaugcacgcaagautt

SEQ ID NO: 138 - DNA, 21 bp, Artificial
  siRNA
aucuugcgugcauugauggtt

SEQ ID NO: 139 - DNA, 21 bp, Artificial
  siRNA
cagagauucagaugugguatt

SEQ ID NO: 140 - DNA, 21 bp, Artificial
  siRNA
uaccacaucugaaucucugtt

SEQ ID NO: 141 - DNA, 21 bp, Artificial
  siRNA
gccuuauuccguugguugutt

SEQ ID NO: 142 - DNA, 21 bp, Artificial
  siRNA
acaaccaacggaauaaggctt

SEQ ID NO: 143 - DNA, 21 bp, Artificial
  siRNA
gaaaugagcagguacggcatt

SEQ ID NO: 144 - DNA, 21 bp, Artificial
  siRNA
ugccguaccugcucauuuctt

SEQ ID NO: 145 - DNA, 21 bp, Artificial
  siRNA
ccuucaacuggagcauguatt

SEQ ID NO: 146 - DNA, 21 bp, Artificial
  siRNA
uacaugcuccaguugaaggtt

SEQ ID NO: 147 - DNA, 21 bp, Artificial
  siRNA
gacaucaccaugacagauutt

SEQ ID NO: 148 - DNA, 21 bp, Artificial
  siRNA
aaucugucauggugauguctt

SEQ ID NO: 149 - DNA, 21 bp, Artificial
  siRNA
cccucaucgacauugguuctt

SEQ ID NO: 150 - DNA, 21 bp, Artificial
  siRNA
gaaccaaugucgaugagggtt

SEQ ID NO: 151 - DNA, 21 bp, Artificial
  siRNA
gcaacguggccaccaucaatt

SEQ ID NO: 152 - DNA, 21 bp, Artificial
  siRNA
uugaugguggccacguugctt

SEQ ID NO: 153 - DNA, 21 bp, Artificial
  siRNA
ccagauaccugggagcguutt

SEQ ID NO: 154 - DNA, 21 bp, Artificial
  siRNA
aacgcucccagguaucuggtt

SEQ ID NO: 155 - DNA, 21 bp, Artificial
  siRNA
cgcugaaguggaugggccatt

SEQ ID NO: 156 - DNA, 21 bp, Artificial
  siRNA
uggcccauccacuucagcgtt

SEQ ID NO: 157 - DNA, 21 bp, Artificial
  siRNA
cacaagaggaccagauuaatt

SEQ ID NO: 158 - DNA, 21 bp, Artificial
  siRNA
uuaaucugguccucuugugtt

SEQ ID NO: 159 - DNA, 21 bp, Artificial
  siRNA
gcaacacgggcgagaucaatt

SEQ ID NO: 160 - DNA, 21 bp, Artificial
  siRNA
uugaucucgcccguguugctt

SEQ ID NO: 161 - DNA, 21 bp, Artificial
  siRNA
cgaauucuuauaaagcugutt

SEQ ID NO: 162 - DNA, 21 bp, Artificial
  siRNA
acagcuuuauaagaauucgtt

SEQ ID NO: 163 - DNA, 21 bp, Artificial
  siRNA
gucguuuguggauguggcatt

SEQ ID NO: 164 - DNA, 21 bp, Artificial
  siRNA
ugccacauccacaaacgactt

SEQ ID NO: 165 - DNA, 21 bp, Artificial
  siRNA
caugcaaagccaaugccgatt

SEQ ID NO: 166 - DNA, 21 bp, Artificial
  siRNA
ucggcauuggcuuugcaugtt

SEQ ID NO: 167 - DNA, 21 bp, Artificial
  siRNA
gguauucuggcaggcuucutt

SEQ ID NO: 168 - DNA, 21 bp, Artificial
  siRNA
agaagccugccagaauacctt

SEQ ID NO: 169 - DNA, 21 bp, Artificial
  siRNA
guuucuauucaguuaaagatt

SEQ ID NO: 170 - DNA, 21 bp, Artificial
  siRNA
ucuuuaacugaauagaaactt

SEQ ID NO: 171 - DNA, 21 bp, Artificial
  siRNA
gagacauuggcucuuaagatt

SEQ ID NO: 172 - DNA, 21 bp, Artificial
  siRNA
ucuuaagagccaaugucuctt

SEQ ID NO: 173 - DNA, 21 bp, Artificial
  siRNA
gaacuucgacauggugauatt

SEQ ID NO: 174 - DNA, 21 bp, Artificial
  siRNA
uaucaccaugucgaaguuctt

SEQ ID NO: 175 - DNA, 21 bp, Artificial
  siRNA
ccuauuuauucacucgacatt

SEQ ID NO: 176 - DNA, 21 bp, Artificial
  siRNA
ugucgagugaauaaauaggtt

SEQ ID NO: 177 - DNA, 21 bp, Artificial
  siRNA
gagauacgaguacgaguuutt

SEQ ID NO: 178 - DNA, 21 bp, Artificial
  siRNA
aaacucguacucguaucuctt

SEQ ID NO: 179 - DNA, 21 bp, Artificial
  siRNA
ccuuaagggacuaaauuaatt

SEQ ID NO: 180 - DNA, 21 bp, Artificial
  siRNA
uuaauuuagucccuuaaggtt

SEQ ID NO: 181 - DNA, 21 bp, Homo sapiens
aagaaccaagggcatataaag

SEQ ID NO: 182 - DNA, 21 bp, Artificial
  siRNA
gaaccaagggcauauaaagtt

SEQ ID NO: 183 - DNA, 21 bp, Artificial
  siRNA
cuuuauaugcccuugguuctt

SEQ ID NO: 184 - DNA, 21 bp, Homo sapiens
aaccaagggcatataaagaca

SEQ ID NO: 185 - DNA, 21 bp, Artificial
  siRNA
ccaagggcauauaaagacatt

SEQ ID NO: 186 - DNA, 21 bp, Artificial
  siRNA
ugucuuuauaugcccuuggtt

SEQ ID NO: 187 - DNA, 21 bp, Homo sapiens
aagggcatataaagacagatg

SEQ ID NO: 188 - DNA, 21 bp, Artificial
  siRNA
gggcauauaaagacagaugtt

SEQ ID NO: 189 - DNA, 21 bp, Artificial
  siRNA
caucugucuuuauaugccctt

SEQ ID NO: 190 - DNA, 21 bp, Homo sapiens
aaagacagatgggaggagacc

SEQ ID NO: 191 - DNA, 21 bp, Artificial
  siRNA
agacagaugggaggagacctt

SEQ ID NO: 192 - DNA, 21 bp, Artificial
  siRNA
ggucuccucccaucugucutt

SEQ ID NO: 193 - DNA, 21 bp, Homo sapiens
aagaagcatctacataggtac

SEQ ID NO: 194 - DNA, 21 bp, Artificial
  siRNA
gaagcaucuacauagguactt

SEQ ID NO: 195 - DNA, 21 bp, Artificial
  siRNA
guaccuauguagaugcuuctt

SEQ ID NO: 196 - DNA, 21 bp, Artificial
  siRNA
cgucauacuccaacuauuatt

SEQ ID NO: 197 - DNA, 21 bp, Artificial
  siRNA
uaauaguuggaguaugacgtt

SEQ ID NO: 198 - DNA, 21 bp, Artificial
  siRNA
caaaucugcugccauguuatt

SEQ ID NO: 199 - DNA, 21 bp, Artificial
  siRNA
uaacauggcagcagauuugtt

SEQ ID NO: 200 - DNA, 21 bp, Artificial
  siRNA
cgaauaugccuuggcuguutt

SEQ ID NO: 201 - DNA, 21 bp, Artificial
  siRNA
aacagccaaggcauauucgtt

SEQ ID NO: 202 - DNA, 19 bp, Artificial
  siRNA
gcuauacaauccuacccau

SEQ ID NO: 203 - DNA, 19 bp, Artificial
  siRNA
auggguaggauuguauagc

SEQ ID NO: 204 - DNA, 19 bp, Artificial
  siRNA
cugauacucuuccuuguca

SEQ ID NO: 205 - DNA, 19 bp, Artificial
  siRNA
ugacaaggaagaguaucag

SEQ ID NO: 206 - DNA, 19 bp, Artificial
  siRNA
cgaucuugguccugccaua

SEQ ID NO: 207 - DNA, 19 bp, Artificial
  siRNA
uauggcaggaccaagaucg

SEQ ID NO: 208 - DNA, 21 bp, Artificial
  siRNA
ccaguuugcuccuuggucatt

SEQ ID NO: 209 - DNA, 21 bp, Artificial
  siRNA
ugaccaaggagcaaacuggtt

SEQ ID NO: 210 - DNA, 21 bp, Artificial
  siRNA
cacugaagggccgcguggutt

SEQ ID NO: 211 - DNA, 21 bp, Artificial
  siRNA
accacgcggcccuucagugtt

SEQ ID NO: 212 - DNA, 21 bp, Artificial
  siRNA
gagacucaagcaauaauuatt

SEQ ID NO: 213 - DNA, 21 bp, Artificial
  siRNA
uaauuauugcuugagucuctt

SEQ ID NO: 214 - DNA, 21 bp, Artificial
  siRNA
cccugaaugugguguuccutt

SEQ ID NO: 215 - DNA, 21 bp, Artificial
  siRNA
aggaacaccacauucagggtt

SEQ ID NO: 216 - DNA, 21 bp, Artificial
  siRNA
gaggcuuccugaugcucuatt

SEQ ID NO: 217 - DNA, 21 bp, Artificial
  siRNA
uagagcaucaggaagccuctt

SEQ ID NO: 218 - DNA, 21 bp, Artificial
  siRNA
ggcaaugagaccaacaccutt

SEQ ID NO: 219 - DNA, 21 bp, Artificial
  siRNA
agguguuggucucauugcctt

SEQ ID NO: 220 - DNA, 21 bp, Artificial
  siRNA
caauaagaaccgagacgaatt

SEQ ID NO: 221 - DNA, 21 bp, Artificial
  siRNA
uucgucucgguucuuauugtt

SEQ ID NO: 222 - DNA, 21 bp, Artificial
  siRNA
gugaaacacugcaagcguutt

SEQ ID NO: 223 - DNA, 21 bp, Artificial
  siRNA
aacgcuugcaguguuucactt

SEQ ID NO: 224 - DNA, 21 bp, Artificial
  siRNA
gagacuuccuccaaauggutt

SEQ ID NO: 225 - DNA, 21 bp, Artificial
  siRNA
accauuuggaggaagucuctt

SEQ ID NO: 226 - DNA, 21 bp, Artificial
  siRNA
caauggaucccgagacuuutt

SEQ ID NO: 227 - DNA, 21 bp, Artificial
  siRNA
aaagucucgggauccauugtt

SEQ ID NO: 228 - DNA, 21 bp, Artificial
  siRNA
guacaauccgcauccaacutt

SEQ ID NO: 229 - DNA, 21 bp, Artificial
  siRNA
aguuggaugcggauuguactt

SEQ ID NO: 230 - DNA, 21 bp, Artificial
  siRNA
gagauauggaaucagauuatt

SEQ ID NO: 231 - DNA, 21 bp, Artificial
  siRNA
uaaucugauuccauaucuctt

SEQ ID NO: 232 - DNA, 21 bp, Artificial
  siRNA
gcauagaaugcagcaauuutt

SEQ ID NO: 233 - DNA, 21 bp, Artificial
  siRNA
aaauugcugcauucuaugctt

SEQ ID NO: 234 - DNA, 21 bp, Artificial
  siRNA
gaaagaauuugcggcaauutt

SEQ ID NO: 235 - DNA, 21 bp, Artificial
  siRNA
aauugccgcaaauucuuuctt

SEQ ID NO: 236 - DNA, 21 bp, Artificial
  siRNA
cagaguacguucgacgggatt

SEQ ID NO: 237 - DNA, 21 bp, Artificial
  siRNA
ucccgucgaacguacucugtt

SEQ ID NO: 238 - DNA, 21 bp, Artificial
  siRNA
gacgguucuuguuccaguatt

SEQ ID NO: 239 - DNA, 21 bp, Artificial
  siRNA
uacuggaacaagaaccguctt

SEQ ID NO: 240 - DNA, 21 bp, Artificial
  siRNA
ccauuaccaggauggugcatt

SEQ ID NO: 241 - DNA, 21 bp, Artificial
  siRNA
ugcaccauccugguaauggtt

SEQ ID NO: 242 - DNA, 21 bp, Artificial
  siRNA
ccguuuaucaccugaccgatt

SEQ ID NO: 243 - DNA, 21 bp, Artificial
  siRNA
ucggucaggugauaaacggtt

SEQ ID NO: 244 - DNA, 21 bp, Artificial
  siRNA
gaguaugcgaugugcuuaatt

SEQ ID NO: 245 - DNA, 21 bp, Artificial
  siRNA
uuaagcacaucgcauacuctt

SEQ ID NO: 246 - DNA, 21 bp, Artificial
  siRNA
caguauaagugcgauuguatt

SEQ ID NO: 247 - DNA, 21 bp, Artificial
  siRNA
uacaaucgcacuuauacugtt

SEQ ID NO: 248 - DNA, 21 bp, Artificial
  siRNA
guaugagugugggauuugatt

SEQ ID NO: 249 - DNA, 21 bp, Artificial
  siRNA
ucaaaucccacacucauactt

SEQ ID NO: 250 - DNA, 21 bp, Artificial
  siRNA
gacuacuucuggcauccuutt

SEQ ID NO: 251 - DNA, 21 bp, Artificial
  siRNA
aaggaugccagaaguaguctt

SEQ ID NO: 252 - DNA, 21 bp, Artificial
  siRNA
caaccuaguggagaacacatt

SEQ ID NO: 253 - DNA, 21 bp, Artificial
  siRNA
uguguucuccacuagguugtt

SEQ ID NO: 254 - DNA, 21 bp, Artificial
  siRNA
caaagcuuaccgugacaautt

SEQ ID NO: 255 - DNA, 21 bp, Artificial
  siRNA
auugucacgguaagcuuugtt

SEQ ID NO: 256 - DNA, 21 bp, Artificial
  siRNA
cugucagccucuuccgggatt

SEQ ID NO: 257 - DNA, 21 bp, Artificial
  siRNA
ucccggaagaggcugacagtt

SEQ ID NO: 258 - DNA, 21 bp, Artificial
  siRNA
cccuucagaccagcgggaatt

SEQ ID NO: 259 - DNA, 21 bp, Artificial
  siRNA
uucccgcuggucugaagggtt

SEQ ID NO: 260 - DNA, 21 bp, Artificial
  siRNA
cugaccugguguggucucatt

SEQ ID NO: 261 - DNA, 21 bp, Artificial
  siRNA
ugagaccacaccaggucagtt

SEQ ID NO: 262 - DNA, 21 bp, Artificial
  siRNA
caaguugucagggacaugatt

SEQ ID NO: 263 - DNA, 21 bp, Artificial
  siRNA
ucaugucccugacaacuugtt

SEQ ID NO: 264 - DNA, 21 bp, Artificial
  siRNA
gaaauaaccuccggagcautt

SEQ ID NO: 265 - DNA, 21 bp, Artificial
  siRNA
augcuccggagguuauuuctt

SEQ ID NO: 266 - DNA, 21 bp, Artificial
  siRNA
caguuauguuggaagauuutt

SEQ ID NO: 267 - DNA, 21 bp, Artificial
  siRNA
aaaucuuccaacauaacugtt

(19) United States
(12) Patent Application Publication
(10) Pub. No.: US 2015/0139949 A1
(43) Pub. Date: May 21, 2015

(54) ANTI-VIRAL COMBINATION THERAPY

(71) Applicants: Emre KOYUNCU, Princeton, NJ (US); Thomas E. SHENK, Princeton, NJ (US); Joshua RABINOWITZ, Princeton, NJ (US)

(72) Inventors: Emre KOYUNCU, Princeton, NJ (US); Thomas E. SHENK, Princeton, NJ (US); Joshua RABINOWITZ, Princeton, NJ (US)

(21) Appl. No.: 14/281,596

(22) Filed: May 19, 2014

Related U.S. Application Data
(63) Continuation of application No. 14/110,402, now abandoned.
(60) Provisional application No. 61/472,608, filed on Apr. 6, 2011.

(51) Int. Cl. A61K 31/7032; A61K 45/06; A61K 38/05

(52) U.S. Cl. CPC A61K 31/7032; A61K 38/05; A61K 45/06
USPC 424/85.7; 514/4.3

(57) ABSTRACT

The present invention provides methods and compounds for treating viral infections using combinations modulators of an HCV-associated component and modulators of host cell enzymes. The present invention also provides methods and compounds for treating viral infections using combinations of modulators of host cell enzymes and other agents that work, at least in part by modulating hos factors.

### CROSS-REFERENCE TO RELATED APPLICATIONS

This application claims priority to U.S. Application No. 61/472,608, filed Apr. 6, 2011, which is incorporated herein by reference in its entirety.

### FIELD OF THE INVENTION

This application relates to antiviral therapies for treatment of HCV infection.

### BACKGROUND OF THE INVENTION

There is a great unmet medical need for agents that more safely, effectively, and reliably treat viral infections, from HIV to the common cold. This includes a major need for better agents to treat human cytomegalovirus (where current agents suffer from significant toxicity and lack of efficacy), herpes simplex virus (where current agents are beneficial but provide incomplete relief), influenza A (where resistance to current agents is rampant), and hepatitis C virus (where many patients die from poor disease control). It further includes a major need for therapies that work across a spectrum of viruses, facilitating their clinical use without necessarily requiring identification of the underlying pathogen.

Persistent hepatitis C virus (HCV) infections are associated with cirrhosis and liver cancer and contribute significantly to liver-specific morbidity in human populations. In addition, infection by HCV is responsible for most transfusion-associated cases of non-A, non-B hepatitis and also accounts for a significant proportion of community-acquired hepatitis cases worldwide. Relatively few HCV-infected individuals experience acute hepatitis, but up to 85% develop persistent infection that often leads to chronic hepatitis and liver cirrhosis, eventually predisposing them to hepatocellular carcinoma. Presently, HCV vaccines are not available and no broadly effective therapies exist for persistent HCV infection.

More than 170 million people are infected HCV. The current standard of care for HCV infection, a combination of ribavirin and pegylated interferon-alpha (IFN-α), suffers from safety and adequacy issues. Common side effects of IFN-α treatment include flu like symptoms and fatigue, a decrease in the white blood count and platelet count (a blood clotting element), depression, irritability, sleep disturbances, and anxiety as well as personality changes. The most significant side effect of ribavirin is hemolytic anemia, resulting from destruction of red blood cells. Ribavarin administration also carries a risk of birth defects. Patients who are pregnant or considering becoming pregnant cannot take ribavirin, and birth control measures must be taken during treatments with ribavirin.

### SUMMARY OF THE INVENTION

The invention provides novel methods and compositions for treatment or amelioration of HCV infection and involves administration to a subject in need thereof a therapeutically effective amount of a combination therapy comprising (i) a compound that is a modulator of a host cell target or a prodrug thereof, or pharmaceutically acceptable salt or ester of said compound or prodrug and (ii) a compound that is a modulator of an HCV-associated component or a prodrug thereof, or pharmaceutically acceptable salt or ester of said compound or prodrug. Such combination therapy provides improved antiviral activity and/or reduces overall toxicity and undesirable side effects of the drugs used in the combination therapy.

Useful agents that modulate host cell targets according to the invention are inhibitors of fatty acid synthesis enzymes or cellular long and very long chain fatty acid metabolic enzymes and processes, including, but not limited to, inhibitors of ACSL1, ELOVL2, ELOVL3, ELOVL6, FAS, SLC27A3, ACC, HMG-CoA reductase, and lipid droplet formation. According to the invention, such inhibitors of cellular enzymes and processes are administered with agents that target viral enzymes.

In one embodiment the modulator of a host cell target (that is administered as part of a combination therapy with a modulator of an HCV-associated component) is a compound that is an inhibitor of acetyl-CoA carboxylase (ACC) or a prodrug thereof, or pharmaceutically acceptable salt or ester of said compound or prodrug. In one embodiment the inhibitor of ACC inhibits ACC1, ACC2, or both ACC1 and ACC2. In one embodiment the ACC inhibitor is a compound of structure XI as described herein. In one embodiment the ACC inhibitor is a compound of structure XII as described herein including, but not limited to, TOFA. In one embodiment the ACC inhibitor is a compound of structure XIII as described herein including, but not limited to, CP-610431 and CP-640186. In another embodiment the inhibitor of ACC is a compound of structure XIV as described herein including, but not limited to, Soraphen A, Soraphen B. In another embodiment the inhibitor of ACC is a compound of structure XV as described herein including, but not limited to, haloxyfop. In another embodiment the inhibitor of ACC is a compound of structure XVI as described herein including, but not limited to, sethoxydim. In another embodiment the inhibitor of ACC is a compound of structure XVII as described herein including, but not limited to,

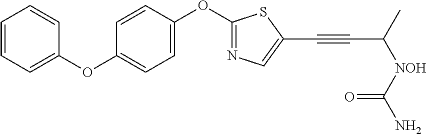

and compounds of structures XVIIa or XVIIb, as disclosed herein. In one embodiment, the compound of structure XVIIb is

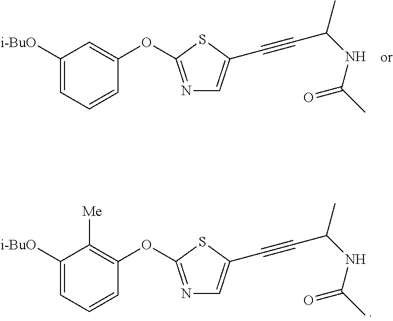

In one embodiment the modulator of a host cell target (that is administered as part of a combination therapy with a modulator of an HCV-associated component) is a compound that is an inhibitor of an acyl-CoA:cholesterol acyl-transferase (ACAT) or a prodrug thereof, or pharmaceutically acceptable salt or ester of said compound or prodrug. In one embodiment the inhibitor of ACAT inhibits ACAT1, ACAT2, or both ACAT1 and ACAT2. In one embodiment the ACAT inhibitor is pactimibe, Compound 1, Compound 21, Compound 12g, SMP-797, CL-283,546, Wu-V-23 or eflucimibe. In an other embodiment the inhibitor of ACAT is a compound of structure V as described herein including, but not limited to, avasimibe. In one embodiment the ACAT inhibitor is pactimibe, Compound 1, Compound 21, Compound 12g, SMP-797, CL-283,546, Wu-V-23 or eflucimibe.

In one embodiment the modulator of a host cell target (that is administered as part of a combination therapy with a modulator of an HCV-associated component) is a compound that is an inhibitor of a long-chain acyl-CoA synthetase (ACSL) or a prodrug thereof, or pharmaceutically acceptable salt or ester of said compound or prodrug. In one embodiment the inhibitor of ACSL is an inhibitor of one or more of ACSL1, ACSL3, ACSL4, ACSL5, and ACSL6. In one embodiment, the ACSL inhibitor is a compound of structure I as described herein. In one embodiment the ACSL inhibitor is triacsin A, triacsin B, triacsin C, or triacsin D. In one embodiment the ASCL inhibitor is a triacsin analog of structure II, structure III, structure IVa, or structure IVb as disclosed herein.

In one embodiment the modulator of a host cell target (that is administered as part of a combination therapy with a modulator of an HCV-associated component) is a compound that is an inhibitor of an elongase (ELOVL) or a prodrug thereof, or pharmaceutically acceptable salt or ester of said compound or prodrug. In one embodiment the inhibitor of ELOVL inhibits of one or more of ELOVL2, ELOVL3, and ELOVL6. In one embodiment the inhibitor of ENOVL is a compound selected from the structures VI, VIa, VIb, VIIa, VIIb, VIII, or IX as disclosed herein.

In one embodiment the modulator of a host cell target (that is administered as part of a combination therapy with a modulator of an HCV-associated component) is a compound that is an inhibitor of fatty acid synthase (FAS) or a prodrug thereof, or pharmaceutically acceptable salt or ester of said compound or prodrug. In one embodiment the inhibitor of FAS is a compound with the structure XVIII as described herein including, but not limited to, C75. In one embodiment the inhibitor of FAS is a compound with the structure XIX as described herein including, but not limited to, orlistat. In another embodiment the inhibitor of FAS is a compound of structure XX as described herein. In one embodiment the inhibitor of FAS is triclosan, epigallocatechin-3-gallate, luteolin, quercetin, kacmpfcrol or CBM-301106.

In one embodiment the modulator of a host cell target (that is administered as part of a combination therapy with a modulator of an HCV-associated component) is a compound that is an inhibitor of HMG-CoA reductase or a prodrug thereof, or pharmaceutically acceptable salt or ester of said compound or prodrug. In one embodiment, the HMG-CoA reductase inhibitor is fluvastatin, lovastatin, mevastatin, lovastatin, pravastatin, simvastatin, atorvastatin, itavastatin, or visastatin.

In one embodiment the modulator of a host cell target (that is administered as part of a combination therapy with a modulator of an HCV-associated component) is a compound that is an inhibitor of lipid droplet formation or a prodrug thereof, or pharmaceutically acceptable salt or ester of said compound or prodrug. In one embodiment, the inhibitor of lipid droplet accumulation is PF-1052, spylidone, sespendole, terpendole C, rubimaillin, Compound 7, Compound 8, Compound 9, vermisporin; beauveriolides; phenochalasins; isobisvertinol; or K97-0239.

In one embodiment the modulator of a host cell target (that is administered as part of a combination therapy with a modulator of an HCV-associated component) is a compound that is an inhibitor of serine palmitoyl transferase (SPT) or a prodrug thereof, or pharmaceutically acceptable salt or ester of said compound or prodrug. In one embodiment the inhibitor of SPT is myriocin, sphingofungin B, sphingofungin C, sphingofungin E sphingofungin F, lipoxamycin, viridiofungin A, sulfamisterin, or NA255.

The antiviral combination therapy includes the administration of (i) one or more modulators of the host cell targets described herein, and (ii) one or more modulator of an HCV-associated component. In one embodiment, the modulator of an HCV-associated component is an HCV protease inhibitor. In one embodiment, the HCV protease inhibitor is selected from boceprevir, telaprevir, ITMN-191, SCH-900518, TMC-435, BI-201335, MK-7009, VX-500, VX-813, BMS650032, VBY376, R7227, VX-985, ABT-333, ACH-1625, ACH-2684, GS-9256, GS-9451, MK-5172, and ABT-450. In one embodiments the the HCV protease inhibitor is boceprevir or telaprevir.

In one embodiment the modulator of an HCV-associated component is an HCV helicase (NS3) inhibitor selected from compounds of the structure

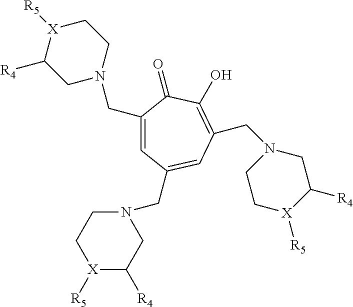

wherein X is N, R₄ is H and R₅ is CH₃; X is CH, R₄ is H and R₅ is CH₃; or X is CH, R₄ is CH₃ and R₅ is H. In another embodiment the HCV helicase (NS3) inhibitor is selected from

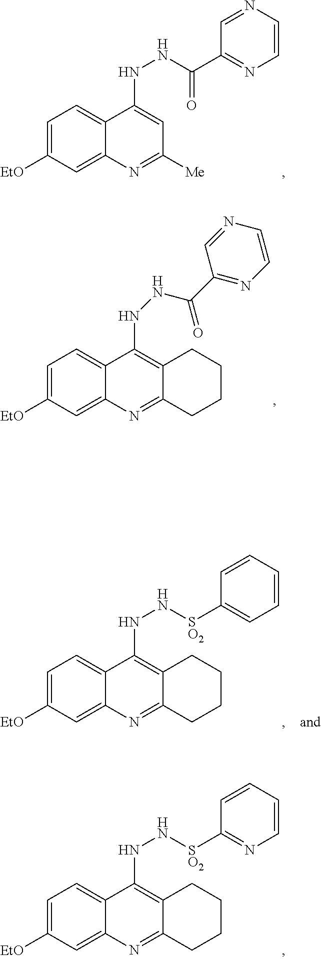 [SMILES: CCOc1ccc2c(NNC(=O)c3cnccn3)cc(C)nc2c1 ; CCOc1ccc2c(NNC(=O)c3cnccn3)c3c(nc2c1)CCCC3 ; CCOc1ccc2c(NNS(=O)(=O)c3ccccc3)c3c(nc2c1)CCCC3 ; CCOc1ccc2c(NNS(=O)(=O)c3ccccn3)c3c(nc2c1)CCCC3]

In another embodiment the HCV helicase (NS3) inhibitor is selected from

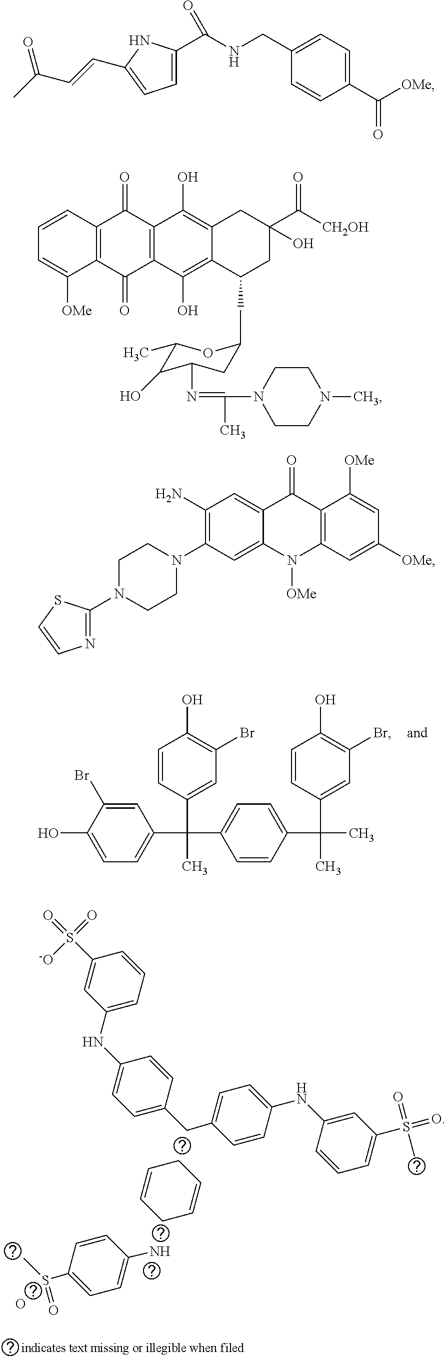

In one embodiment the modulator of an HCV-associated component is an inhibitor of HCV nonstructural protein 4B (NS4B). In one embodiment the inhibitor of NS4B is GSK-8853, clemizole, a benzimidazole RBI (B-RBI) or an indazole RBI (I-RBI).

In one embodiment the modulator of an HCV-associated component is an inhibitor HCV nonstructural protein 5A (NS5A). In one embodiment the inhibitor of NS5A is BMS-790052, A-689, A-831, EDP239, GS5885, GSK805, PPI-461 BMS-824393 or ABT-267.

In one embodiment the modulator of an HCV-associated component is an inhibitor of HCV polymerase (NS5B). In one embodiment the inhibitor of NS5B is a nucleoside analog, a nucleotide analog, or a non-nucleoside inhibitor. In one embodiment the inhibitor of NS5B is valopicitabinc, R1479, R1626, R7128, RG7128, TMC649128, IDX184, PSI-352938, INX-08189, GS6620, filibuvir, HCV-796, VCH-759, VCH-916, ANA598, VCH-222 (VX-222), BI-207127, MK-3281, ABT-072, ABT-333, GS9190, BMS791325, GSK2485852A, PSI-7851, PSI-7976, and PSI-7977.

In one embodiment the modulator of an HCV-associated component is an inhibitor of HCV viral ion channel forming protein (p7). In one embodiment the inhibitor of p7 is BIT225 or HPH116.

In one embodiment the the modulator of an HCV-associated component is an IRES inhibitor. In one embodiment the IRES inhibitor is Mifepristone, Hepazyme, ISIS14803, and siRNAs/shRNAs.

In one embodiment the the modulator of an HCV-associated component is an HCV entry inhibitor. In one embodiment the HCV entry inhibitor is HuMax HepC, JTK-652, PRO206, SP-30, or ITX5061.

In one embodiment the modulator of an HCV-associated component is a cyclosporin inhibitor. In one embodiment the cyclophilin inhibitor is Debio 025, NIM811, SCY-635, or cyclosporin-A.

In one embodiment the modulator of an HCV-associated component is modulator of microRNA-122 (miR-122). In one embodiment the modulator of microRNA-122 is SPC3649.

In one embodiment, the invention provides, in addition to the combination therapy that includes a modulator of a host cell target and a modulator of an HCV-associated component, the administration of an immunomodulator to the subject. In one embodiment the immunomodulator is one or more of Pegasys, Roferon-A, Pegintron, Intron A, Albumin IFN-α, locteron, Peginterferon-λ, omega-IFN, medusa-IFN, belerofon, infradure, Interferon alfacon-1, and Veldona.

In one embodiment, the invention provides, in addition to the combination therapy that includes a modulator of a host cell target and a modulator of an HCV-associated component, the administration to the subject one or more of ribavirin or a ribavirin analog selected from taribavirin, mizoribine, merimepodib, mycophenolate mofetil, and mycophenolate.

In one embodiment, the invention provides for treatment or amelioration of HCV infection and replication comprising a combination therapy with a modulator of a host cell target and an HCV RNAi. Such inhibitory polynucleotides include, but are not limited to, TT033, TT034, Sirna-AV34, and OBP701.

In another embodiment, the invention provides for treatment or amelioration of viral infection and replication comprising administering a combination therapy that includes a modulator of a host cell target as set forth above, and one or more agents that acts, at least partly, on another host factor. In one such embodiment, a modulator of a host cell target is administered as part of a combination therapy that includes an immunomodulator effective to reduce or inhibit HCV. Non-limiting examples of immunomodulators include inteferons (e.g., Pegasys, Pegintron, Albumin IFN-α, locteron, Peginterferon-λ, omega-IFN, medusa-IFN, belerofon, infradure, and Veldona; caspase/pan-caspase inhibitors (e.g., emricasan, nivocasan, IDN-6556, GS9450); Toll-like receptor agonists (e.g., Actilon, ANA773, IMO-2125, SD-101); cytokines and cytokine agonists and antagonists (e.g., ActoKine-2, Interleukin 29, Infliximab (cytokine TNFα blocker), IPH1101 (cytokine agonist); and other immunomodulators such as, without limitation, thymalfasin, Eltrombopag, IP1101, SCV-07, Oglufanide disodium, CYT107, ME3738, TCM-700C, EMZ702, and EGS21.

In another such embodiment, a modulator of a host cell target is administered as part of a combination therapy that includes an inhibitor of microtubule polymerization, such as, but not limited to, colchicine, GI262570, Farglitazar. Prazosin, and mitoquinone.

In another such embodiment, a modulator of a host cell target is administered as part of a combination therapy that includes a host metabolism inhibitor. Examples of host metabolism inhibitors include Hepaconda (bile acid and cholesterol secretion inhibitor), Miglustat (glucosylceramide synthase inhibitor), Celgosivir (alpha glucosidase inhibitor), Methylene blue (Monoamine oxidase inhibitor), pioglitazone and metformin (insulin regulator), Nitazoxanide (possibly PFOR inhibitor), NA255 and NA808 (Serine palmitoyltransferase inhibitor), NOV205 (Glutathione-S-transferase activator), and ADIPEG20 (arginine deiminase).

In another such embodiment, a modulator of a host cell target is administered as part of a combination therapy that includes an agent selected from laccase (herbal medicine), silibinin and silymarin (antioxidant, hepato-protective agent), PYN17 and JKB-122 (anti-inflammatory), CTS-1027 (matrix metalloproteinase inhibitor), Lenocta (protein tyrosine phosphatase inhibitor), Bavituximab and BMS936558 (programmed cell death inhibitor), HcpaCidc-I (nano-viricide), CF102 (Adenosine A3 receptor), GNS278 (inhibits viral-host protein interaction by attacking autophagy), RPIMN (Nicotinic receptor antagonist), PYN18 (possible viral maturation inhibitor), ursa and Hepaconda (bile acids, possible farnesoid X receptor), tamoxifen (anti-estrogen), Sorafenib (kinase inhibitor), KPE02001003 (unknown mechanism).

### DETAILED DESCRIPTION

The present invention is directed to combinations of modulators of host cell target enzymes with agents that act directly on the virus to treat or prevent viral infection. The present invention is also directed to combinations of modulators of host cell target enzymes with other agents that work at least partly on host factors to treat or prevent viral infection.

The invention provides novel methods and compositions for treatment or amelioration of a viral infection and involves administration to a subject in need thereof a therapeutically effective amount of combination therapy that includes (i) a compound that is a modulator of a host cell target or a prodrug thereof, or pharmaceutically acceptable salt or ester of said compound or prodrug and (ii) a compound that is a modulator of an virus-associated component or a prodrug thereof, or pharmaceutically acceptable salt or ester of said compound or prodrug. Such combination therapies provide improved antiviral activity and/or reduces overall toxicity and undesirable side effects of the drugs. In one embodiment the viral infection is by HCV.

The combination therapies of the present invention may have the advantage of producing a synergistic inhibition of viral infection or replication and, for example, allow the use of lower doses of each compound to achieve a desirable therapeutic effect. In some embodiments, the dose of one of the compounds is substantially less, e.g., 1.5, 2, 3, 5, 7, or 10-fold less, than required when used independently for the prevention and/or treatment of viral infection. In some embodiments, the dose of both agents is reduced by 1.5, 2, 3, 5, 7, or 10-fold or more. In addition to improved antiviral activity, the combination therapies of the present invention can reduce overall toxicity and undesirable side effects of the drugs by allowing the administration of lower doses of one or more of the combined compounds while providing the desired therapeutic effect.

The combination therapies of the present invention may also reduce the potential for the development of drug-resistant mutants that can occur when, for example, direct acting antiviral agents alone are used to treat viral infection.

As used herein, the term “combination,” in the context of the administration of two or more therapies to a subject, refers to the use of more than one therapy (e.g., more than one prophylactic agent and/or therapeutic agent). The use of the terms “combination” and “co-administration” do not restrict the order in which therapies are administered to a subject with a viral infection. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a viral infection.

The combination therapy of the present invention permits intermittent dosing of the individual compounds. For example, the two treatments can be administered simultaneously. Alternatively, the two treatments can be administered sequentially. In addition, the two treatments can be administered cyclically. Thus, the two or more compounds of the combination therapy may be administered concurrently for a period of time, and then one or the other administered alone.

As used herein, the term “effective amount” in the context of administering a therapy to a subject refers to the amount of a therapy which is sufficient to achieve one, two, three, four, or more of the following effects: (i) reduce or ameliorate the severity of a viral infection or a symptom associated therewith; (ii) reduce the duration of a viral infection or a symptom associated therewith; (iii) prevent the progression of a viral infection or a symptom associated therewith; (iv) cause regression of a viral infection or a symptom associated therewith; (v) prevent the development or onset of a viral infection or a symptom associated therewith; (vi) prevent the recurrence of a viral infection or a symptom associated therewith; (vii) reduce or prevent the spread of a virus from one cell to another cell, or one tissue to another tissue; (ix) prevent or reduce the spread of a virus from one subject to another subject; (x) reduce organ failure associated with a viral infection; (xi) reduce hospitalization of a subject; (xii) reduce hospitalization length; (xiii) increase the survival of a subject with a viral infection; (xiv) eliminate a virus infection; and/or (xv) enhance or improve the prophylactic or therapeutic effect(s) of another therapy.

In certain embodiments, compounds described herein may exist in several tautomeric forms. Accordingly, the chemical structures depicted herein encompass all possible tautomeric forms of the illustrated compounds. Compounds of the invention may exist in various hydrated forms.

Definitions of the more commonly recited chemical groups are set forth below. Certain variables in classes of compounds disclosed herein recite other chemical groups. Chemical groups recited herein, but not specifically defined, have their ordinary meaning as would be known by a chemist skilled in the art.

A “C_{1-X} alkyl” (or “C₁-C_{X} alkyl”) group is a saturated straight chain or branched non-cyclic hydrocarbon having from 1 to x carbon atoms. Representative —(C₁₋₈ alkyls) include -methyl, -ethyl, -n-propyl, -n-butyl, -n-pentyl, -n-hexyl, -n-heptyl and -n-octyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. A —(C_{1-X} alkyl) group can be substituted or unsubstituted.

The terms “halogen” and “halo” mean fluorine, chlorine, bromine and iodine.

An “aryl” group is an unsaturated aromatic carbocyclic group of from 6 to 14 carbon atoms having a single ring (e.g., phenyl) or multiple condensed rings (e.g., naphthyl or anthryl). Particular aryls include phenyl, biphenyl, naphthyl and the like. An aryl group can be substituted or unsubstituted.

A “heteroaryl” group is an aryl ring system having one to four heteroatoms as ring atoms in a heteroaromatic ring system, wherein the remainder of the atoms are carbon atoms. Suitable heteroatoms include oxygen, sulfur and nitrogen. In certain embodiments, the heterocyclic ring system is monocyclic or bicyclic. Non-limiting examples include aromatic groups selected from the following:

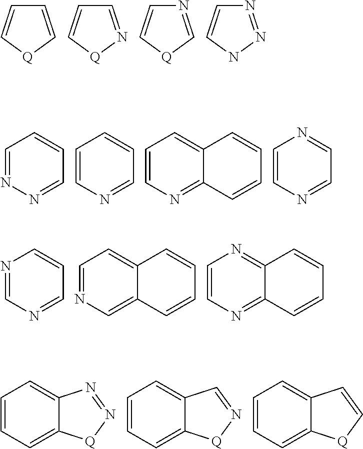

wherein Q is CH2, CH═CH, O, S or NH. Further representative examples of heteroaryl groups include, but are not limited to, benzofuranyl, benzothienyl, indolyl, benzopyrazolyl, coumarinyl, furanyl, isothiazolyl, imidazolyl, isoxazolyl, thiazolyl, triazolyl, tetrazolyl, thiophenyl, pyrimidinyl, isoquinolinyl, quinolinyl, pyridinyl, pyrrolyl, pyrazolyl, 1H-indolyl, 1H-indazolyl, benzo[d]thiazolyl and pyrazinyl. Heteroaryls can be bonded at any ring atom (i.e., at any carbon atom or heteroatom of the heteroaryl ring) A heteroaryl group can be substituted or unsubstituted. In one embodiment, the heteroaryl group is a C3-10 heteroaryl.

A “cycloalkyl” group is a saturated or unsaturated non-aromatic carbocyclic ring. Representative cycloalkyl groups include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. A cycloalkyl group can be substituted or unsubstituted. In one embodiment, the cycloalkyl group is a C3-8 cycloalkyl group.

A “heterocycloalkyl” group is a non-aromatic cycloalkyl in which one to four of the ring carbon atoms are independently replaced with a heteroatom from the group consisting of O, S and N. Representative examples of a heterocycloalkyl group include, but are not limited to, morpholinyl, pyrrolyl, pyrrolidinyl, thienyl, furanyl, thiazolyl, imidazolyl, pyrazolyl, triazolyl, piperizinyl, isothiazolyl, isoxazolyl, (1,4)-dioxane, (1,3)-dioxolane, 4,5-dihydro-1H-imidazolyl and tetrazolyl. Heterocycloalkyls can also be bonded at any ring atom (i.e., at any carbon atom or heteroatom of the Heteroaryl ring). A heterocycloalkyl group can be substituted or unsubstituted. In one embodiment, the heterocycloalkyl is a 3-7 membered heterocycloalkyl.

In one embodiment, when groups described herein are said to be “substituted,” they may be substituted with any suitable substituent or substituents. Illustrative examples of substituents include those found in the exemplary compounds and embodiments disclosed herein, as well as halogen (chloro, iodo, bromo, or fluoro); C₁₋₆ alkyl; C₂₋₆ alkenyl; C₂₋₆ alkynyl; hydroxyl; C₁₋₆ alkoxyl; amino; nitro; thiol; thioether; imine; cyano; amido; phosphonato; phosphine; carboxyl; thiocarbonyl; sulfonyl; sulfonamide; ketone; aldehyde; ester; oxygen (═O); haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); o-lower alkyl; o-aryl, aryl; aryl-lower alkyl; CO₂CH₃; CONH₂; OCH₂CONH₂; NH₂; SO₂NH₂; OCHF₂; CF₃; OCF₃.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, See for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “solvate” means a compound, or a salt thereof, that further includes a stoichiometric or non-stoichiometric amount of a solvent bound by non-covalent intermolecular forces.

As used herein and unless otherwise indicated, the term “prodrug” means a compound derivative that can hydrolyze, oxidize, or otherwise react under biological conditions (in vitro or in vivo) to provide compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound, in the context of an organic or inorganic molecule, that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

Various compounds contain one or more chiral centers, and can exist as racemic mixtures of enantiomers, mixtures of diastereomers or enantiomerically or optically pure compounds. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N.Y., 1962); and Wilen, S. H., Tables of Resolving Agents and Optical Resolutions p. 268 (E. L. Eliel, Ed., Univ. of Notre Dame Press, Notre Dame, Ind., 1972).

It should also be noted that compounds, in the context of organic and inorganic molecules, can include E and Z isomers, or a mixture thereof, and cis and trans isomers or a mixture thereof. In certain embodiments, compounds are isolated as either the E or Z isomer. In other embodiments, compounds are a mixture of the E and Z isomers.

As used herein, “small molecule” refers to a substances that has a molecular weight up to 2000 atomic mass units (Daltons). Exemplary nucleic acid-based inhibitors include siRNA and shRNA. Exemplary protein-based inhibitors include antibodies. Additional small molecule inhibitors can be found by screening of compound libraries and/or design of molecules that bind to specific pockets in the structures of these enzymes. The properties of these molecules can be optimized through derivitization, including iterative rounds of synthesis and experimental testing.

The present invention also provides for the use of the disclosed combinations in cell culture-related products in which it is desirable to have antiviral activity. In one embodiment, the combination is added to cell culture media. The compounds used in cell culture media include compounds that may otherwise be found too toxic for treatment of a subject. As used herein, the term “effective amount” in the context of a compound for use in cell culture-related products refers to an amount of a compound which is sufficient to reduce the viral titer in cell culture or prevent the replication of a virus in cell culture.

### 1. Modulators of Host Cell Target Enzymes

The invention provides cellular target enzymes for reducing virus production. Viral replication requires energy and macromolecular precursors derived from the metabolic network of the host cell. Using an integrated approach to profiling metabolic flux, the inventors discovered alterations of certain metabolite concentrations and fluxes in response to viral infection. Details of the profiling methods are described in PCT/US2008/006959, which is incorporated by reference in its entirety. Using this approach, certain enzymes in the various metabolic pathways, especially those which serve as key “switches,” have been discovered to be useful targets for intervention; i.e., as targets for redirecting the metabolic flux to disadvantage viral replication and restore normal metabolic flux profiles, thus serving as targets for antiviral therapies. Enzymes involved in initial steps in a metabolic pathway are preferred enzyme targets. In addition, enzymes that catalyze “irreversible” reactions or committed steps in metabolic pathways can be advantageously used as enzyme targets for antiviral therapy.

Accordingly, the invention provides modulators of host target enzymes useful as antiviral agents in combination with antiviral agents that act directly on viral molecules or directly act on host cell molecules that interact with viral molecules. The invention also provides modulators of host target enzymes useful as antiviral agents in combination with other agents that work at least in part by modulating host factors.

Any enzyme of a cellular metabolic pathway in which metabolite concentration and/or flux are modulated in response to viral infection is contemplated as a host cell target for antiviral intervention. In particular embodiments, host target enzymes are involved in fatty acid biosynthesis and metabolism or cellular long and very long chain fatty acid metabolism and processes, including, but not limited to, ACSL1, ELOVL2, ELOVL3, ELOVL6, FAS, SLC27A3, ACC, HMG-CoA reductase, and enzymes involved in lipid droplet formation.

The observed increase in acetyl-CoA flux (especially flux through cytosolic acetyl-CoA) and associated increase in de novo fatty acid biosynthesis, serve a number of functions for viruses, especially for enveloped viruses. For example, de novo fatty acid synthesis provides precursors for synthesis of phospholipid, and phospholipid contributes to the formation of the viral envelope, among other functions. Importantly, newly synthesized fatty acid and phospholipid may be required by the virus for purposes including control of envelope chemical composition and physical properties (e.g., phospholipid fatty acyl chain length and/or desaturation, and associated envelope fluidity). Pre-existing cellular phospholipid may be inadequate in absolute quantity, chemical composition, or physical properties to support viral growth and replication.

As such, inhibitors of any step of phospholipid biosynthesis may constitute antiviral agents. This includes steps linking initial fatty acid biosynthesis to the synthesis of fatty acyl-CoA compounds appropriate for synthesis of viral phospholipids. These steps include, but are not limited to, fatty acid elongation and desaturation. Fatty acid elongation takes the terminal product of fatty acid synthase (FAS), palmitoyl-CoA (a C16-fatty acid), and extends it further by additional two carbon units (to form, e.g., C18 and longer fatty acids). The enzyme involved is elongase. As formation of C18 and longer fatty acids is required for control of viral envelope chemical composition and physical properties, as well as for other viral functions, inhibitors of elongase may serve as inhibitors of viral growth and/or replication. Thus, in addition to compounds for treatment of viral infection by inhibition of de novo fatty acid biosynthesis enzymes (e.g., acetyl-CoA carboxylase and fatty acid synthase), the present invention also includes compounds for treatment of viral infection by inhibition of elongase and/or related enzymes of fatty acid elongation.

While inhibitors of fatty acid biosynthetic enzymes generally have utility in the treatment of viral infection, acetyl-CoA carboxylase (ACC) has specific properties that render it a useful target for the treatment of viral infection. Notably, ACC is uniquely situated to control flux through fatty acid biosynthesis. The upstream enzymes (e.g., pyruvate dehydrogenase, citrate synthase, ATP-citrate lyase, acetyl-CoA synthetase), while potential antiviral targets, generate products that are involved in multiple reaction pathways, whereas ACC generates malonyl-CoA, which is a committed substrate of the fatty acid pathway. Acetyl-CoA synthetase and ATP-citrate lyase both have the potential to generate cytosolic acetyl-CoA. Accordingly, one may, in some circumstances, partially substitute for the other. In contrast, there is no adequate alternative reaction pathway to malonyl-CoA other than carboxylation of acetyl-CoA (the ACC reaction). In this respect, targeting of ACC more completely and specifically controls fatty acid biosynthesis than targeting of upstream reactions.

As an alternative to targeting ACC, targeting FAS also enables control of fatty acid de novo biosynthesis as a whole. A key difference between targeting of ACC versus targeting of FAS, is that the substrate of ACC (acetyl-CoA) is used in numerous pathways. Accordingly, targeting ACC does not necessarily lead to marked buildup of acetyl-CoA because other pathways can consume it. In contrast, the substrate of FAS (malonyl-CoA) is used largely by FAS. Accordingly, targeting of FAS tends to lead to marked buildup of malonyl-CoA. While such buildup may in some cases have utility in the treatment of viral infection, it may in other cases contribute to side effects. Such side effects are of particular concern given (1) the important signaling and metabolism-modulating functions of malonyl-CoA and (2) lack of current FAS inhibitors with minimal in vivo side effects in mammals. The inhibition of FAS with resulting elevation in intracellular malonyl-CoA can cause cell cycle arrest with a block to cellular DNA replication and onset of apoptosis (Pizer et al., Cancer Res. 56:2745-7, 1996; Pizer et al., Cancer Res. 58:4611-5, 1998; Pizer et al., Cancer Res. 60:213-8, 2000), and it has been suggested that this toxic response can potentially account for inhibition of virus replication by FAS inhibitors (Rassmann et al., Antiviral Res. 76:150-8, 2007).

Cholesterol, like fatty acyl chain length and desaturation, plays a key role in controlling membrane/envelope physical properties like fluidity, freezing point, etc. Cholesterol percentage, like the details of phospholipid composition, can also impact the properties of membrane proteins and/or the functioning of lipid signaling. As some or all of these events play a key role in viral infection, inhibitors or other modulators of cholesterol metabolism may serve as antiviral agents. For example, inhibitors of the enzymes acetyl-CoA acetyltransferase, HMG-CoA synthase, HMG-CoA reductase, mevalonate kinase, phosphomevalonate kinase, isopentyldiphosphate isomerase, geranyl-diphosphate synthase, farnesyl-diphosphate synthase, farnesyl-diphosphate farnesyltransferase, squalene monooxigenase, lanosterol synthase, and associated demethylases, oxidases, reductase, isomerases, and desaturases of the sterol family may serve as antiviral agents.

Thus, host cell target enzymes include long and very long chain acyl-CoA synthetases and elongases as antiviral targets, including, but not limited to ACSL1, ELOVL2, ELOVL3, ELOVL6, and SLC27A3. Long-chain acyl-CoA synthetases (ACSLs) (E.C. 6.2.1.3) catalyze esterification of long-chain fatty acids, mediating the partitioning of fatty acids in mammalian cells. ACSL isoforms (ACSL1, ACSL3, ACSL4, ACSL5, and ACSL6) generate bioactive fatty acyl-CoAs from CoA, ATP, and long-chain (C₁₂-C₂₀) fatty acids. In many instances, the enzymes are tissue specific and/or substrate specific. For example, ACSLs exhibit different tissue distribution, subcellular localization, fatty acid preference, and transcriptional regulation. Similarly, seven distinct fatty acid condensing enzymes (elongases) have been identified in mouse, rat, and human, with different substrate specificities and expression patterns. ELOVL-1, ELOVL-3, and ELOVL-6 elongate saturated and monounsaturated fatty acids, whereas ELOVL-2, ELOVL-4, and ELOVL-5 elongate polyunsaturated fatty acids. ELOVL-5 also elongates some monounsaturated fatty acids, like palmitoleic acid and specifically elongates γ-linolenoyl-CoA (18:3,n-6 CoA). ELOVL-2 specifically elongates 22-carbon PUFA. Also, the elongases (ELOVL) are expressed differentially in mammalian tissues. For example, five elongases are expressed in rat and mouse liver, including ELOVL-1, -2, -3, -5, -6. In contrast, the heart expresses ELOVL-1, -5, and -6, but not ELOVL-2.

Other host cell target enzymes include, long and very long chain acyl-CoA synthetases, which can be targeted with triacsin C and its relatives, derivatives, and analogues.

Other host cell target enzymes are leukotriene C4 synthase (LTC4S), gamma-glutamyltransferase 3 (GGT3), and microsomal glutathione-S-transferase 3 (MGST3). These enzymes each contribute to the synthesis of cysteinyl leukotrienes, with LTC4S being the pivotal enzyme. In addition to siRNA, another inhibitor of cysteinyl leukotriene synthesis is caffeic acid. Synthesis of the cysteinyl leukotriene precursor leukotriene A4 can be inhibited with zileuton. According to the invention, antiviral agents also include inhibitors of leukotriene and cysteinyl leukotriene signaling, such as, but not limited to zafirlukast or montelukast.

Host cell target enzymes enzymes that are required for HCMV replication are ADP-ribosyltransferase 1 and 3 (ART1 and ART3). Inhibition of either of these enzymes led to a marked reduction in HCMV replication, ˜40-fold for ART1 and ˜10-fold for ART3. Without being bound by any particular mechanism, although ADP-ribosyltransfer is not per se a reaction of lipid metabolism, ADP ribosylation plays a key role in regulating lipid storage via targets including the protein CtBP1/BARS. Mono-ADP ribosylation of this protein results in loss of lipid droplets due to a dramatic efflux of fatty acids. Monitoring lipid droplets via microscopy with oil red O staining demonstrates that HCMV infection results initially in accumulation of lipid droplets in the infected hosts, and thereafter (by 72 hours post infection) in a dramatic depletion of lipid droplets. Accordingly, ADP-ribosylation appears to play a key role in regulating these lipid storage events during HCMV infection, and siRNA data indicates that such regulation is essential for HCMV replication. The observation that knockdown of either of these enzymes inhibited that production of infectious HCMV suggests that HCMV requires ADP-ribosyltransfer activity for efficient production of progeny virus. In addition to siRNA, another means of inhibiting ADP-ribosyltransferase is with the compound meta-iodobenzylguanidine (MIBG), and 100 μM MIBG inhibited the replication of HCMV in fibroblasts by 13-fold with no evidence of host cell toxicity.

The observations of lipid droplet accumulation and depletion during HCMV infection in an ordered temporal manner indicates that HCMV hijacks the host cell machinery involved in lipid droplet production and consumption. Thus host cell components involved in lipid droplet production and consumption provide antiviral targets. In addition to siRNA against the relevant cellular machinery, other means of inhibiting lipid droplet formation include the compounds spylidone, PF-1052 (a fungal natural product isolated from Phoma species), vermisporin, beauveriolides, phenochalasins, isobisvertinol, K97-0239, and rubimaillin. PF-1052 (10 μM) profoundly inhibited HCMV late protein synthesis (>99%) and similarly profoundly inhibits HMCV replication. In addition, triacsin C also resulted in depletion of lipid droplets, with 100 nM triacsin C causing >90% depletion of lipid droplets in HCMV infected cells and 250 nM resulting in no detectable lipid droplets by oil red O staining Normally patterns of HCMV-induced accumulation and depletion of lipid droplets were also blocked by 100 μM MIBG.

The loss of lipid droplets in HCMV infected cells is followed by the induction of lipid droplet formation in the neighboring uninfected cells. This indicates that HCMV infection results in the enhanced uptake or synthesis of lipids in the surrounding cells. Note that, HCMV spread occurs mainly from cell to cell in vivo and lipid accumulation in uninfected cells next to the infected cells can be considered as a facilitating event for the secondary infections. Triacsin C resulted in depletion of lipid droplets both in HCMV infected and surrounding uninfected cells with 100 nM triacsin C causing >90% depletion of lipid droplets and 250 nM resulting in no detectable lipid droplets by oil red O staining.

The major constituents of lipid droplets are CEs and TGs (estimated percentages in macrophages are ˜58 and ˜27 w/w respectively). Among the compounds indicated above, PF-1052 inhibits both CE and TG synthesis in a dose dependent manner, whereas, rubimaillin (also referred as mollugin) selectively inhibits CE synthesis. Rubimaillin is a naphthohydroquinone isolated from the plant Rubia Cordifoila. The inhibitory effect of rubimaillin on CE synthesis and lipid droplet formation is linked to its activity on acyl-CoA:cholesterol acyl-transferases (ACATs). It is a dual inhibitor of ACAT1 and ACAT2 enzymes (Matsuda et al., 2009, Biol. Pharm. Bull., 32, 1317-1320) and 10 μM of rubimaillin reduced HCMV replication by >80%. Thus targeting ACAT enzymes, which leads to the inhibition of lipid droplet formation, can be used in treating virus infections. The examples of dual ACAT inhibitors include the compounds pactimibe and avasimibe.

Another pair of related enzymes that are both required for HCMV replication are alanine-glyoxylate aminotransferase 2 (AGXT2) and alanine-glyoxylate aminotransferase 2-like 1 (AGXT2L1), with knockdown of AGXT2 having a particularly strong impact on viral replication. Without being bound by any particular mechanism, although alanine-glyoxylate aminotransferase is not a reaction of lipid metabolism per se, a major route of glyoxylate production in mammals is during lipid degradation. Accordingly, the antiviral effects of knockdown of AGXT2 and AGXT2L1 may arise from HCMV triggering excessive glyoxylate production which is highly reactive and toxic in biological systems from pathways including lipid degradation, and from this glyoxylate needing to be converted to glycine and pyruvate for viral replication to proceed normally. The observation that knockdown of either of these enzymes inhibits production of infectious HCMV indicates that glyoxylate degradation and/or glycine synthesis activity is required for efficient production of progeny virus and identifies alanine-glyoxylate aminotransferases as antiviral targets. In addition to siRNA, another means of inhibiting alanine-glyoxylate aminotransferase activity, which also impacts other aminotransferases, is via the compound aminooxyacetic acid (AOAA). AOAA inhibited the replication of each of three different viruses tested: HCMV, influenza A, and adenovirus.

Yet another pair of related enzymes are transaldolase 1 (TALDO1) and transketolase-like 1 (TKTL1). Although not catalyzing reactions of lipid metabolism per se, and without being bound by any particular mechanism, these enzymes both sit in the pentose phosphate pathway, which has among its major functions production of NADPH, which is used substantially for fatty acid biosynthesis. Another function of the pentose phosphate pathway which may be important for viral replication is ribose-5-phosphate synthesis. The observation that knockdown of either of these enzymes inhibited that production of infectious HCMV indicates that HCMV requires pentose phosphate pathway activity for efficient production of progeny virus. Accordingly, antiviral targets include transaldolase, transketolase, and transketolase-like enzymes.

Fatty acid elongation requires the condensation between fatty acyl-CoA and malonyl-CoA to generate β-ketoacyl-CoA which is the rate limiting step for the synthesis of long and very long chain fatty acids. This step is catalyzed by ELOVL enzymes and requires a fatty-acyl-CoA as a precursor, which is generated by ACSLs, and malonyl-CoA, which is produced by acetyl-coA carboxylase alpha (ACACA; also referred as ACCT). Therefore, in addition to ELOVLs and ACSLs, inhibition of ACACA also provides another means of inhibiting virus production. Consistently, ACACA is identified as an enzyme required for HCMV replication by the siRNA screen. In addition to siRNA, another means of inhibiting acetyl-CoA-carboxylase activity, is via the compound TOFA. TOFA inhibited the replication of each of the two different viruses: HCMV and HCV.

An enzyme which is required for HCMV replication is carbonic anhydrase 7 (CA7). Although not catalyzing the reactions of lipid metabolism per se, this enzyme catalysis the hydration of carbon dioxide to produce bicarbonate which is substantially required for the synthesis of malonyl-CoA from acetyl-coA, which is the rate limiting step of fatty acid biosynthesis. Carbonic anhydrases can be inhibited by acetazolamide, and 25 μM acetazolamide inhibited HCMV replication by ˜80% without evidence of host cell cytotoxicity.

Viral infections that direct glycolytic outflow into fatty acid biosynthesis can be treated by blockade of fatty acid synthesis. While any enzyme involved in fatty acid biosynthesis can be used as the target, the enzymes involved in the committed steps for converting glucose into fatty acid are preferred; e.g., these include, but are not limited to acetyl CoA carboxylase (ACC), its upstream regulator AMP-activated protein kinase (AMPK), or ATP citrate lyasc.

The principle pathway of production of monounsaturated fatty acids in mammals uses as major substrates palmitoyl-CoA (the product of FAS, whose production requires carboxylation of cytosolic acetyl-CoA by acetyl-CoA carboxylase [ACC]) and stearoyl-CoA (the first product of elongase). The major enzymes are Stearoyl-CoA Desaturases (SCD) 1-5 (also known generically as Fatty Acid Desaturase 1 or delta-9-desaturase). SCD isozymes 1 and 5 are expressed in primates including humans (Wang et al., Biochem. Biophys. Res. Comm. 332:735-42, 2005), and are accordingly targets for treatment of viral infection in human patients in need thereof. Other isozymes are expressed in other mammals and are accordingly targets for treatment of viral infection in species in which they are expressed. Thus, in addition to compounds for treatment of viral infection by inhibition of de novo fatty acid biosynthesis enzymes (e.g., acetyl-CoA carboxylase and fatty acid synthase), the present invention also includes compounds for treatment of viral infection by inhibition of fatty acid desaturation enzymes (e.g., SCD1, SCD5, as well as enzymes involved in formation of highly unsaturated fatty acids, e.g., delta-6-desaturase, delta-5-desaturase).

1.1 RNAi Molecules

According to the invention, RNA interference is used to reduce expression of a target enzyme in a host cell in order to reduce yield of infectious virus. siRNAs were designed to inhibit expression of a variety of enzyme targets. In certain embodiments, a compound is an RNA interference (RNAi) molecule that can decrease the expression level of a target enzyme. RNAi molecules include, but are not limited to, small-interfering RNA (siRNA), short hairpin RNA (shRNA), microRNA (miRNA), and any molecule capable of mediating sequence-specific RNAi.

RNA interference (RNAi) is a sequence specific post-transcriptional gene silencing mechanism triggered by double-stranded RNA (dsRNA) that have homologous sequences to the target mRNA. RNAi is also called post-transcriptional gene silencing or PTGS. See, e.g., Couzin, 2002, Science 298:2296-2297; McManus et al., 2002, Nat. Rev. Genet. 3, 737-747; Hannon, G. J., 2002, Nature 418, 244-251; Paddison et al., 2002, Cancer Cell 2, 17-23. dsRNA is recognized and targeted for cleavage by an RNaseIII-type enzyme termed Dicer. The Dicer enzyme “dices” the RNA into short duplexes of about 21 to 23 nucleotides, termed siRNAs or short-interfering RNAs (siRNAs), composed of 19 nucleotides of perfectly paired ribonucleotides with about two three unpaired nucleotides on the 3′ end of each strand. These short duplexes associate with a multiprotein complex termed RISC, and direct this complex to mRNA transcripts with sequence similarity to the siRNA. As a result, nucleases present in the RNA-induced silencing complex (RISC) cleave and degrade the target mRNA transcript, thereby abolishing expression of the gene product.

Numerous reports in the literature purport the specificity of siRNAs, suggesting a requirement for near-perfect identity with the siRNA sequence (Elbashir et al., 2001. EMBO J. 20:6877-6888; Tuschl et al., 1999, Genes Dev. 13:3191-3197; Hutvagner et al., Sciencexpress 297:2056-2060). One report suggests that perfect sequence complementarity is required for siRNA-targeted transcript cleavage, while partial complementarity will lead to translational repression without transcript degradation, in the manner of microRNAs (Hutvagner et al., Sciencexpress 297:2056-2060).

miRNAs are regulatory RNAs expressed from the genome, and are processed from precursor stem-loop (short hairpin) structures (approximately 80 nucleotide in length) to produce single-stranded nucleic acids (approximately 22 nucleotide in length) that bind (or hybridizes) to complementary sequences in the 3′ UTR of the target mRNA (Lee et al., 1993, Cell 75:843-854; Reinhart et al., 2000, Nature 403:901-906; Lee et al., 2001, Science 294:862-864; Lau et al., 2001, Science 294:858-862; Hutvagner et al., 2001, Science 293:834-838). miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728).

Short hairpin RNA (shRNA) is a single-stranded RNA molecule comprising at least two complementary portions hybridized or capable of hybridizing to form a double-stranded (duplex) structure sufficiently long to mediate RNAi upon processing into double-stranded RNA with overhangs, e.g., siRNAs and miRNAs. shRNA also contains at least one noncomplementary portion that forms a loop structure upon hybridization of the complementary portions to form the double-stranded structure. shRNAs serve as precursors of miRNAs and siRNAs.

Usually, sequence encoding an shRNA is cloned into a vector and the vector is introduced into a cell and transcribed by the cell's transcription machinery (Chen et al., 2003, Biochem Biophys Res Commun 311:398-404). The shRNAs can then be transcribed, for example, by RNA polymerase III (Pol III) in response to a Pol III-type promoter in the vector (Yuan et al., 2006, Mol Riot Rep 33:33-41 and Scherer et al., 2004, Mol Ther 10:597-603). The expressed shRNAs are then exported into the cytoplasm where they are processed by proteins such as Dicer into siRNAs, which then trigger RNAi (Amarzguioui et al., 2005, FEBS Letter 579:5974-5981). It has been reported that purines are required at the 5′ end of a newly initiated RNA for optimal RNA polymerase III transcription. More detailed discussion can be found in Zecherle et al., 1996, Mol. Cell. Biol. 16:5801-5810; Fruscoloni et al., 1995, Nucleic Acids Res, 23:2914-2918; and Mattaj et al., 1988, Cell, 55:435-442. The shRNAs core sequences can be expressed stably in cells, allowing long-term gene silencing in cells both in vitro and in vivo, e.g., in animals (see, McCaffrey et al., 2002, Nature 418:38-39; Xia et al., 2002, Nat. Biotech. 20:1006-1010; Lewis et al., 2002, Nat. Genetics 32:107-108; Rubinson et al., 2003, Nat. Genetics 33:401-406; and Tiscornia et al., 2003, Proc. Natl. Acad. Sci. USA 100:1844-1848).

Martinez et al. reported that RNA interference can be used to selectively target oncogenic mutations (Martinez et al., 2002, Proc. Natl. Acad. Sci. USA 99:14849-14854). In this report, an siRNA that targets the region of the R248W mutant of p53 containing the point mutation was shown to silence the expression of the mutant p53 but not the wild-type p53.

Wilda et al. reported that an siRNA targeting the M-BCR/ABL fusion mRNA can be used to deplete the M-BCR/ABL mRNA and the M-BCR/ABL oncoprotein in leukemic cells (Wilda et al., 2002, Oncogene 21:5716-5724).

U.S. Pat. No. 6,506,559 discloses a RNA interference process for inhibiting expression of a target gene in a cell. The process comprises introducing partially or fully doubled-stranded RNA having a sequence in the duplex region that is identical to a sequence in the target gene into the cell or into the extracellular environment.

U.S. Patent Application Publication No. US 2002/0086356 discloses RNA interference in a Drosophila in vitro system using RNA segments 21-23 nucleotides (nt) in length. The patent application publication teaches that when these 21-23 nt fragments are purified and added back to Drosophila extracts, they mediate sequence-specific RNA interference in the absence of long dsRNA. The patent application publication also teaches that chemically synthesized oligonucleotides of the same or similar nature can also be used to target specific mRNAs for degradation in mammalian cells.

International Patent Application Publication No. WO 2002/44321 discloses that double-stranded RNA (dsRNA) 19-23 nt in length induces sequence-specific post-transcriptional gene silencing in a Drosophila in vitro system. The PCT publication teaches that short interfering RNAs (siRNAs) generated by an RNase III-like processing reaction from long dsRNA or chemically synthesized siRNA duplexes with overhanging 3′ ends mediate efficient target RNA cleavage in the lysate, and the cleavage site is located near the center of the region spanned by the guiding siRNA.

U.S. Patent Application Publication No. US 2002/016216 discloses a method for attenuating expression of a target gene in cultured cells by introducing double stranded RNA (dsRNA) that comprises a nucleotide sequence that hybridizes under stringent conditions to a nucleotide sequence of the target gene into the cells in an amount sufficient to attenuate expression of the target gene.

International Patent Application Publication No. WO 2003/006477 discloses engineered RNA precursors that when expressed in a cell are processed by the cell to produce targeted small interfering RNAs (siRNAs) that selectively silence targeted genes (by cleaving specific mRNAs) using the cell's own RNA interference (RNAi) pathway. The PCT publication teaches that by introducing nucleic acid molecules that encode these engineered RNA precursors into cells in vivo with appropriate regulatory sequences, expression of the engineered RNA precursors can be selectively controlled both temporally and spatially, i.e., at particular times and/or in particular tissues, organs, or cells.

International Patent Application Publication No. WO 02/44321 discloses that double-stranded RNAs (dsRNAs) of 19-23 nt in length induce sequence-specific post-transcriptional gene silencing in a Drosophila in vitro system. The PCT publication teaches that siRNAs duplexes can be generated by an RNase III-like processing reaction from long dsRNAs or by chemically synthesized siRNA duplexes with overhanging 3′ ends mediating efficient target RNA cleavage in the lysate where the cleavage site is located near the center of the region spanned by the guiding siRNA. The PCT publication also provides evidence that the direction of dsRNA processing determines whether sense or antisense-identical target RNA can be cleaved by the produced siRNA complex. Systematic analyses of the effects of length, secondary structure, sugar backbone and sequence specificity of siRNAs on RNA interference have been disclosed to aid siRNA design. In addition, silencing efficacy has been shown to correlate with the GC content of the 5′ and 3′ regions of the 19 base pair target sequence. It was found that siRNAs targeting sequences with a GC rich 5′ and GC poor 3′ perform the best. More detailed discussion may be found in Elbashir et al., 2001, EMBO J. 20:6877-6888 and Aza-Blanc et al., 2003, Mol. Cell 12:627-637; each of which is hereby incorporated by reference herein in its entirety.

The invention provides specific siRNAs to target cellular components and inhibit virus replication as follows:

TABLE 1
Targets for Inhibition of Virus Replication and Inhibitory Polynucleotides

| Gene Symbol | | SEQ | | SEQ |
|---|---|---|---|---|
| (Accession No.) | siRNA (5′ to 3′ sense) | ID NO | siRNA (5′ to 3′ antisense) | ID NO |
| ACACA, transcript | GUUUGAUUGUGCCAUACUUTT | 1 | AAGUAUGGCACAAUCAAACTT | 2 |
| variant 6 | CAUGUCUGGCUUGCACCUATT | 3 | UAGGUGCAAGCCAGACAUGTT | 4 |
| (NM_000664) | GAUUGAGAAGGUUCUUAUUTT | 5 | AAUAAGAACCUUCUCAAUCTT | 6 |
| ACSL1 | GUGUGAAGAAGAAAGCUCATT | 7 | UGAGCUUUCUUCUUCACACTT | 8 |
| (NM_001995) | GAACAAGGAUGCUUUGCUUTT | 9 | AAGCAAAGCAUCCUUGUUCTT | 10 |
| | GAAAUGAAGCCAUCACGUATT | 11 | UACGUGAUGGCUUCAUUUCTT | 12 |
| AGPAT7 | CCCUCUAUGCCAACAAUGUTT | 13 | ACAUUGUUGGCAUAGAGGGTT | 14 |
| (NM_153613) | GGGUUUGGUGGACUUCCGATT | 15 | UCGGAAGUCCACCAAACCCTT | 16 |
| | CCAACAAUGUUCAGAGGGUTT | 17 | ACCCUCUGAACAUUGUUGGTT | 18 |
| AGXT2 | CAAGCUAAAGAUCAGUAUATT | 19 | UAUACUGAUCUUUAGCUUGTT | 20 |
| (NM_031900) | GUGUGAAUGGAGUUGUCCATT | 21 | UGGACAACUCCAUUCACACTT | 22 |
| | GUCUCAUGAUAGGCAUAGATT | 23 | UCUAUGCCUAUCAUGAGACTT | 24 |
| AGXT2L1 | CACCUAUGUGCUUCACUGATT | 25 | UCAGUGAAGCACAUAGGUGTT | 26 |
| (NM_031279) | GGAAUUGUCAGUUUAGAUUTT | 27 | AAUCUAAACUGACAAUUCCTT | 28 |
| | GGUUAAUAGCUCUAUUAUATT | 29 | UAUAAUAGAGCUAUUAACCTT | 30 |
| ART1 | CAACUGCGAGUACAUCAAATT | 31 | UUUGAUGUACUCGCAGUUGTT | 32 |
| (NM_004314) | CCAACCAGGUGUAUGCAGATT | 33 | UCUGCAUACACCUGGUUGGTT | 34 |
| | CAAGUCUGGGCCUUGCCAUTT | 35 | AUGGCAAGGCCCAGACUUGTT | 36 |
| ART3 | GCCAUUAUGAGUGUGCAUUTT | 37 | AAUGCACACUCAUAAUGGCTT | 38 |
| (NM_001179) | GCCAAAUGGGCAGCCCGAATT | 39 | UUCGGGCUGCCCAUUUGGCTT | 40 |
| | CUCAAAUCUUUCUCCCUAUTT | 41 | AUAGGGAGAAAGAUUUGAGTT | 42 |
| CARM1 | GUAACCUCCUGGAUCUGAATT | 43 | UUCAGAUCCAGGAGGUUACTT | 44 |
| (NM_199141) | CCAGUAACCUCCUGGAUCUTT | 45 | AGAUCCAGGAGGUUACUGGTT | 46 |
| | CCUAUGACUUGAGCAGUGUTT | 47 | ACACUGCUCAAGUCAUAGGTT | 48 |
| CDY2A | GUAAUUAAAGAAAUGGUUATT | 49 | UAACCAUUUCUUUAAUUACTT | 50 |
| (NM_004825) | GCUAUCAACUAGAUCGACATT | 51 | UGUCGAUCUAGUUGAUAGCTT | 52 |
| | GAUAAUAAAUUCAACUAUUTT | 53 | AAUAGUUGAAUUUAUUAUCTT | 54 |
| ELOVL2 | GCUACAACUUACAGUGUCATT | 55 | UGACACUGUAAGUUGUAGCTT | 56 |
| (NM_017770) | CAAAGUUUCUUUGGACCAATT | 57 | UUGGUCCAAAGAAACUUUGTT | 58 |
| | CGUUAGUCAUCCUCUUCUUTT | 59 | AAGAAGAGGAUGACUAACGTT | 60 |
| ELOVL3 | GGAGUAUUGGGCAACCUCATT | 61 | UGAGGUUGCCCAAUACUCCTT | 62 |
| (NM_152310) | GAAUGAUUAGGUUGCCUUATT | 63 | UAAGGCAACCUAAUCAUUCTT | 64 |
| | CACUUAUUCUGGUCCUUCATT | 65 | UGAAGGACCAGAAUAAGUGTT | 66 |
| ELOVL6 | GGCUUAUGCAUUUGUGCUATT | 67 | UAGCACAAAUGCAUAAGCCTT | 68 |
| (NM_024090) | CAAUGGACCUGUCAGCAAATT | 69 | UUUGCUGACAGGUCCAUUGTT | 70 |
| | CAUGUCAGUGUUGACUUUATT | 71 | UAAAGUCAACACUGACAUGTT | 72 |
| F13A1 | CUAACAAGGUGGACCACCATT | 73 | UGGUGGUCCACCUUGUUAGTT | 74 |
| (NM_000129) | CUAACCAUCCCUGAGAUCATT | 75 | UGAUCUCAGGGAUGGUUAGTT | 76 |
| | GCCUAUAGUCUCAGAGUUATT | 77 | UAACUCUGAGACUAUAGGCTT | 78 |
| GATM | GAGACAUCCUGAUAGUUGUTT | 79 | ACAACUAUCAGGAUGUCUCTT | 80 |
| (NM_001482) | CAAAUGGCUUUCCAUGAAUTT | 81 | AUUCAUGGAAAGCCAUUUGTT | 82 |
| | CAUUAAAGUUAACAUUCGUTT | 83 | ACGAAUGUUAACUUUAAUGTT | 84 |
| GGT3 | CACUCAUGACUGAGGUCAUTT | 85 | AUGACCUCAGUCAUGAGUGTT | 86 |
| (NR_003267) | CCUGUCUUGUGUGAGGUGUTT | 87 | ACACCUCACACAAGACAGGTT | 88 |
| | CCAGCAUUCACCAAUGAGUTT | 89 | ACUCAUUGGUGAAUGCUGGTT | 90 |
| GPAM | GUUAUUAGAAUGUUACGAATT | 91 | UUCGUAACAUUCUAAUAACTT | 92 |
| (NM_020918) | GAGUGUAGCAAGAGGUGUUTT | 93 | AACACCUCUUGCUACACUCTT | 94 |
| | GCAUGUUUGCCACCAAUGUTT | 95 | ACAUUGGUGGCAAACAUGCTT | 96 |
| HS6ST1 | GACGUCUUUGCAUAUGUGUTT | 97 | ACACAUAUGCAAAGACGUCTT | 98 |
| (NM_004807) | CUGUUCGAGCGGACGUUCATT | 99 | UGAACGUCCGCUCGAACAGTT | 100 |
| | CAGUACCUGUUCGAGCGGATT | 101 | UCCGCUCGAACAGGUACUGTT | 102 |
| HS6ST2 | GCCAUUUACCCAGUAUAAUTT | 103 | AUUAUACUGGGUAAAUGGCTT | 104 |
| (NM_147175) | GGUAUCAGUUUAUGAGGCATT | 105 | UGCCUCAUAAACUGAUACCTT | 106 |
| | CAUGAACUUUAUUUCGCCATT | 107 | UGGCGAAAUAAAGUUCAUGTT | 108 |
| LOC541473 | GUCCCUGUACGAGCGGUUATT | 109 | UAACCGCUCGUACAGGGACTT | 110 |
| (NR_003602) | GCGGUUAAGUCAGAGGAUGTT | 111 | CAUCCUCUGACUUAACCGCTT | 112 |
| | CUGUACGAGCGGUUAAGUCTT | 113 | GACUUAACCGCUCGUACAGTT | 114 |
| LTC4S | GCGAGUACUUCCCGCUGUUTT | 115 | AACAGCGGGAAGUACUCGCTT | 116 |
| (NM_000897) | GCCGGCAUCUUCUUUCAUGTT | 117 | CAUGAAAGAAGAUGCCGGCTT | 118 |
| | GGGUCGCCGGCAUCUUCUUTT | 119 | AAGAAGAUGCCGGCGACCCTT | 120 |
| MCCC2 | CCAAGAUUUCUCUACAUUUTT | 121 | AAAUGUAGAGAAAUCUUGGTT | 122 |
| (NM_022132) | GAUUUAUGGUUGGUAGAGATT | 123 | UCUCUACCAACCAUAAAUCTT | 124 |
| | CAUCAUGCCCUUCACUUAATT | 125 | UUAAGUGAAGGGCAUGAUGTT | 126 |
| MGST3 | GUGUAUCCUCCCUUCUUAUTT | 127 | AUAAGAAGGGAGGAUACACTT | 128 |
| (NM_004528) | CUGGAUUGUUGGACGAGUUTT | 129 | AACUCGUCCAACAAUCCAGTT | 130 |
| | GUGUUUACCACCCGCGUAUTT | 131 | AUACGCGGGUGGUAAACACTT | 132 |
| PDIA6 | CAUCGAAUUUCAACCGAGATT | 133 | UCUCGGUUGAAAUUCGAUGTT | 134 |
| (NM_005742) | GUGAUAGUUCAAGUAAGAATT | 135 | UUCUUACUUGAACUAUCACTT | 136 |
| | CCAUCAAUGCACGCAAGAUTT | 137 | AUCUUGCGUGCAUUGAUGGTT | 138 |
| PLA2G7 | CAGAGAUUCAGAUGUGGUATT | 139 | UACCACAUCUGAAUCUCUGTT | 140 |
| (NM_005084) | GCCUUAUUCCGUUGGUUGUTT | 141 | ACAACCAACGGAAUAAGGCTT | 142 |
| | GAAAUGAGCAGGUACGGCATT | 143 | UGCCGUACCUGCUCAUUUCTT | 144 |
| PNMT | CCUUCAACUGGAGCAUGUATT | 145 | UACAUGCUCCAGUUGAAGGTT | 146 |
| (NM_002686) | GACAUCACCAUGACAGAUUTT | 147 | AAUCUGUCAUGGUGAUGUCTT | 148 |
| | CCCUCAUCGACAUUGGUUCTT | 149 | GAACCAAUGUCGAUGAGGGTT | 150 |
| SLC27A3 | GCAACGUGGCCACCAUCAATT | 151 | UUGAUGGUGGCCACGUUGCTT | 152 |
| (NM_024330) | CCAGAUACCUGGGAGCGUUTT | 153 | AACGCUCCCAGGUAUCUGGTT | 154 |
| | CGCUGAAGUGGAUGGGCCATT | 155 | UGGCCCAUCCACUUCAGCGTT | 156 |
| TALDO1 | CACAAGAGGACCAGAUUAATT | 157 | UUAAUCUGGUCCUCUUGUGTT | 158 |
| (NM_006755) | GCAACACGGGCGAGAUCAATT | 159 | UUGAUCUCGCCCGUGUUGCTT | 160 |
| | CGAAUUCUUAUAAAGCUGUTT | 161 | ACAGCUUUAUAAGAAUUCGTT | 162 |
| TKTL1 | GUCGUUUGUGGAUGUGGCATT | 163 | UGCCACAUCCACAAACGACTT | 164 |
| (NM_012253) | CAUGCAAAGCCAAUGCCGATT | 165 | UCGGCAUUGGCUUUGCAUGTT | 166 |
| | GGUAUUCUGGCAGGCUUCUTT | 167 | AGAAGCCUGCCAGAAUACCTT | 168 |
| UGT3A2 | GUUUCUAUUCAGUUAAAGATT | 169 | UCUUUAACUGAAUAGAAACTT | 170 |
| (NM_174914) | GAGACAUUGGCUCUUAAGATT | 171 | UCUUAAGAGCCAAUGUCUCTT | 172 |
| | GAACUUCGACAUGGUGAUATT | 173 | UAUCACCAUGUCGAAGUUCTT | 174 |
| UST | CCUAUUUAUUCACUCGACATT | 175 | UGUCGAGUGAAUAAAUAGGTT | 176 |
| (NM_005715) | GAGAUACGAGUACGAGUUUTT | 177 | AAACUCGUACUCGUAUCUCTT | 178 |
| | CCUUAAGGGACUAAAUUAATT | 179 | UUAAUUUAGUCCCUUAAGGTT | 180 |
| SOAT1 | CGUCAUACUCCAACUAUUATT | 196 | UAAUAGUUGGAGUAUGACGTT | 197 |
| (NM_003101) | CAAAUCUGCUGCCAUGUUATT | 198 | UAACAUGGCAGCAGAUUUGTT | 199 |
| | CGAAUAUGCCUUGGCUGUUTT | 200 | AACAGCCAAGGCAUAUUCGTT | 201 |
| SOAT2 | GCUAUACAAUCCUACCCAU | 202 | AUGGGUAGGAUUGUAUAGC | 203 |
| (NM_003578) | CUGAUACUCUUCCUUGUCA | 204 | UGACAAGGAAGAGUAUCAG | 205 |
| | CGAUCUUGGUCCUGCCAUA | 206 | UAUGGCAGGACCAAGAUCG | 207 |
| CA7 | CCAGUUUGCUCCUUGGUCATT | 208 | UGACCAAGGAGCAAACUGGTT | 209 |
| (NM_005182) | CACUGAAGGGCCGCGUGGUTT | 210 | ACCACGCGGCCCUUCAGUGTT | 211 |
| | GAGACUCAAGCAAUAAUUATT | 212 | UAAUUAUUGCUUGAGUCUCTT | 213 |
| OTOP3 | CCCUGAAUGUGGUGUUCCUTT | 214 | AGGAACACCACAUUCAGGGTT | 215 |
| (NM_178233) | GAGGCUUCCUGAUGCUCUATT | 216 | UAGAGCAUCAGGAAGCCUCTT | 217 |
| | GGCAAUGAGACCAACACCUTT | 218 | AGGUGUUGGUCUCAUUGCCTT | 219 |
| TBXAS1 | CAAUAAGAACCGAGACGAATT | 220 | UUCGUCUCGGUUCUUAUUGTT | 221 |
| (NM_001061) | GUGAAACACUGCAAGCGUUTT | 222 | AACGCUUGCAGUGUUUCACTT | 223 |
| | GAGACUUCCUCCAAAUGGUTT | 224 | ACCAUUUGGAGGAAGUCUCTT | 225 |
| TYMS | CAAUGGAUCCCGAGACUUUTT | 226 | AAAGUCUCGGGAUCCAUUGTT | 227 |
| (NM_001071) | GUACAAUCCGCAUCCAACUTT | 228 | AGUUGGAUGCGGAUUGUACTT | 229 |
| | GAGAUAUGGAAUCAGAUUATT | 230 | UAAUCUGAUUCCAUAUCUCTT | 231 |
| TXNDC11 | GCAUAGAAUGCAGCAAUUUTT | 232 | AAAUUGCUGCAUUCUAUGCTT | 233 |
| (NM_015914) | GAAAGAAUUUGCGGCAAUUTT | 234 | AAUUGCCGCAAAUUCUUUCTT | 235 |
| | CAGAGUACGUUCGACGGGATT | 236 | UCCCGUCGAACGUACUCUGTT | 237 |
| PDIA5 | GACGGUUCUUGUUCCAGUATT | 238 | UACUGGAACAAGAACCGUCTT | 239 |
| (NM_006810) | CCAUUACCAGGAUGGUGCATT | 2406 | UGCACCAUCCUGGUAAUGGTT | 241 |
| | CCGUUUAUCACCUGACCGATT | 242 | UCGGUCAGGUGAUAAACGGTT | 243 |
| PTGS2 | GAGUAUGCGAUGUGCUUAATT | 244 | UUAAGCACAUCGCAUACUCTT | 245 |
| (NM_000963) | CAGUAUAAGUGCGAUUGUATT | 246 | UACAAUCGCACUUAUACUGTT | 247 |
| | GUAUGAGUGUGGGAUUUGATT | 248 | UCAAAUCCCACACUCAUACTT | 249 |
| STX8 | GACUACUUCUGGCAUCCUUTT | 250 | AAGGAUGCCAGAAGUAGUCTT | 251 |
| (NM_004853) | CAACCUAGUGGAGAACACATT | 252 | UGUGUUCUCCACUAGGUUGTT | 253 |
| | CAAAGCUUACCGUGACAAUTT | 254 | AUUGUCACGGUAAGCUUUGTT | 255 |
| OTOP2 | CUGUCAGCCUCUUCCGGGATT | 256 | UCCCGGAAGAGGCUGACAGTT | 257 |
| (NM_178160) | CCCUUCAGACCAGCGGGAATT | 258 | UUCCCGCUGGUCUGAAGGGTT | 259 |
| | CUGACCUGGUGUGGUCUCATT | 260 | UGAGACCACACCAGGUCAGTT | 261 |
| STX6 | CAAGUUGUCAGGGACAUGATT | 262 | UCAUGUCCCUGACAACUUGTT | 263 |
| (NM_005819) | GAAAUAACCUCCGGAGCAUTT | 264 | AUGCUCCGGAGGUUAUUUCTT | 265 |
| | CAGUUAUGUUGGAAGAUUUTT | 266 | AAAUCUUCCAACAUAACUGTT | 267 |

In addition, siRNA design algorithms are disclosed in PCT publications WO 2005/018534 A2 and WO 2005/042708 A2; each of which is hereby incorporated by reference herein in its entirety. Specifically, International Patent Application Publication No. WO 2005/018534 A2 discloses methods and compositions for gene silencing using siRNA having partial sequence homology to its target gene. The application provides methods for identifying common and/or differential responses to different siRNAs targeting a gene. The application also provides methods for evaluating the relative activity of the two strands of an siRNA. The application further provides methods of using siRNAs as therapeutics for treatment of diseases. International Patent Application Publication No. WO 2005/042708 A2 provides a method for identifying siRNA target motifs in a transcript using a position-specific score matrix approach. It also provides a method for identifying off-target genes of an siRNA using a position-specific score matrix approach. The application further provides a method for designing siRNAs with improved silencing efficacy and specificity as well as a library of exemplary siRNAs.

Design software can be use to identify potential sequences within the target enzyme mRNA that can be targeted with siRNAs in the methods described herein. See, for example, http://www.ambion.com/techlib/misc/siRNA_finder.html (“Ambion siRNA Target Finder Software”). For example, the nucleotide sequence of ACSL1, which is known in the art (GenBank Accession No. NM_{—}001995) is entered into the Ambion siRNA Target Finder Software (http://www.ambion.com/techlib/misc/siRNA_finder.html), and the software identifies potential ACSL1 target sequences and corresponding siRNA sequences that can be used in assays to inhibit human ACSL1 activity by downregulation of ACSL1 expression. Using this method, non-limiting examples of ACSL1 target sequence (5′ to 3′) and corresponding sense and antisense strand siRNA sequences (5′ to 3′) for inhibiting ACSL1 are identified and presented below:

| | ACSL1 Target Sequence | Sense Strand siRNA | Antisense Strand siRNA |
|---|---|---|---|
| 1. | AAGAACCAAGGGCATATAAAG | GAACCAAGGGCAUAUAAAGtt | CUUUAUAUGCCCUUGGUUCtt |
| | (SEQ ID NO: 181) | (SEQ ID NO: 182) | (SEQ ID NO: 183) |
| 2. | AACCAAGGGCATATAAAGACA | CCAAGGGCAUAUAAAGACAtt | UGUCUUUAUAUGCCCUUGGtt |
| | (SEQ ID NO: 184) | (SEQ ID NO: 185) | (SEQ ID NO: 186) |
| 3. | AAGGGCATATAAAGACAGATG | GGGCAUAUAAAGACAGAUGtt | CAUCUGUCUUUAUAUGCCCtt |
| | (SEQ ID NO: 187) | (SEQ ID NO: 188) | (SEQ ID NO: 189) |
| 4. | AAAGACAGATGGGAGGAGACC | AGACAGAUGGGAGGAGACCtt | GGUCUCCUCCCAUCUGUCUtt |
| | (SEQ ID NO: 190) | (SEQ ID NO: 191) | (SEQ ID NO: 192) |
| 5. | AAGAAGCATCTACATAGGTAC | GAAGCAUCUACAUAGGUACtt | GUACCUAUGUAGAUGCUUCtt |
| | (SEQ ID NO: 193) | (SEQ ID NO: 194) | (SEQ ID NO: 195) |

The same method can be applied to identify target sequences of any enzyme and the corresponding siRNA sequences (sense and antisense strands) to obtain RNAi molecules.

In certain embodiments, a compound is an siRNA effective to inhibit expression of a target enzyme, e.g., ACSL1 or ART1, wherein the siRNA comprises a first strand comprising a sense sequence of the target enzyme mRNA and a second strand comprising a complement of the sense sequence of the target enzyme, and wherein the first and second strands are about 21 to 23 nucleotides in length. In some embodiments, the siRNA comprises first and second strands comprise sense and complement sequences, respectively, of the target enzyme mRNA that is about 17, 18, 19, or 20 nucleotides in length.

The RNAi molecule (e.g., siRNA, shRNA, miRNA) can be both partially or completely double-stranded, and can encompass fragments of at least 18, at least 19, at least 20, at least 21, at least 22, at least 23, at least 24, at least 25, at least 30, at least 35, at least 40, at least 45, and at least 50 or more nucleotides per strand. The RNAi molecule (e.g., siRNA, shRNA, miRNA) can also comprise 3′ overhangs of at least 1, at least 2, at least 3, or at least 4 nucleotides. The RNAi molecule (e.g., siRNA, shRNA, miRNA) can be of any length desired by the user as long as the ability to inhibit target gene expression is preserved.

RNAi molecules can be obtained using any of a number of techniques known to those of ordinary skill in the art. Generally, production of RNAi molecules can be carried out by chemical synthetic methods or by recombinant nucleic acid techniques. Methods of preparing a dsRNA are described, for example, in Ausubel et al., Current Protocols in Molecular Biology (Supplement 56), John Wiley & Sons, New York (2001); Sambrook et al., Molecular Cloning—A Laboratory Manual, 3.sup.rd ed., Cold Spring Harbor Press, Cold Spring Harbor (2001); and can be employed in the methods described herein. For example, RNA can be transcribed from PCR products, followed by gel purification. Standard procedures known in the art for in vitro transcription of RNA from PCR templates. For example, dsRNA can be synthesized using a PCR template and the Ambion T7 MEGASCRIPT, or other similar, kit (Austin, Tex.); the RNA can be subsequently precipitated with LiCl and resuspended in a buffer solution.

To assay for RNAi activity in cells, any of a number of techniques known to those of ordinary skill in the art can be employed. For example, the RNAi molecules are introduced into cells, and the expression level of the target enzyme can be assayed using assays known in the art, e.g., ELISA and immunoblotting. Also, the mRNA transcript level of the target enzyme can be assayed using methods known in the art, e.g., Northern blot assays and quantitative real-time PCR. Further the activity of the target enzyme can be assayed using methods known in the art and/or described herein in section 5.3. In a specific embodiment, the RNAi molecule reduces the protein expression level of the target enzyme by at least about 10%, 20%, 30%, 40%, 50%, 60%, 70%, 80%, 90%, or 95%. In one embodiment, the RNAi molecule reduces the mRNA transcript level of the target enzyme by at least about 10%, 20%, 30%, 40%, 50%, 60%, 70%, 80%, 90%, or 95%. In a particular embodiment, the RNAi molecule reduces the enzymatic activity of the target enzyme by at least about 10%, 20%, 30%, 40%, 50%, 60%, 70%, 80%, 90%, or 95%.

1.2 Small Molecules

1.2.1 Triacsin Compounds

In one embodiment, the present invention provides a method of treating or preventing a viral infection in a subject, comprising administering to a subject in need therefore a therapeutically effective amount of triacsin C or a relative, analogue, or derivative thereof.

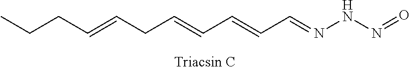

Triacsin C exists in two tautomeric forms as follows:

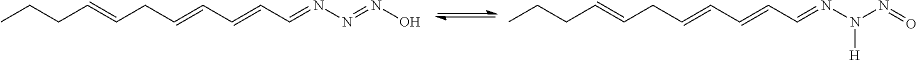

Triacsin C is a fungal antimetabolite that inhibits long chain acyl-CoA synthetases (ACSLs), arachidonoyl-CoA synthetase, and triglyceride and cholesterol ester biosynthesis. It is a member of a family of related compounds (Triacsins A-D) isolated from the culture filtrate of Streptomyces sp. SK-1894 (Omura et al., J Antibiot 39, 1211-8, 1986; Tomoda et al., Biochim Biophys Acta, 921, 595-8, 1987), all of which consist of 11-carbon alkenyl chains with a common triazenol moiety at their termini. Structures of of triacsins A, B, and D are as follows:

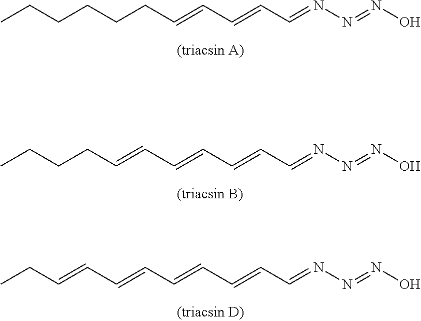

According to the invention, triacsin C or a related compound or analog or prodrug thereof, is used for treating or preventing infection by a wide range of viruses, such as, but not limited to, DNA viruses (double stranded and single stranded), double-stranded RNA viruses, single-stranded RNA viruses (negative-sense and positive-sense), single-stranded RNA retroviruses, and double stranded viruses with RNA intermediates. For example, nanomolar concentrations of triacsin C inhibit the replication of HCMV (a Herpesvirus; comprising a double stranded DNA genome), herpes simplex virus-1 (HSV-1), influenza A (an Orthomyxovirus; a negative-sense single-stranded RNA virus) and hepatitis C virus (HCV). Further, triacsin C exhibits broad spectrum anti-viral activity against enveloped viruses. Accordingly, in one embodiment of the invention, Triacsin C is used for treating or preventing infection by an enveloped virus. Also, triacsin C is active against non-enveloped viruses whose replication occurs on host cell membrane structures and against viruses that induce increases in host cell membrane.

Triacsin C inhibits ACSLs and also inhibits arachidonoyl-CoA synthase. Triacsin C inhibits triacylglycerol (TG) and cholesterol ester (CE) synthesis with an IC₅₀ of 100 nM and 190 nM, respectively. Triacsin C inhibits ACSLs in rat liver cell sonicates with an ICso of about 8.7 μM and also inhibits arachidonoyl-CoA sythethase.

Nanomolar concentrations of triacsin C inhibited by >10-fold the replication of 3 of 4 viruses tested: HCMV, herpes simplex virus-1 (HSV-1), and influenza A (but not adenovirus). HCMV, HSV-1, and influenza A (but not adenovirus) have a lipid envelope.

Triacsin C relatives that the present invention include without limitation triacsins A, C, D and WS-1228 A and B (Omura et al., J. Antibiot 39, 1211-8, 1986). Triacsin C analogues of the present invention include without limitation 3 to 25 carbon unbranched (linear) carbon chains with the triazenol moiety of triacsin C at their termini and with any combination of cis or trans double bonds in the carbon chain. In certain embodiments of the invention, the carbon chain is no shorter than 4, 5, 6, 7, 8, 9, 10, or 11 carbon atoms. In certain embodiments, the carbon chain is no longer than 24, 23, 22, 21, 20, 19, 18, 17, 16, 15, 14, 13, 12, or 11 atoms. In certain embodiments, the carbon chain contains exactly 0, 1, 2, 3, or 4 cis double bonds. In certain embodiments, the carbon chain contains exactly 0, 1, 2, 3, 4, 5, or 6 trans double bonds. In certain embodiments, as in triacsin C, there is a trans double bond at the 2^{nd} carbon-carbon bond in the chain (numbering where the carbon-nitrogen bound is bond 0). In other embodiments, there are one or more trans double bonds at bonds 3, 4, 5, 6, 7, 8, 9, 10, 11, or 12 in the chain. In certain embodiments, as in triacsin C, there is a cis-double bond at the 7^{th} carbon-carbon bond in the chain. In other embodiments, there are one or more cis double bonds at bonds 3, 4, 5, 6, 7, 8, 9, 10, 11, or 12 in the chain. Triacsin C derivatives of the present invention include without limitation triacsin or its analogues with insertion of heteroatoms or methyl or ethyl groups in place of hydrogen atoms at any point in the carbon chain. They further include variants where a portion of the linear chain of carbon-carbon bonds is replaced by one or more 3, 4, 5, or 6 membered rings, comprised of saturated or unsaturated carbon atoms or heteroatoms. A synthetic route to this class of compounds is described in U.S. Pat. No. 4,297,096 to Yoshida et al.

In certain embodiments, the triacin analogs of the invention include compounds of formula I:

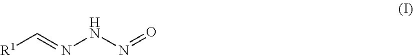

wherein R¹ is a carbon chain having from 3 to 23 atoms (including optional heteroatoms) in the chain, wherein the chain comprises
0-10 double bonds within the chain; and
0-4 heteroatoms within the chain;
and wherein 0-8 of the carbon atoms of R¹ are optionally substituted.

If one or more optional heteroatoms occur within the R¹ chain, in preferred embodiments each heteroatom is independently selected from O, S, and NR², wherein R² is selected from H, C₁₋₆ alkyl, and C₃₋₆ cycloalkyl.

When the carbon atoms of R¹ are substituted, it is preferred that from 0-8 hydrogen atoms along the chain may be replaced by a substituent selected from halo, OR², SR², lower alkyl, and cycloalkyl, wherein R² is H, C₁₋₆ alkyl, and C₃₋₆ cycloalkyl. In certain preferred embodiments, R¹ is unsubstituted (i.e., R¹ is unbranched, and none of the hydrogens have been replaced by a substituent).

In preferred embodiments for compounds of the formula I, R¹ has a chain length of 8 to 12 atoms. More preferably, R¹ has a total chain length of R¹ has a chain length of 9 to 11 atoms. Most preferably R¹ has a chain length of 10 atoms. In other preferred embodiments, R¹ has 2 to 4 double bonds.

In certain embodiments, the triacin anolog is selected from

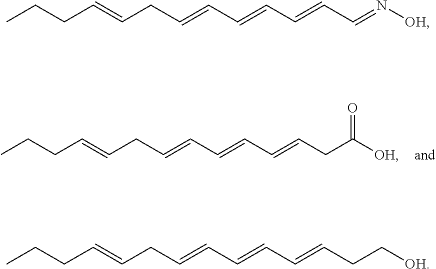 [SMILES: CCC/C=C/C/C=C/C=C/C=C/C=N/O ; CCC/C=C/C/C=C/C=C/C=C/CC(=O)O ; "and" ; CCC/C=C/C/C=C/C=C/C=C/CCO]

In certain embodiments, the triacin analogs of the invention include compounds of formula II:

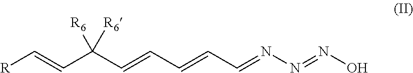

wherein R is selected from C₁₋₆ alkyl; and
wherein R₆ and R_{6′} are independently selected from H, C₁₋₃ alkyl; or R₆ and R_{6′} taken together form a cycloalkyl group of formula —(CH₂)ₙ wherein n is 2-6. In certain embodiments R may be selected from Me, Et, n-butyl, i-propyl, n-pentyl to n-hexyl. In certain embodiments, R₆ and R_{6′} are independently selected from Me and F; or R₆ and R_{6′} taken together form a cycloalkyl group of formula —(CH₂). wherein n is 2, 3, 4, and 6.

For example, in certain embodiments the triacin analog of formula II is one of the following compounds:

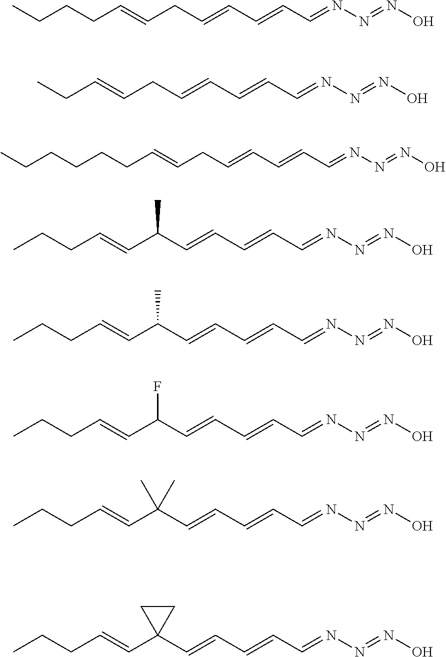

In certain embodiments, the triacin analogs of the invention include compounds of formula III:

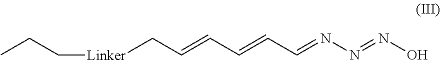

Wherein the Linker is selected from Z or E-olefin, alkyne, optionally substituted phenyl ring or optionally substituted heteroaryl ring (such as pyridine).

For example, compounds of formula III include:

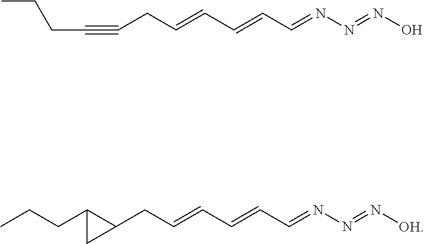 [SMILES: CCCC#CC/C=C/C=C/C=N/N=N/O ; CCCC1CC1C/C=C/C=C/C=N/N=N/O]

In another embodiment triacin analogs of the invention include compounds of formula IVa and IVb:

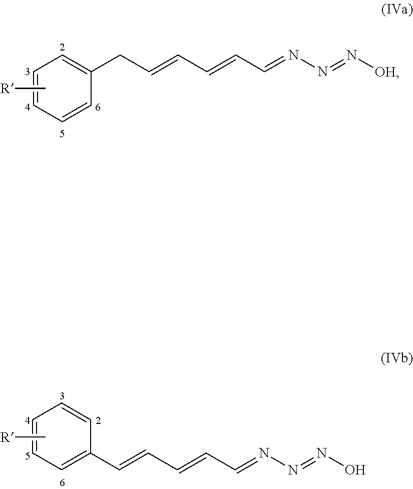

Wherein R′ is C₁₋₄ alkyl. In certain embodiments R′ is Me, Et, nPr, iPr, nBu. In certain embodiments one of the phenyl carbons at positions 2-6 may be replaced by N.

For example, in certain embodiments compounds of formula IVa include:

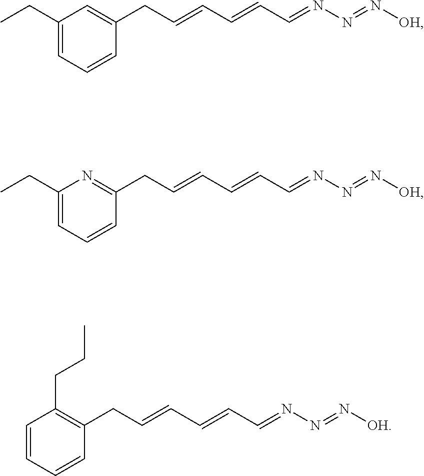 [SMILES: CCc1cccc(C/C=C/C=C/C=N/N=N/O)c1 ; CCc1cccc(C/C=C/C=C/C=N/N=N/O)n1 ; CCCc1ccccc1C/C=C/C=C/C=N/N=N/O]

In certain embodiments compounds of formula IVb include:

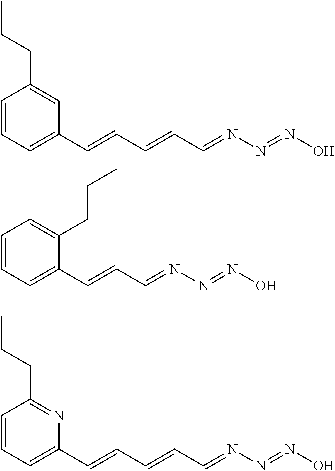

In one embodiment triacsin C analogs are designed from corresponding lipophillic tail groups, spacer groups, and polar groups

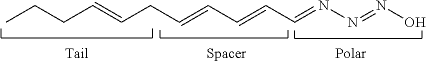

wherein the lipophilic tail group is selected from the tail group of traicin A-D and

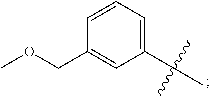 [SMILES: *c1cccc(COC)c1]

wherein the spacer group is selected from the spacer group of traicin A-D and

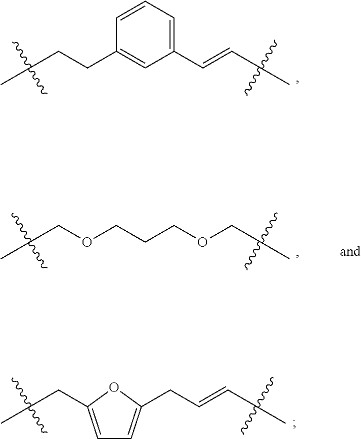 [SMILES: */C=C/c1cccc(CC*)c1 ; *COCCCOC* ; */C=C/Cc1ccc(C*)o1]

and
wherein the polar group is selected from the polar group of traicin A-D and

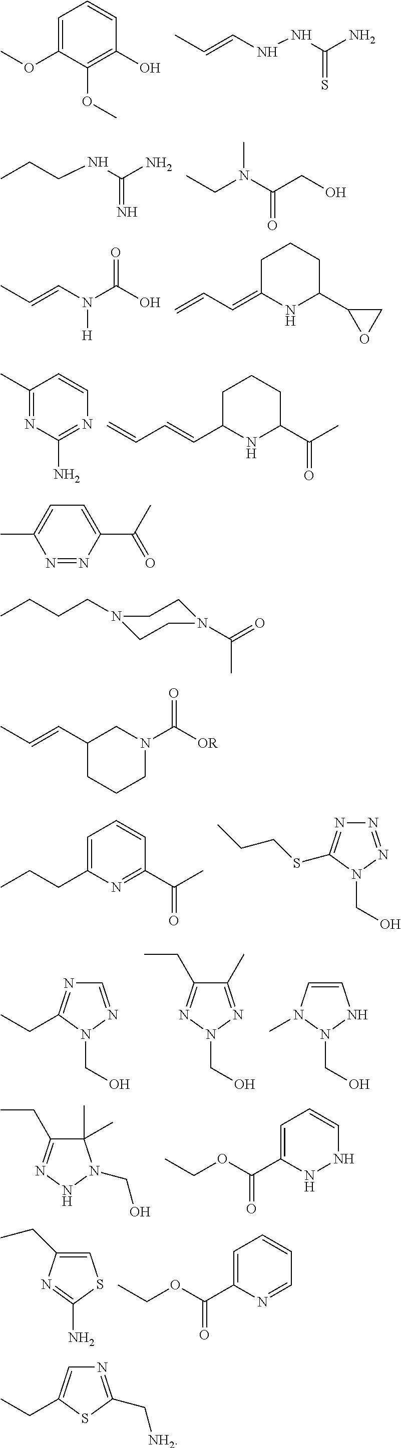

In one embodiment, the triacin C analog composed of the tail, spacer and polar group is

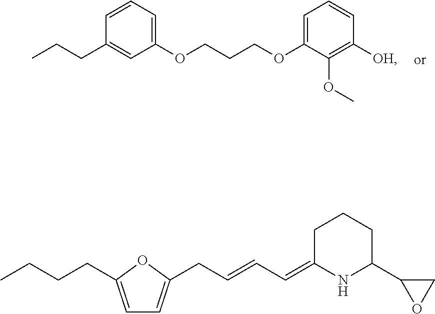 [SMILES: CCCc1cccc(OCCCOc2cccc(O)c2OC)c1 ; CCCCc1ccc(C/C=C/C=C2\CCCC(C3CO3)N2)o1]

1.2.2 Inhibitors of Lipid Drop Formation

Inhibitors of lipid drop formation include, but are not limited to the following compounds:

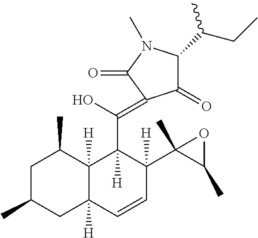

### PF-1052 (CAS: 147317-15-5)

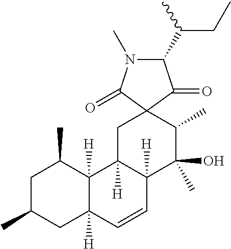

Spylidone (Liquid Droplet inhibition IC₅₀ 42 uM) (Tomoda et al., 2007, Pharmacol. Ther. 115:375-89);

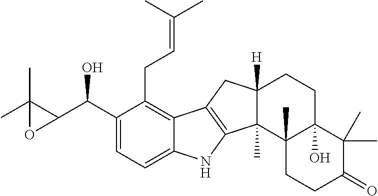

Sespendole (Liquid Droplet inhibition IC50 4 uM) (Tomoda et al., 2007, Pharmacol. Ther. 115:375-89);

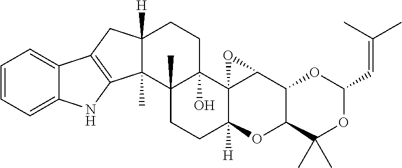

Terpendolc C (Liquid Droplet inhibition IC₅₀ 2.5 μM)(Tomoda et al., 2007, Pharmacol. Ther. 115:375-89);

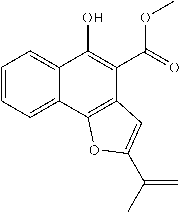 [SMILES: C=C(C)c1cc2c(C(=O)OC)c(O)c3ccccc3c2o1]

Compound 7 (Sastry et al., 2010, J. Org. Chem. 75:2274-80);

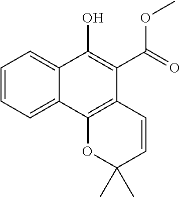 [SMILES: COC(=O)c1c2c(c3ccccc3c1O)OC(C)(C)C=C2]

### Rubimaillin;

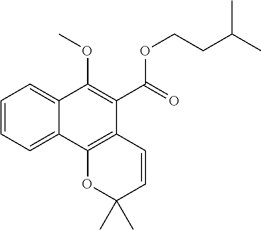 [SMILES: COc1c(C(=O)OCCC(C)C)c2c(c3ccccc13)OC(C)(C)C=C2]

Compound 8 (Ho, L. K. et al., 1996, J. Nat. Prod. 59:330-3); an

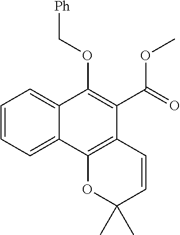 [SMILES: COC(=O)c1c2c(c3ccccc3c1OCc1ccccc1)OC(C)(C)C=C2]

Compound 9 (Ho, L. K. et al., 1996, J. Nat. Prod. 59:330-3).

Analogs of PF-1052 and Spylidone useful in the present invention include

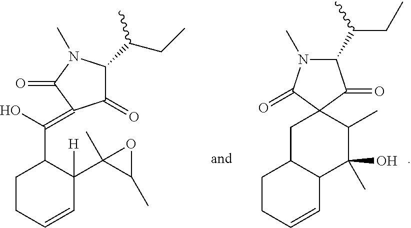

Additional inhibitors of lipid droplet formation include Vermisporin; Beauveriolides; Phenochalasins; Tsobisvertinol; and K97-0239.

1.2.3 ACAT Inhibitors

In certain embodiments, the ACAT inhibitors of the invention include compounds of formula V as follows:

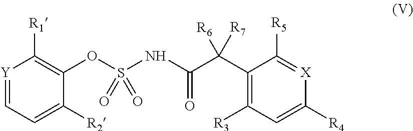

wherein

X and Y are independently selected from N and CH;

R_{1′} and R_{2′} are independently selected from H, C₁₋₆ alkyl which may be optionally substituted with F, OCH₃ and OH, and C₁₋₆ cycloalkyl;

R₆ and R₇ are independently selected from H, and C₁₋₃ alkyl, or R₆ and R₇ taken together may form a C₃₋₆ cycloalkyl;

R₃, R₄ and R₅ are independently selected from H, C₁₋₆ alkyl which may be optionally substituted with F, OCH₃ and OH, and C₁₋₆ cycloalkyl;

additionally or alternatively, one of R₆ or R₇ may be taken together with R₅ to form a C₅₋₁₁ cycloalkyl ring.

In certain embodiments, R_{1′} and/or R_{2′} are independently selected from branched C₃₋₅ alkyl and particularly isopropyl.

In certain embodiments, R₃, R₄ and/or R₅ are independently selected from branched C₃₋₅ alkyl and particularly isopropyl.

In certain embodiments, R₆ and R₇ are both H.

In certain embodiments, the ACAT inhibitors of the invention include compounds of formula Va

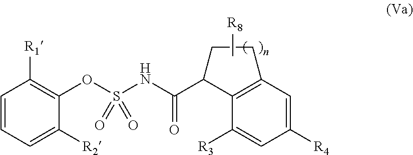

wherein

R_{1′} and R_{2′} are independently selected from H, C₁₋₆ alkyl which may be optionally substituted with F, OCH₃ and OH, and C₁₋₆ cycloalkyl;

R₃ and R₄ are independently selected from H, C₁₋₆ alkyl which may be optionally substituted with F, OCH₃ and OH, and C₁₋₆ cycloalkyl;

n is selected from 1 to 7; and

R₈ is selected from H and C₁₋₃ alkyl.

In certain embodiments, R_{1′} and/or R_{2′} are independently selected from branched C₃₋₅ alkyl and particularly isopropyl.

In certain embodiments, R₃ and/or R₄ are independently selected from branched C₃₋₅ alkyl and particularly isopropyl.

In certain embodiments, R₈ is methyl.

In one embodiment the compound of formula V is

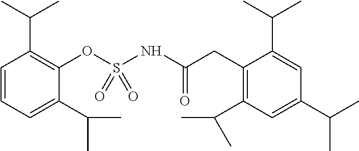 [SMILES: CC(C)c1cc(C(C)C)c(CC(=O)NS(=O)(=O)Oc2c(C(C)C)cccc2C(C)C)c(C(C)C)c1]

### Avasimibe (ACAT IC₅₀ 479 nM).

Additional ACAT inhibitors of the invention include, but are not limited to the following compounds:

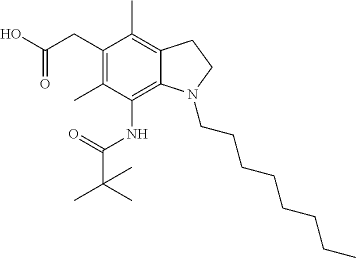 [SMILES: CCCCCCCCN1CCc2c(C)c(CC(=O)O)c(C)c(NC(=O)C(C)(C)C)c21]

Pactimibe (Liver ACAT IC₅₀ 312 nM) (Ohta et al., 2010, Chem. Pharm. Bull. 58:1066-76);

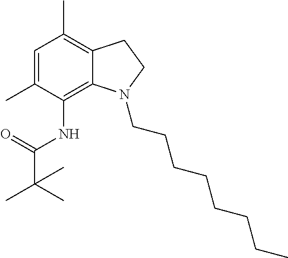 [SMILES: CCCCCCCCN1CCc2c(C)cc(C)c(NC(=O)C(C)(C)C)c21]

Compound 1 (Liver ACAT IC₅₀ 120 nM) (Takahashi et al., 2008, J. Med. Chem. 51:4823-33);

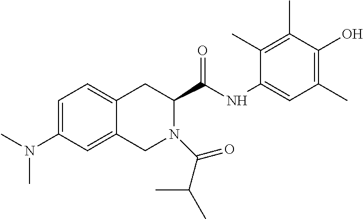

Compound 21 (Liver ACAT IC₅₀ 113 nM) (Ohta et al., 2010, Chem. Pharm. Bull. 58:1066-76);

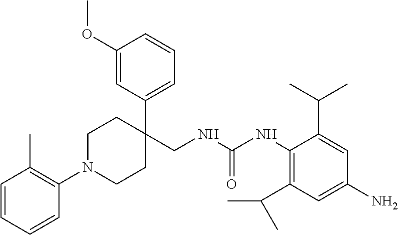 [SMILES: COc1cccc(C2(CNC(=O)Nc3c(C(C)C)cc(N)cc3C(C)C)CCN(c3ccccc3C)CC2)c1]

Compound 12g (ACAT IC₅₀ 68 nM) (Asano et al., 2009, Bioorg. Med. Chem. Lett. 19:1062-5);

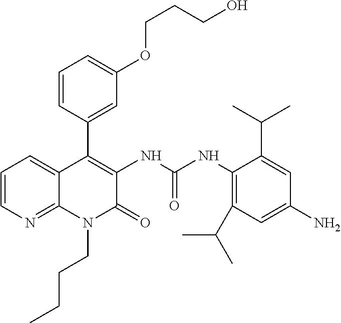 [SMILES: CCCCn1c(=O)c(NC(=O)Nc2c(C(C)C)cc(N)cc2C(C)C)c(-c2cccc(OCCCO)c2)c2cccnc21]

SMP-797 (ACAT IC₅₀ 31 nM) (Asano et al., 2009, Bioorg. Med. Chem. Lett. 19:1062-5);

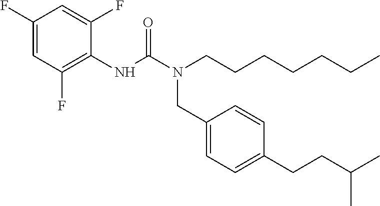 [SMILES: CCCCCCCN(Cc1ccc(CCC(C)C)cc1)C(=O)Nc1c(F)cc(F)cc1F]

CL-283,546 (Liquid Droplet inhibition IC₅₀ 35 nM) (Tomoda et al., 2007, Pharmacol. Ther. 115:375-89);

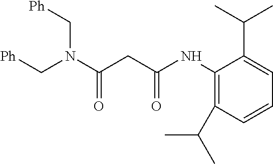 [SMILES: CC(C)c1cccc(C(C)C)c1NC(=O)CC(=O)N(Cc1ccccc1)Cc1ccccc1]

Wu-V-23 (Tomoda et al., 2007, Pharmacol. Ther. 115:375-89); and

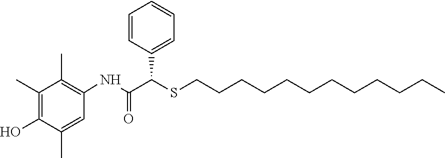 [SMILES: CCCCCCCCCCCCS[C@H](C(=O)Nc1cc(C)c(O)c(C)c1C)c1ccccc1]

### Eflucimibe.

1.2.4 Elongase Inhibitors

One example of an elongase inhibitor is a compound of formula VI:

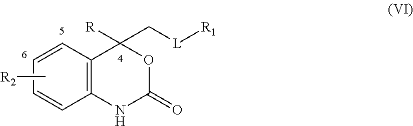

wherein L is selected from carbamate, urea, or amide including, for example

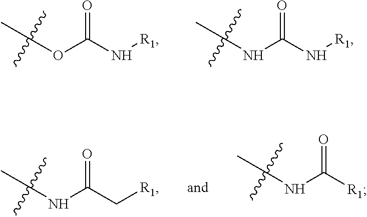

and wherein R is selected from halo; CF3; cyclopropyl; optionally substituted C₁₋₅ alkyl, wherein the C₁₋₅ alkyl may be substituted with halo, oxo, —OH, —CN, —NH₂, CO₂H, and C₁₋₃ alkoxy;
wherein R₁ is selected from substituted phenyl where the substituents are selected from F, CF₃, Me, OMe, or isopropyl;
wherein R₂ is Cl, Ph, 1-(2-pyridone), 4-isoxazol, 3-pyrazol, 4-pyrazol, 1-pyrazol, 5-(1,2,4-triazol), 1-(1,2,4-triaol), 2-imidazolo, 1-(2-pyrrolidone), 3-(1,3-oxazolidin-2-one).
The chiral center at C4 can be racemic, (S), (R), or any ratio of enantiomers. In one embodiment, L is an amide. In certain embodiments, R is selected from Cl, CF₃, methyl, ethyl, isopropyl and, cyclopropyl. In certain embodiments R₁ is para-substituted wherein the substituent is selected from F, CF₃, Me, OMe, or isopropyl.

In one embodiment, the compound of formula VIa is

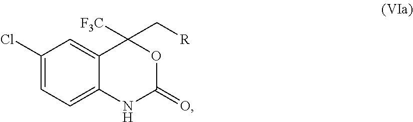

wherein R is selected from

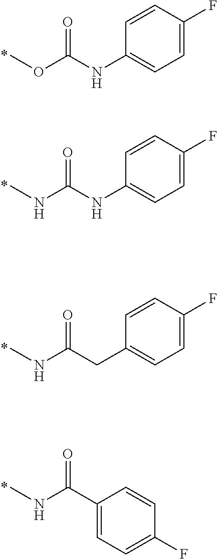

In another embodiment, the elongase inhibitor is a compound of formula VIb

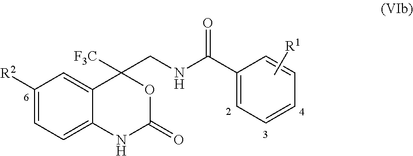

wherein R¹ is substituted at position 2, 3, or 4 with F, or Me, or R¹ is substituted at position 4 with MeO, or CF₃. R² is Cl, H, Ph, 4-isoxazol, 4-pyrazol, 3-pyrazol, 1-pyrazol, 5-(1,2,4-triazol), 1-(1,2,4-triazol), 2-imidazol, 1-(2-pyrrolidone), or 3-(1,3-oxazolidin-2-one). In one embodiment the compound of formula VI is

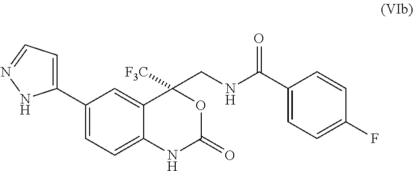 [SMILES: O=C1Nc2ccc(-c3ccn[nH]3)cc2[C@](CNC(=O)c2ccc(F)cc2)(C(F)(F)F)O1]

(S)-y. (See, Mizutani et al., 2009, J. Med. Chem. 52:7289-7300).

In another embodiment, the compound of formula VI is

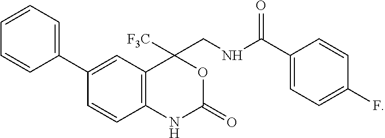 [SMILES: O=C1Nc2ccc(-c3ccccc3)cc2C(CNC(=O)c2ccc(F)cc2)(C(F)(F)F)O1]

Additional examples of an elongase inhibitors are compounds of formula VIIa and VIIb

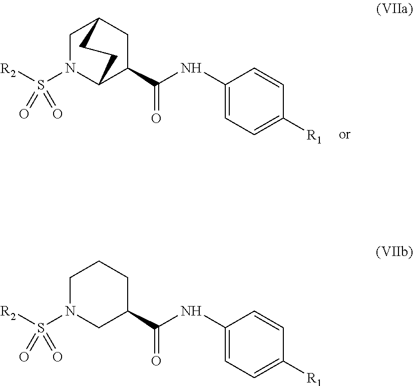

wherein R₁ is selected from OMe, OiPr, OCF₃, OPh, CH₂Ph, F, CH₃, CF₃, and benzyl; and

wherein R₂ is selected from C₁₋₄ alkyl (such as nBu, nPr, and iPr); phenyl; substituted phenyl where substitutents are selected from OMe, CF₃, F, tBu, iPr and thio; 2-pyridine; 3-pyridine; and N-methy imidazole. (See, Sasaki et al., 2009, Biorg. Med. Chem. 17:5639-47).

In one embodiment, R₁ is selected from OiPr and OCF₃. In one embodiment R₂ is selected from nBu, unsubstituted phenyl, fluorophenyl and thiophenyl.

In one embodiment the inhibitor of formula VIIa is

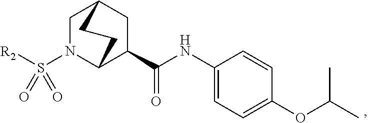

wherein R² is selected from butyl, propyl, phenyl, pyridyl, and imidazole.

In one embodiment the inhibitor of formula VIIa is selected from

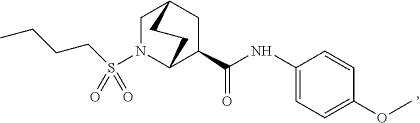 [SMILES: CCCCS(=O)(=O)N1C[C@H]2CC[C@@H]1[C@H](C(=O)Nc1ccc(OC)cc1)C2]

which has hELOVL6 IC₅₀ of 1710 nM;

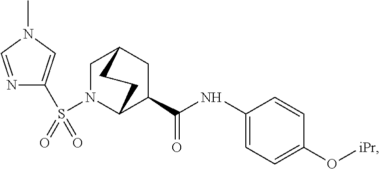 [SMILES: CC(C)Oc1ccc(NC(=O)[C@@H]2C[C@@H]3CC[C@H]2N(S(=O)(=O)c2cn(C)cn2)C3)cc1]

which has hELOVL6 IC₅₀ of 220 nM and a hELOVL3 IC₅₀ of 1510 nM; and

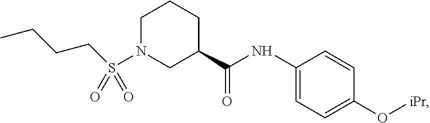 [SMILES: CCCCS(=O)(=O)N1CCC[C@@H](C(=O)Nc2ccc(OC(C)C)cc2)C1]

which has hELOVL6 IC₅₀ of 930 nM.

Yet another example of an elongase inhibitor is a compound of formula VIII

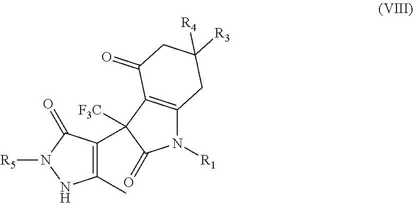

wherein R₁ is selected from H, unsubstituted phenyl; substituted phenyl where substitutents are selected from F, Me, Et, Cl, OMe, OCF₃, and CF₃; C₁₋₆ alkyl (such as Me, Et, iPr, and n-propyl); and C₃₋₆ cycloalkyl (cyclopropyl, cyclobutyl, cyclopentyl, cyclohexyl);
wherein R₃ and R₄ are independently selected from H; C₁₋₃ alkyl; and phenyl; or R₃ and R₄ taken together form a cycloalkyl of formula —(CH2)ₙ— where n=2, 3, 4 and 5;
wherein R₅ is selected from methyl; CF₃; cyclopropyl; unsubstituted phenyl; mono- and disubstituted phenyl where substitutents are selected from F, Me, Et, CN, iPr, Cl, OMe, OPh, OCF₃, and CF₃; unsubstituted heteroaromatic groups (such as 2, 3, or 4-pyridine, isoxazol, pyrazol, triazol); and imidazolo.

In other embodiments the compound of formula VIII is

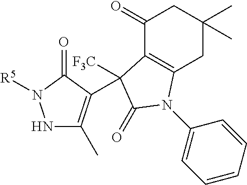

wherein R⁵ is a substituted phenyl ring, including, but not limited to

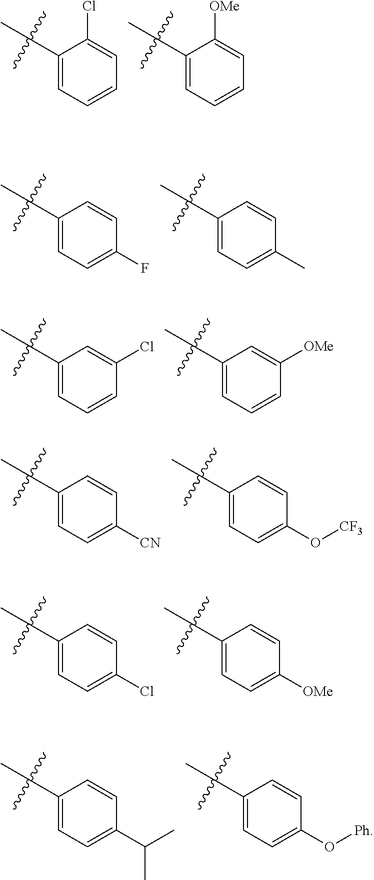

(See Takahashi et al., 2009, J. Med. Chem. 52:3142-5.)

In other embodiments a compound of formula VIII is one of the following compounds:

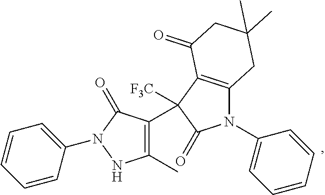 [SMILES: Cc1[nH]n(-c2ccccc2)c(=O)c1C1(C(F)(F)F)C(=O)N(c2ccccc2)C2=C1C(=O)CC(C)(C)C2]

which has a hELOVL6 IC₅₀ of 290 nM,

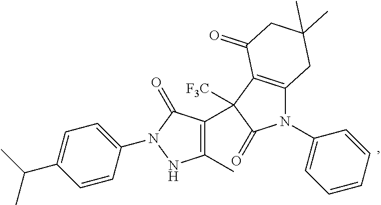 [SMILES: Cc1[nH]n(-c2ccc(C(C)C)cc2)c(=O)c1C1(C(F)(F)F)C(=O)N(c2ccccc2)C2=C1C(=O)CC(C)(C)C2]

which has a hELOVL6 IC₅₀ of 10 nM and a hELOVL3 IC₅₀ of 59 nM, and

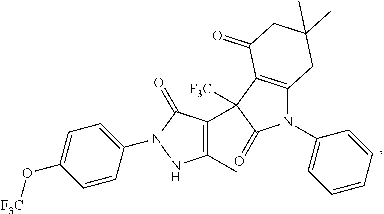 [SMILES: Cc1[nH]n(-c2ccc(OC(F)(F)F)cc2)c(=O)c1C1(C(F)(F)F)C(=O)N(c2ccccc2)C2=C1C(=O)CC(C)(C)C2]

Compound 37, which has a hELOVL6 IC₅₀ of 8.9 nM and a hELOVL3 IC₅₀ of 337 nM.

In one embodiment the elongase inhibitor is a compound of formula IX

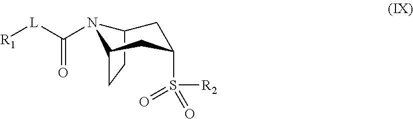

wherein L is selected from urea or an amide, for example

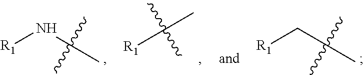

wherein R₁ is selected form 2-, 3-, and 4-pyridine; pyrimidine; unsubstituted heteroaryls such as isoxazol, pyrazol, triazol, imidazole; and unsubstituted phenyl; ortho, meta or para-substituted phenyl where substitutents are F, Me, Et, Cl, OMe, OCF₃, and CF₃, Cl, iPr and phenyl;
wherein R₂ is selected from Cl; iPr; phenyl; ortho, meta or para-substituted phenyl where substitutents are F, Me, Et, Cl, OMe, OCF₃, and CF₃; and heteroaryls such as 2-, 3-, and 4-pyridine, pyrimidine, and isoxazol, pyrazol, triazol, and imidazo.

In one embodiment L is urea. In one embodiment, R₁ is para-substituted CF₃ phenyl. In one embodiment, R₂ is phenyl. In another embodiment, R₂ is 2-pyridyl.

In one embodiment the compound of formula IX is selected from

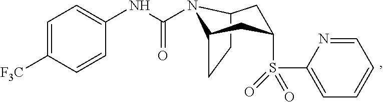

(endo-1w) which has a hELOVL6 IC₅₀ of 79 nM and a hELOVL3 IC50 of 6940 nM, and

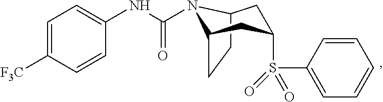

(endo-1k) which has a hELOVL6 IC50 of 78 nM.

1.2.5 ART1 Inhibitors

Meta-iodo-benzylguanidine (MIBG) is an inhibitor of ADP-ribosyltransferase 1 (ART1). 50 μM MIBG reduced HCMV titer from infected MRCS fibroblasts by about 70% with little or no effect on cell morphology.

1.2.6 AGXT2 Inhibitors

Aminooxyacetic acid (AOAA) is an inhibitor of alanine-glyoxylate aminotransferase 2 (AGXT2). 0.5 mM AOAA decreases HCMV replication by 100-fold with no measurable decrease in cell viability at concentrations up to 2.5 mM. 0.5 mM and 1 mM AOAA decreases influenza A replication in MDCK cells by at least 1000-fold after 24 hours with no evidence of host cell toxicity. 0.5 mM and 1 mM concentrations of AOAA decrease adenovirus titer in MRC2 cells by 20-fold and 500-fold respectively.

1.2.7 ACC Inhibitors

1.2.7.1 TOFA and its Analogs

TOFA (5-(tetradecyloxy)-2-furoic acid), an inhibitor of acetyl CoA carboxylase (ACC), is remarkably benign in mammals, see e.g., Gibson et al., Toxicity and teratogenicity studies with the hypolipidemic drug RMI 14,514 in rats. Fundam. Appl. Toxicol. 1981 January-February; 1(1):19-25. For example, in rats, the oral LD50 of TOFA can be greater than 5,000 mg/kg and no adverse effects are observed at 100 mg/kg/day for 6 months. In addition, TOFA is not teratogenic in rats at 150 mg/kg/day. ACC exists as two isozymes in humans, ACC1 and ACC2. Compounds described herein include, but are not limited to isozyme specific inhibitors of ACC. Non-limiting examples of ACC inhibitors include:

a Compound has the following structure (formula XI):

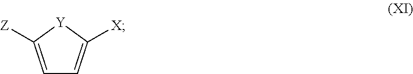

wherein:

Y is O or S; —NH or N(C₁-C₆)alky,

X is —COOH, —CO₂(C₁-C₆)alkyl, —CONH₂, —H, —CO(C₁-C₆)alkyl, —COC(halo)₃, a 5- or 6-membered heterocyclic ring having 1-3 heteroatoms selected from O, N, and S,

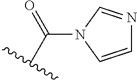 [SMILES: *C(=O)n1ccnc1]

or a moiety that can form an adduct with coenzyme A; and

Z is —(C₅-C₂₀)alkyl, —O(C₅-C₂₀)alkyl or —(C₅-C₂₀)alkoxy, —(C₅-C₂₀)haloalkyl, —O—(C₅-C₂₀)haloalkyl or —(C₅-C₂₀)haloalkoxy, -halo, —OH, —(C₅-C₂₀)alkenyl, —(C₅-C₂₀)alkynyl, —(C₅-C₂₀)alkoxy-alkenyl, —(C₅-C₂₀)hydroxyalkyl, —O(C₁-C₆)alkyl, —CO₂(C₁-C₆)alkyl, —O(C₅-C₂₀)alkenyl, —O(C₅-C₂₀)alkynyl, —O(C₅-C₂₀)cycloalkyl; —S(C₅-C₂₀)alkyl, —NH(C₅-C₂₀)alkyl, —NHCO(C₅-C₂₀)alkyl, —N(C₁-C₆)alkylCO(C₅-C₂₀)alkyl or —O(C₅-C₂₀)alkoxy.

In one embodiment, compounds of structure (XI) are those wherein Y is O.

In another embodiment, compounds of structure (XI) are those wherein X is —COOH.

In one embodiment, compounds of structure (XI) are those wherein X is selected from oxazole, oxadiazole, and

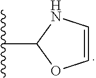 [SMILES: *C1NC=CO1]

In another embodiment, compounds of structure (XI) are those wherein Z is —O(C₅-C₂₀)alkyl, —O(C₅-C₂₀)haloalkyl, —O(C₅-C₂₀)alkenyl, —O(C₅-C₂₀)alkynyl or —O(C₅-C₂₀)alkoxy.

In another embodiment, compounds of structure (XI) are those wherein Y is O, X is —COOH and Z is —O(C₅-C₂₀)alkyl, —O(C₅-C₂₀)haloalkyl, —O(C₅-C₂₀)alkenyl, —O(C₅-C₂₀)alkynyl or —O(C₅-C₂₀)alkoxy.

In another embodiment, compounds of structure (XI) are those wherein X is a moiety that can form an ester linkage with coenzyme A. For example, X can be a moiety that allows for the formation of compounds of the structure:

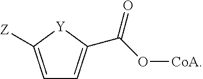

In a specific embodiment, a compound of structure (XI) is:

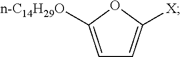

wherein:

X is —COOH, —CO₂(C₁-C₆)alkyl, —CONH₂, —H, —CO(C₁-C₆)alkyl, —COC(halo)₃,

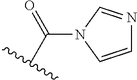 [SMILES: *C(=O)n1ccnc1]

or a moiety that can form an adduct with coenzyme A.

In another specific embodiment, a compound of structure (XI) is:

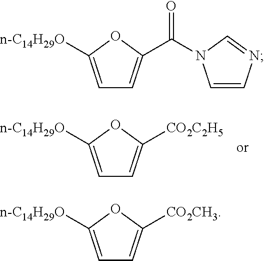

In a specific embodiment, the compounds of structure (XI) are the compounds disclosed in Parker et al., J. Med. Chem. 1977, 20, 781-791, which is herein incorporated by reference in its entirety.

In one embodiment, a Compound has the following structure (XII):

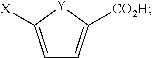

wherein:

X is —(C₅-C₂₀)alkyl, —O(C₅-C₂₀)alkyl, —(C₅-C₂₀)haloalkyl, —O(C₅-C₂₀)haloalkyl, -halo, —OH, —(C₅-C₂₀)alkenyl, —(C₅-C₂₀)alkynyl, —(C₅-C₂₀)alkoxy-alkenyl, —(C₅-C₂₀)hydroxyalkyl, —O(C₁-C₆)alkyl, —CO₂(C₁-C₆)alkyl, —O(C₅-C₂₀)alkenyl, —O(C₅-C₂₀)alkynyl, —O(C₅-C₂₀)cycloalkyl, —S(C₅-C₂₀)alkyl, —NH(C₅-C₂₀)alkyl, —NHCO(C₅-C₂₀)alkyl, —N(C₁-C₆)alkylCO(C₅-C₂₀)alkyl or —O(C₅-C₂₀)alkoxy;

Y is O, S, —NH or N(C₁-C₆)alkyl.

In a specific embodiment, a compound of structure (XII) is selected from:

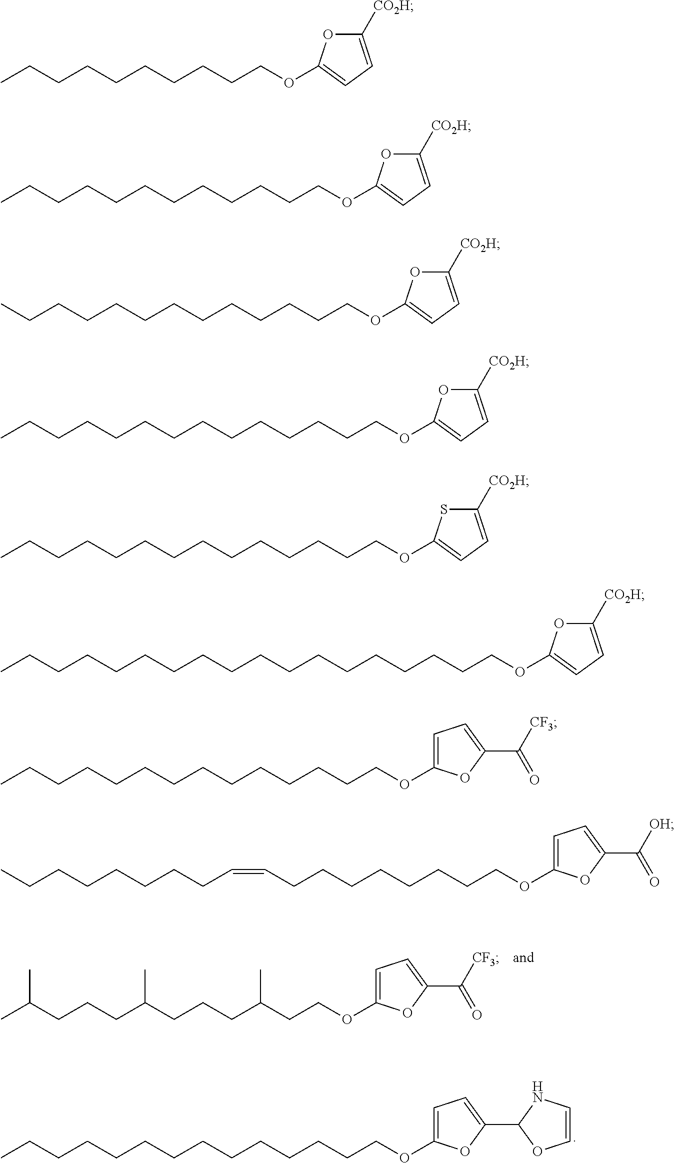

In a specific embodiment, the compounds of structure (XII) are the compounds disclosed in Parker et al., J. Med. Chem. 1977, 20, 781-791, which is herein incorporated by reference in its entirety.

In one embodiment, a compound of structure (XI) is:

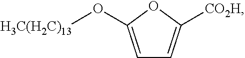

also referred to as TOFA and has the chemical name 5-(tetradecyloxy)-2-furoic acid.

1.2.7.2 Other ACC Inhibitors

In one embodiment, the ACC inhibitor is a compound with the structure (XIII) as follows:

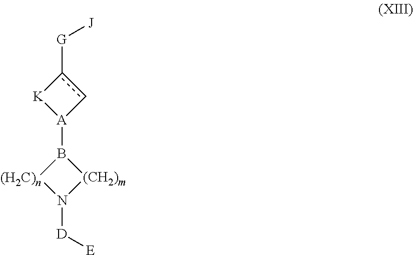

wherein A-B is N—CH or CH—N; K is (CH₂)ᵣ wherein r is 2, 3 or 4; m and n are each independently 1, 2 or 3 when A-B is N—CH or m and n are each independently 2 or 3 when A-B is CH—N; the dashed line represents the presence of an optional double bond;

D is carbonyl or sulfonyl;

E is either a) a bicyclic ring consisting of two fused fully unsaturated five to seven membered rings, taken independently, each of said rings optionally having one to four heteroatoms selected independently from oxygen, sulfur and nitrogen, or b) a tricyclic ring consisting of two fused fully unsaturated five to seven membered rings, taken independently, each of said rings optionally having one to four heteroatoms selected independently from oxygen, sulfur and nitrogen, said two fused rings fused to a third partially saturated, fully unsaturated or fully saturated five to seven membered ring, said third ring optionally having one to four heteroatoms selected independently from oxygen, sulfur and nitrogen; or c) a tetracyclic ring comprising a bicyclic ring consisting of two fused fully unsaturated five to seven membered rings, taken independently, each of said rings optionally having one to four heteroatoms selected independently from oxygen, sulfur and nitrogen, said bicyclic ring fused to two fully saturated, partially saturated or fully unsaturated five to seven membered monocyclic rings taken independently, each of said rings optionally having one to four heteroatoms selected independently from oxygen, sulfur and nitrogen or said bicyclic ring fused to a second bicyclic ring consisting of two fused fully saturated, partially saturated or fully unsaturated five to seven membered rings, taken independently, each of said rings optionally having one to four heteroatoms selected independently from oxygen, sulfur and nitrogen; or d) a teraryl ring comprising a fully unsaturated five to seven membered ring, said ring optionally having one to four heteroatoms selected independently from oxygen, sulfur and nitrogen, and said ring di-substituted independently with a fully unsaturated five to seven membered ring to form a teraryl nonfused ring system, each of said substituent rings optionally having one to four heteroatoms selected independently from oxygen, sulfur and nitrogen, wherein said E bi-, tri- or tetra cyclic ring or teraryl ring is optionally mono-, di- or tri-substituted independently on each ring used to form the bi-, tri- or tetra cyclic ring or teraryl ring with halo, hydroxy, amino, cyano, nitro, oxo, carboxy, (C₁-C₆) alkyl, (C₂-C₆) alkenyl, (C₂-C₆) alkynyl, (C₁-C₆) alkoxy, (C₁-C₄) alkylthio, (C₁-C₆) alkoxycarbonyl;

wherein said E bi-, tri- or tetra-cyclic ring or teraryl ring is optionally mono-substituted with a partially saturated, fully saturated or fully unsaturated three to eight membered ring R₁₀ optionally having one to four heteroatoms selected independently from oxygen, sulfur and nitrogen or a bicyclic ring R″ consisting of two fused partially saturated, fully saturated or fully unsaturated three to eight membered rings, taken independently, each of said rings optionally having one to four heteroatoms selected independently from oxygen, sulfur and nitrogen, said R₁₀ and R″ rings optionally additionally bridged and said R₁₀ and R″ rings optionally linked through a fully saturated, partially unsaturated or fully unsaturated one to four membered straight or branched carbon chain wherein the carbon (s) may optionally be replaced with one or two heteroatoms selected independently from oxygen, nitrogen and sulfur, provided said E bicyclic ring has at least one substituent and the E bicyclic ring atom bonded to D is carbon; wherein said R₁₀ or R″ring is optionally mono-, di- or tri-substituted independently with halo, hydroxy, amino, cyano, nitro, oxo, carboxy, (C₁-C₆) alkyl, (C₂-C₆) alkenyl, (C₂-C₆) alkynyl, (C₁-C₆) alkoxy, (C₁-C₄)alkylthio, (C₁-C₆) alkoxycarbonyl, (C₁-C₆) alkylcarbonyl, (C₁-C₆) alkylcarbonylamino, or mono-N— or di-N,N—(C₁-C₆) alkylamino or mono-N— or di-N,N—(C₁-C₆) alkylaminocarbonyl wherein said (C₁-C₆) alkyl and (C₁-C₆) alkoxy substituents are also optionally mono-, di- or tri-substituted independently with halo, hydroxy, (C₁-C₆) alkoxy, amino, mono-N— or di-N,N—(C₁-C₆) alkylamino or from one to nine fluorines;

G is carbonyl, sulfonyl or CR₇R₈; wherein R₇ and R₈ are each independently H, (C₁-C₆) alkyl, (C₂-C₆) alkenyl or (C₂-C₆) alkynyl or a five to seven membered partially saturated, fully saturated or fully unsaturated ring optionally having one heteroatom selected from oxygen, sulfur and nitrogen;

J is OR′, NR₂R₃ or CR₄R₅R₆; wherein R′, R₂ and R₃ are each independently H, Q, or a (C₁-C₁₀) alkyl, (C₃-C₁₀) alkenyl or (C₃-C₁₀) alkynyl substituent wherein said carbon(s) may optionally be replaced with one or two heteroatoms selected independently from oxygen, nitrogen and sulfur and wherein said sulfur is optionally mono- or di-substituted with oxo, said carbon (s) is optionally mono-substituted with oxo, said nitrogen is optionally di-substituted with oxo, said carbon (s) is optionally mono-, di- or tri-substituted in dependently with halo, hydroxy, amino, nitro, cyano, carboxy, (C₁-C₄) alkylthio, (C₁-C₆)alkyloxycarbonyl, mono-N— or di-N,N—(C₁-C₆) alkylamino or mono-N— or di-N,N—(C₁-C₆)alkylaminocarbonyl; and said chain is optionally mono-substituted with Q₁; wherein Q and Q₁ are each independently a partially saturated, fully saturated or fully unsaturated three to eight membered ring optionally having one to three heteroatoms selected independently from oxygen, sulfur and nitrogen or a bicyclic ring consisting of two fused or spirocyclic partially saturated, fully saturated or fully unsaturated three to six membered rings, taken independently, said bicyclic ring optionally having one to three heteroatoms selected independently from oxygen, sulfur and nitrogen, said mono or bicyclic ring optionally additionally bridged with (C₁-C₃) alkylen wherein said (C₁-C₃) alkylen carbons are optionally replaced with one to two heteroatoms selected independently from oxygen, sulfur and nitrogen; wherein said Q and Q₁ ring are each independently optionally mono-, di-, tri-, or tetra-substituted independently with halo, hydroxy, amino, nitro, cyano, oxo, carboxy, (C₁-C₆)alkyl, (C₂-C₆) alkenyl, (C₂-C₆) alkynyl, (C₁-C₆) alkoxy, (C₁-C₄) alkylthio, (C₁-C₆) alkylcarbonyl, (C₁-C₆) alkylcarbonylamino, (C₁-C₆)alkyloxycarbonyl, mono-N— or di-N,N—(C₁-C₆) alkylamino, mono-N— or di-N,N—(C₁-C₆)alkylaminosulfonyl, mono-N— or di-N,N—(C₁-C₆) alkylaminocarbonyl, wherein said (C₁-C₆) alkyl substituent is optionally mono-, di- or tri-substituted independently with halo, hydroxy, amino, nitro, cyano, oxo, carboxy, (C₁-C₆)alkoxy, (C₁-C₄) alkylthio, C₆)alkyloxycarbonyl or mono-N— or di-N,N—(C₁-C₆)alkylamino wherein said (C₁-C₆) alkyl substituent is also optionally substituted with from one to nine fluorines;

or wherein R₂ and R₃ can be taken together with the nitrogen atom to which they are attached to form a partially saturated, fully saturated or fully unsaturated three to eight membered ring optionally having one to three additional heteroatoms selected independently from oxygen, sulfur and nitrogen or a bicyclic ring consisting of two fused, bridged or spirocyclic partially saturated, fully saturated or fully unsaturated three to six membered rings, taken independently, said bicyclic ring optionally having one to three additional heteroatoms selected independently from oxygen, sulfur and nitrogen or a tricyclic ring consisting of three fused, bridged or spirocyclic partially saturated, fully saturated or fully unsaturated three to six membered rings, taken independently, said tricyclic ring optionally having one to three additional heteroatoms selected independently from oxygen, sulfur and nitrogen; wherein said NR₂R₃ ring is optionally mono-, di-, tri- or tetra-substituted independently with R15, halo, hydroxy, amino, nitro, cyano, oxo, carboxy, (C₁-C₆) alkyl, (C₂-C₆) alkenyl, (C₂-C₆) alkynyl, (C₁-C₆) alkoxy, (C₁-C₄) alkylthio, (C₁-C₆) alkylcarbonylamino or mono-N— or di-N,N—(C₁-C₆) alkylamino, wherein said (C₁-C₆) alkyl substituent is optionally mono-, di- or tri-substituted independently with halo, hydroxy, amino, nitro, cyano, oxo, carboxy, (C₁-C₆) alkoxy, (C₁-C₄) alkylthio, (C₁-C₆) alkyloxycarbonyl, mono-N— or di-N,N—(C₁-C₆) alkylamino, said (C₁-C₆) alkyl substituent is also optionally substituted with from one to nine fluorines;

wherein three heteroatoms selected independently from oxygen, sulfur and nitrogen wherein said ring is optionally mono-, di- or tri-substituted with halo, hydroxy, amino, nitro, cyano, oxo, carboxy, (C₁-C₆) alkyl, (C₂-C₆) alkenyl, (C₂-C₆)alkynyl, (C₁-C₄)alkylthio, (C₁-C₆) alkoxy, (C₁-C₆)alkylcarbonylamino, mono-N— or di-N,N—(C₁-C₆) alkylamino; wherein said NR₂R₃ ring is optionally substituted with a partially saturated, fully saturated or fully unsaturated three to eight membered ring optionally having one to three heteroatoms selected independently from oxygen, sulfur and nitrogen or a bicyclic ring consisting of two fused partially saturated, fully saturated or fully unsaturated three to six membered rings, taken independently, said bicyclic ring optionally having one to three heteroatoms selected independently from oxygen, sulfur and nitrogen, said mono or bicyclic ring optionally additionally bridged said ring optionally having one to three heteroatoms selected independently from oxygen, sulfur and nitrogen, wherein said (C₁-C₆) alkyl and said ring are optionally mono-, di- or tri-substituted with halo, hydroxy, amino, nitro, cyano, oxo, carboxy, (C₂-C₆) alkenyl, (C₃-C₆) alkynyl, (C₁-C₆) alkylcarbonylamino, hydroxy, (C₁-C₆) alkoxy, (C₁-C₄) alkylthio, (C₁-C₆) alkoxy, mono-N— or di-N,N—(C₁-C₆) alkylamino; wherein R₄, R₅ and R₆ are independently H, halo, hydroxy, (C₁-C₆) alkyl or R₄ and R₅ are taken together to form a partially saturated, fully saturated or fully unsaturated three to eight membered ring, said ring optionally having one to three heteroatoms selected independently from oxygen, sulfur and nitrogen, wherein said (C₁-C₆) alkyl and said ring are optionally mono-, di- or tri-substituted with halo, hydroxy, amino, nitro, cyano, oxo, carboxy, (C₂-C₆) alkenyl, (C₃-C₆) alkynyl, (C₁-C₆) alkylcarbonylamino, hydroxy, (C₁-C₆) alkoxy, (C₁-C₄) alkylthio, (C₁-C₆) alkoxy, mono-N— or di-N,N—(C₁-C₆) alkylamino with the proviso that 1′-(anthracene-9-carbonyl)-[1,4′]bipiperidinyl-3-carboxylic aciddiethyiamide; 1′-(1-oxa-2,3-diaza-cyclopenta[a]naphthalene-5-sulfonyl)-[1,4′]bipiperidinyl-3 carboxylic acid diethylamide; 1′-(5-dimethylamino-naphthalene-1-sulfonyl)-[1,4′]bipiperidinyl-3-carboxylic acid diethylamide; 1′-(9,10,10-trioxo-9,10-dihydro-thioxanthene-3-carbonyl)-[1,4′]bipiperidinyl-3-carboxylic acid diethylamide; and 1′-(2-Oxo-2H-chromen-3-carbonyl)-[1,4′]bipiperidinyl-3-carboxylic acid diethylamide are not included.

Compounds of structure (XIII) can be made using organic synthesis techniques known to those skilled in the art, as well as by the methods described in (International Patent Publication WO 03/072197), which is incorporated herein by reference in its entirety (particularly at page 103, line 14 to page 160, line 17). Further, specific examples of these compounds can be found in this publication.

Other specific examples of compounds of structure (XIII) are:

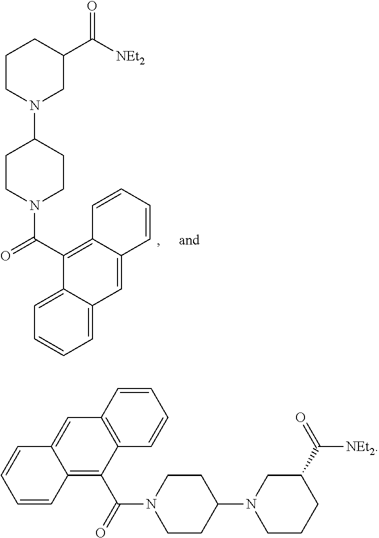

also known as CP-610431.

Other specific examples of compounds of structure (XIII) are:

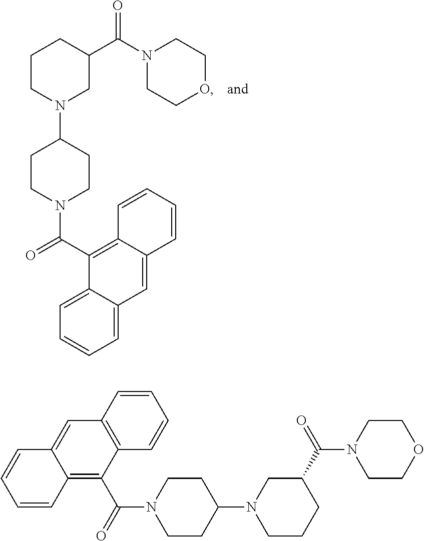

also known as CP-640186.

In another embodiment a compound of structure (XIII) is:

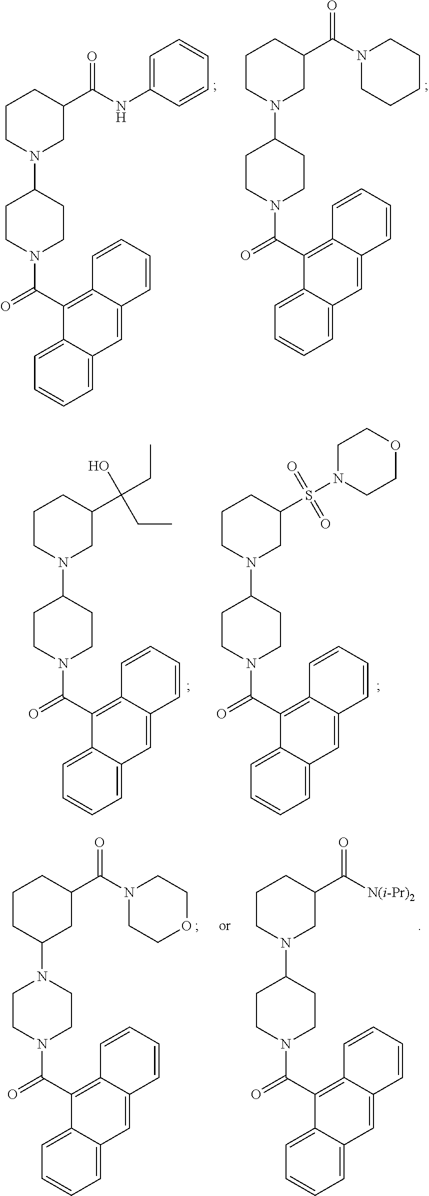

In one embodiment, the compound of structure (XIII) is not CP-610431.

In another embodiment, the compound of structure (XIII) is not CP-640186.

In one embodiment, the ACC inhibitor is a compound with the structure (XIV) as follows:

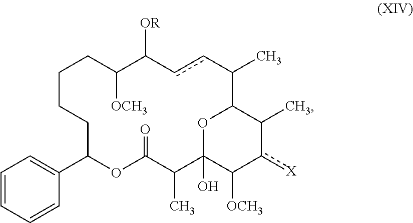

In this formula, the dotted lines are independently a saturated bond or a double bond, alternatively, while R is hydrogen, CH₃ or —C(O)A, where A is hydrogen, (C₃-C₆)cycloalkyl or (C₁-C₆)alkyl which is unsubstituted or substituted by halogen or (C₁-C₃)alkoxy, and

X is —OH if the bond is saturated, or ═O, ═N—OY or ═N—N(R₁)(R₂) if there is an unsaturated bond, where

Y is hydrogen, (C₁-C₆)alkyl, (C₃-C₆)alkenyl, (C₃-C₆)alkynyl or an acyl group —C(O)—Z in which

Z is phenyl, or a (C₁-C₆)alkyl group which is substituted by halogen or (C₁-C₄)alkoxy, or is hydrogen, (C₁-C₆)alkyl, (C₂-C₆)alkenyl or (C₂-C₆)alkynyl;

R₁ is hydrogen or (C₁-C₆)alkyl and

R₂ is hydrogen, (C₁-C₆)alkyl, phenyl, carbamoyl(CONH₂), —COA or —SO₂—R₃, where

R₃ is (C₁-C₆) alkyl, or is phenyl which is unsubstituted or substituted by (C₁-C₄)alkyl.

Compounds of structure (XIV) can be made using organic synthesis techniques known to those skilled in the art, as well as by the methods described in Bohlendorf et. al. (U.S. Pat. No. 5,026,878), which is incorporated herein by reference in its entirety (particularly at column 10, line 25 to column 16, line 14). Further, specific examples of these compounds can be found in this publication.

A specific example of a compound of structure (XIV) is:

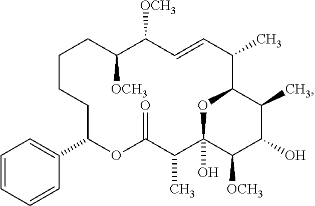

which is also known as Soraphen A.

In a particular embodiment a compound of structure (XIV) is:

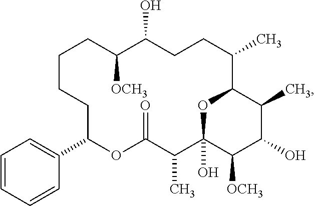

which is also known as Soraphen B.

In another embodiment a compound of structure (XIV) is:

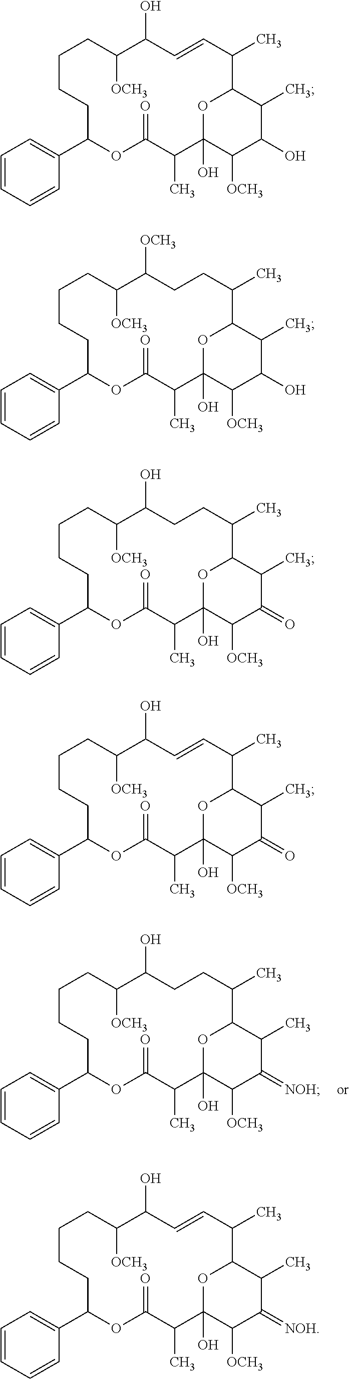

In one embodiment, the compound of structure (XIV) is not Soraphen A.

In one embodiment, the compound of structure (XIV) is not Soraphen B.

In one embodiment, the modulator of a host cell target enzyme is an ACC inhibitor of (XV) as follows:

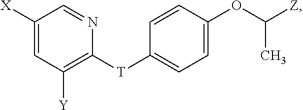

wherein T is oxygen or sulfur;
X is Cl, Br or CF₃;
Y is H, Cl, Br or CF₃, provided at least one of X and Y is CF₃;
Z is —C(O)OR₁, —C(O)NR₂R₃, —C(O)O⁻M⁺, —C(O)SR₄, —CNR₁ is H, (C₁-C₈)alkyl, benzyl, chlorobenzyl or C₃-C₆ alkoxyalkyl;
R₄ is (C₁-C₄)alkyl;
R₅ is H or (C₁-C₄) alkyl;
R₆ is (C₁-C₇) alkyl;
M is NHR₂R₃R₇, Na, K, Mg or Ca;
R₂ and R₃ are each independently selected from R₇ or —OCH₃, provided both R₂ and R₃ cannot be simultaneously —OCH₃ and neither is —OCH₃ in —NHR₂R₃R₇; and
R₇ is H, (C₁-C₄)alkyl or (C₂-C₃)hydroxyalkyl.

A specific example of a compound of structure (XV) is:

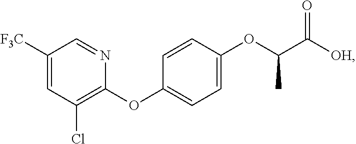 [SMILES: C[C@@H](Oc1ccc(Oc2ncc(C(F)(F)F)cc2Cl)cc1)C(=O)O]

which is also known as haloxyfop.

In another embodiment a compound of structure (XV) is:

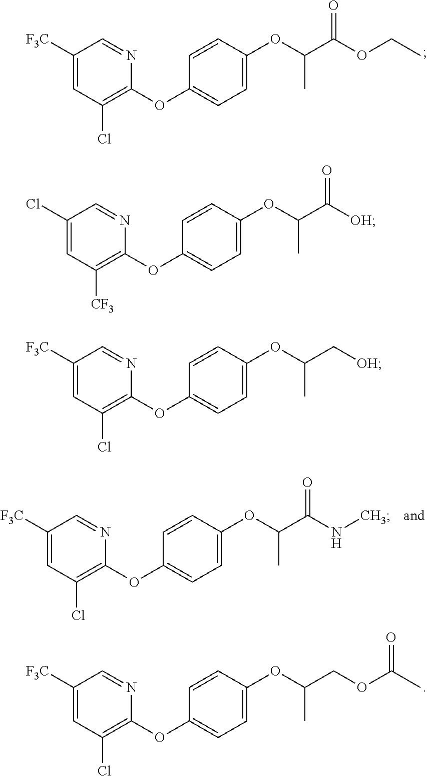

In one embodiment, the compound of structure (XV) is not haloxyfop.

In one embodiment, the modulator of the host cell target enzyme is a compound with the following structure (XVI):

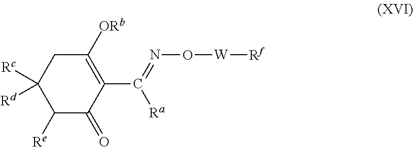

wherein:
R^{a} is C₁-C₆-alkyl;
R^{b} is hydrogen, one equivalent of an agriculturally useful cation, C₂-C₈-alkylcarbonyloxy, C₁-C₁₀-alkylsulfonyl, C₁-C₁₀-alkylphosphonyl or benzoyl, benzenesulfonyl or benzenephosphonyl, where the three last-mentioned groups may furthermore each carry from one to five halogen atoms;
R^{c} is hydrogen, cyano, formyl, C₁-C₆ allyl, C₁-C₄-alkoxy-C₁-C₆-alkyl or C₁-C₄-alkylthio-C₁-C₆-alkyl, phenoxy-C₁-C₆-alkyl, phenylthio-pyridyloxy-C₁-C₆-alkyl or pyridylthio-C₁-C₆-alkyl, where the phenyl and pyridyl rings may each furthermore carry from one to three radicals selected from the group consisting of nitro, cyano, halogen, C₁-C₄-alkyl, partially or completely halogenated C₁-C₄-alkyl, C₁-C₄-alkoxy, partially or completely halogenated C₁-C₄-alkoxy, C₁-C₄-alkylthio, C₃-C₆-alkenyl, C₃-C₆-alkenyloxy, C₃-C₆-alkynyl, C₃-C₆-alkynyloxy and —NR^{g}R^{h}, where
R^{g} is hydrogen, C₁-C₄-alkyl, C₃-C₆-alkenyl, C₃-C₆-alkynyl, C₁-C₆-acyl or benzoyl which may carry from one to three radicals selected from the group consisting of nitro, cyano, halogen, C₁-C₄-alkyl, partially or completely halogenated C₁-C₄-alkyl, C₁-C₄-alkoxy and C₁-C₄-alkylthio and
R^{h} is hydrogen, C₁-C₄-alkyl, C₃-C₆-alkenyl or C₃-C₆-alkynyl; C₃-C₇-cycloalkyl or C₅-C₇-cycloalkenyl, where these groups may furthermore carry from one to three radicals selected from the group consisting of hydroxyl, halogen, C₁-C₄-alkyl, partially or completely halogenated C₁-C₄-alkyl, C₁-C₄-alkoxy, C₁-C₄-alkylthio, benzylthio, C₁-C₄-alkylsulfonyl, C₁-C₄-alkylsulfenyl and C₁-C₄-alkylsulfinyl, a 5-membered saturated heterocyclic structure which contains one or two oxygen or sulfur atoms or one oxygen and one sulfur atom as hetero atoms and which may furthermore carry from one to three radicals selected from the group consisting of C₁-C₄-alkyl, partially or completely halogenated C₁-C₄-alkyl, C₁-C₄-alkoxy and C₁-C₄-alkylthio, a 6-membered or 7-membered saturated heterocyclic structure or mono- or diunsaturated heterocyclic structure which contains one or two oxygen or sulfur atoms or one oxygen and one sulfur atom as hetero atoms and which may furthermore carry from one to three radicals selected from the group consisting of hydroxyl, halogen, C₁-C₄-alkyl, partially or completely halogenated C₁-C₄-alkyl, C₁-C₄-alkoxy and C₁-C₄-alkylthio, a 5-membered heteroaromatic structure containing from one to three hetero atoms selected from the group consisting of one or two nitrogen atoms and one oxygen or sulfur atom, where the heteroaromatic structure may furthermore carry from one to three radicals selected from a group consisting of cyano, halogen, C₁-C₄-alkyl, partially or completely halogenated C₁-C₄-alkyl, C₁-C₄-alkoxy, partially or completely halogenated C₁-C₄-alkoxy, C₁-C₄-alkylthio, C₂-C₆-alkenyl, C₂-C₆-alkenyloxy, C₃-C₆-alkynyloxy and C₁-C₄-alkoxy-C₁-C₄-alkyl, phenyl or pyridyl, each of which may furthermore carry from one to three radicals selected from the group consisting of nitro, cyano, formyl, halogen, C₁-C₄-alkyl, partially or completely halogenated C₁-C₄-alkyl, C₁-C₄-alkoxy, partially or completely halogenated C₁-C₄-alkoxy, C₁-C₄-alkylthio, C₃-C₆-alkenyl, C₃-C₆-alkenyloxy, C₃-C₆-alkynyl, C₃-C₆-alkynyloxy and —NR^{g}R^{h}, where R^{g} and R^{h} have the abovementioned meanings;
R^{d} is hydrogen, hydroxyl or C₁-C₆-alkyl;
R^{o} is hydrogen, halogen, cyano, a C₁-C₄-alkoxycarbonyl or a C₁-C₄-alkylketoxime group;
W is a C₁-C₆-alkylene, C₃-C₆-alkenylene or C₃-C₆-alkynylene chain, each of which may furthermore carry from one to three radicals selected from the group consisting of three C₃-C₆-alkyl substituents, three halogen atoms and one methylene substituent; a C₃-C₆-alkylene or C₄-C₆-alkenylene chain, both of which may furthermore carry from one to three C₃-C₆-alkyl radicals, where in each case one methylene group of the chains may be replaced by an oxygen or sulfur atom, a sulfoxyl or sulfonyl group or a group —N(Rⁱ)—, where Rⁱ is hydrogen, C₁-C₄-alkyl, C₃-C₆-alkenyl or C₃-C₆-alkynyl;
R^{f} is hydrogen; C₁-C₆-alkyl; vinyl; a group —CH═CH—Z, where Z is cyano, halogen, C₁-C₄-alkyl, partially or completely halogenated C C₃-C₆-cycloalkyl, which, if desired, in turn may carry from one to three substituents selected from the group consisting of hydroxyl, halogen, C₁-C₄-alkyl, partially or completely halogenated C₁-C₄-alkyl and C₁-C₄-alkoxy; carboxyl, C₁-C₈-alkoxycarbonyl, benzyloxycarbonyl, phenyl, thienyl or pyridyl, where these three aromatic radicals may be unsubstituted or may carry from one to three substituents selected from the group consisting of nitro, cyano, halogen, C₁-C₄-alkyl, partially or completely halogenated C₁-C₄-alkyl, C₁-C₄-alkoxy, partially or completely halogenated C₁-C₄-alkoxy, C₁-C₄-alkylthio and C₃-C₆-cycloalkyl, where the cycloalkyl substituent may be unsubstituted or in turn may furthermore carry from one to three radicals selected from the group consisting of halogen, C₁-C₄-alkyl, partially or completely halogenated C₁-C₄-alkyl and C₁-C₄-alkoxy; ethynyl which may carry one of the following radicals: C₁-C₄-alkyl, C₃-C₆-cycloalkyl, which, if desired, may carry from one to three substituents selected from the group consisting of hydroxy, halogen, C₁-C₄-alkyl, partially or completely halogenated C₁-C₄-alkyl and C₁-C₄-alkoxy, or phenyl, thienyl or pyridyl, where these aromatic radicals may be unsubstituted or may each furthermore carry from one to three substituents selected from the group consisting of nitro, cyano, halogen, C₁-C₄-alkyl, partially or completely halogenated C C₁-C₄-alkoxy, partially or completely halogenated C₁-C₄-alkoxy and C₁-C₄-alkylthio; phenyl, halophenyl, dihalophenyl, a 5-membered heteroaromatic group having from one to three hetero atoms, selected from the group consisting of from one to three nitrogen atoms and one oxygen or sulfur atom, or a 6-membered heteroaromatic group having from one to four nitrogen atoms, all of which may not be adjacent to one another at the same time, where the phenyl and hetaryl groups may, if desired, furthermore carry from one to three radicals selected from the group consisting of nitro, C₁-C₄-alkoxy, C₁-C₄-alkylthio, partially or completely halogenated C₁-C₄-alkoxy, radicals Z and —NR^{k}R¹, where
R^{k} is hydrogen, C₁-C₄-alkyl, C₃-C₆-alkenyl or C₃-C₆-alkynyl; and
R^{l} is hydrogen, C₁-C₄-alkyl, C₃-C₆-alkenyl, C₃-C₆-alkynyl, C₁-C₆-acyl or benzoyl which, if desired, may furthermore carry from one to three substituents selected from the group consisting of nitro, cyano, halogen, C₁-C₄-alkyl, partially or completely halogenated C₁-C₄-alkyl, C₁-C₄-alkoxy and C₁-C₄-alkylthio.

Compounds of structure (XVI) can be made using organic synthesis techniques known to those skilled in the art, as well as by the methods described in U.S. Pat. No. 5,491,123, issued Feb. 13, 1996, which is incorporated herein by reference in its entirety (particularly at column 11, line 62 to column 13, line 5). Further, specific examples of these compounds can be found in this patent. Additional examples of compounds of structure (XVI) are found in U.S. Pat. No. 6,383,987, issued May 7, 2002; U.S. Pat. No. 6,103,664, issued Aug. 15, 2000; and U.S. Pat. No. 4,334,913, issued Jun. 15, 1982, each being incorporated herein by reference in its entirety.

A specific example of a compound of structure (XVI) is:

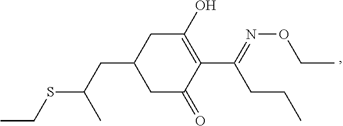 [SMILES: CCC/C(=N\OCC)C1=C(O)CC(CC(C)SCC)CC1=O]

which is also identified as sethoxydim.

In another embodiment, the compound of structure (XVI) is:

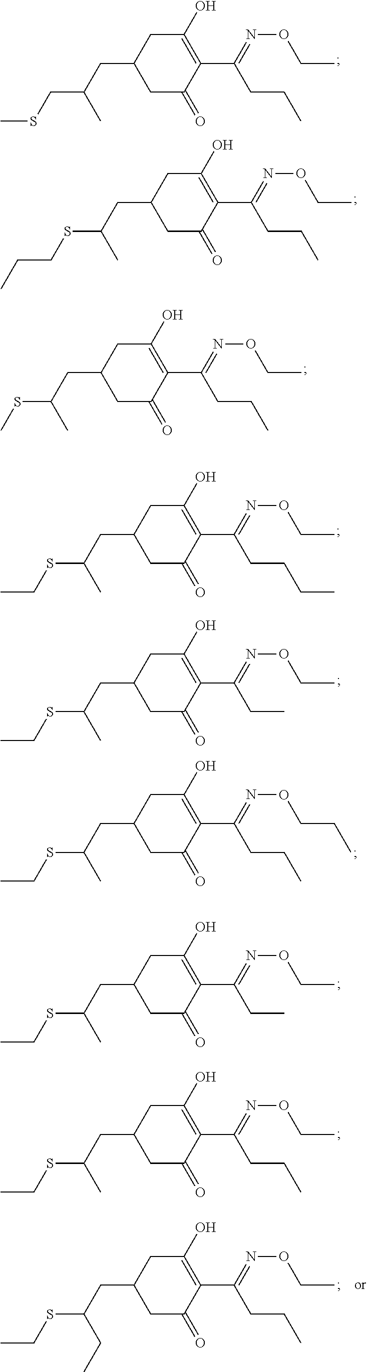

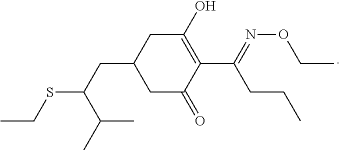 [SMILES: CCC/C(=N\OCC)C1=C(O)CC(CC(SCC)C(C)C)CC1=O]

In one embodiment, the compound of structure (XVI) is not sethoxydim.

In one embodiment, the modulator of a host cell target is a compound that is an inhibitor of ACC with the structure (XVII) as follows:

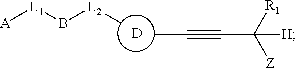

or therapeutically suitable salt, ester or prodrug, thereof, wherein:
A is selected from the group consisting of alkenyl, alkoxyalkyl, alkyl, aryl, arylalkyl, cycloalkyl, cycloalkylalkyl, haloalkyl, heteroaryl, heteroarylalkyl, heterocycle, and heterocyclealkyl;
B is selected from the group consisting of an aryl ring and a heteroaryl ring, which may optionally be substituted with halo, -halo, —OH, —NO₂, NHC(O)—(C₁₋₆)alkyl, CHO, vinyl, allyl, (C₁₋₆)hydroxyalkyl, NH₂, NH(C₁₋₆)alkyl, N[(C₁₋₆)alkyl]₂CH═NOH, CH₂N[(C₁₋₆)alkyl]₂ or CN;
D is selected from the group consisting of an aryl ring and a heteroaryl ring;
L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, (C₁₋₆)haloalkyl or (C₁₋₆)alkyl; Rₐ and R_{b} are each individually selected from the group consisting of hydrogen, alkyl, haloalkyl and hydroxy or Rₐ and R_{b} taken together with the atom to which they are attached form R_{f}—N═.;
R_{c} is selected from the group consisting of hydrogen, alkyl, aryl, haloalkyl, and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or (C₁₋₆)alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

An embodiment of structure (XVII), is structure (XVIIa):

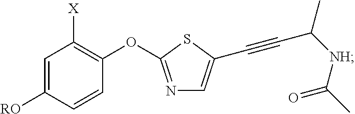

wherein R is (C₁₋₆)alkyl, (C₁₋₆)alkyl-cycloalkyl, (C₁₋₆)alkyl-heteroaryl, (C₁₋₆)alkyl-heterocycloalkyl; and wherein X is -halo, —OH, —NO₂, NHC(O)—(C₁₋₆)alkyl, CHO, vinyl, allyl, (C₁₋₆)hydroxyalkyl, NH₂, NH(C₁₋₆)alkyl, N[(C₁₋₆)alkyl]₂CH═NOH, CH₂N[(C₁₋₆)alkyl]₂ or CN;

Specific embodiments of structure (XVIIa) are presented in the table below:

| | |
|---|---|
| | XVIIa |
| 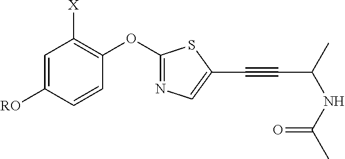 | |

| Compound | R | X |
|---|---|---|
| XIVa1 | i-Pr | H |
| XIVa2 | i-Bu | H |
| XIVa3 | Pr | H |
| XIVa4 | CH₂(cyclopropyl) | H |
| XIVa5 | Cyclohexyl | H |
| XIVa6 | CH₂(cyclohexyl) | H |
| XIVa7 | CH₂(Tetrahydrofuran-3-yl) | H |
| XIVa8 | i-Pr | Cl |
| XIVa9 | i-Bu | Cl |
| XIVa10 | Pr | Cl |
| XIVa11 | CH₂(cyclopropyl) | Cl |
| XIVa12 | Cyclohexyl | Cl |
| XIVa13 | CH₂(cyclohexyl) | Cl |
| XIVa14 | CH₂(Tetrahydrofuran-3-yl) | Cl |
| XIVa15 | i-Bu | F |
| XIVa16 | i-Bu | Br |
| XIVa17 | i-Bu | Me |
| XIVa18 | i-Bu | NO₂ |
| XIVa19 | i-Bu | NH₂ |
| XIVa20 | i-Bu | NHCOMe |
| XIVa21 | i-Bu | CHO |
| XIVa22 | i-Bu | CH═NOH |
| XIVa23 | i-Bu | CN |
| XIVa24 | i-Bu | Vinyl |
| XIVa25 | i-Bu | CH₂OH |
| XIVa26 | i-Bu | CH₂NMe₂ |

Another embodiment of structure (XVII), is structure (XVIIb):

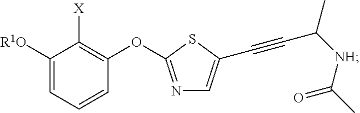

wherein: R is (C₁₋₆)alkyl, (C₁₋₆)alkyl-cycloalkyl, (C₁₋₆)alkyl-heteroaryl, (C₁₋₆)alkyl-heterocycloalkyl; and wherein X is -halo, —OH, —NO₂, NHC(O)—(C₁₋₆)alkyl, CHO, vinyl, allyl, (C₁₋₆)hydroxyalkyl, NH₂, NH(C₁₋₆)alkyl, N[(C₁₋₆)alkyl]₂CH═NOH, CH₂N[(C₁₋₆)alkyl]₂ or CN;

In a specific embodiment, the compound of structure (XVIIb) is:

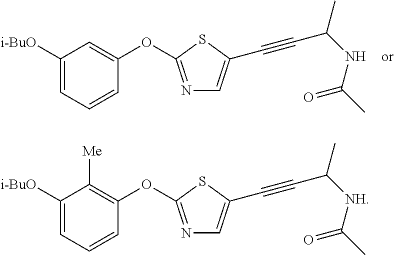

In specific embodiment, the compound of structure (XVII) is:

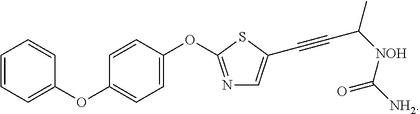

In specific embodiment, the compound of structure (XVII) is not:

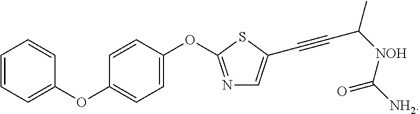

In one embodiment the compound of structure (XVII) is

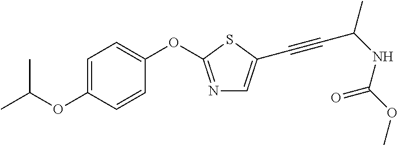 [SMILES: COC(=O)NC(C)C#Cc1cnc(Oc2ccc(OC(C)C)cc2)s1]

In one embodiment, the ACC inhibitor has the following structure:

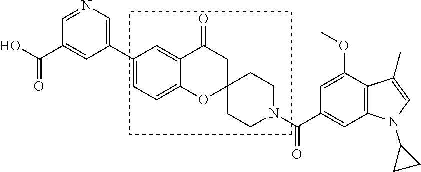 [SMILES: COc1cc(C(=O)N2CCC3(CC2)CC(=O)c2cc(-c4cncc(C(=O)O)c4)ccc2O3)cc2c1c(C)cn2C1CC1]

### (see WO08088688 and US2008171761), or

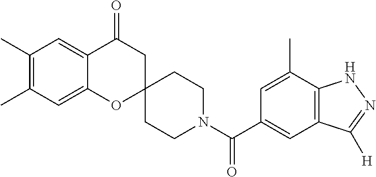 [SMILES: Cc1cc2c(cc1C)C(=O)CC1(CCN(C(=O)c3cc(C)c4[nH]ncc4c3)CC1)O2]

### (see WO08065508).

1.2.8 Fatty Acid Synthase (FAS) Inhibitors

In one embodiment, the modulator of a host cell target is an inhibitor of Fatty Acid Synthase (FAS). In one embodiment the FAS inhibitor has the following structure (XVIII):

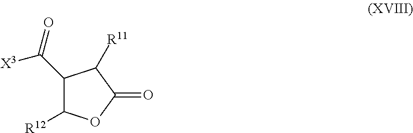

wherein:
R¹¹ is H, or C₁-C₂₀ alkyl, cycloalkyl, alkenyl, aryl, arylalkyl, or alkylaryl, ═CHR¹³, —C(O)OR¹³, —C(O)R¹³, —CH₂C(O)OR¹³, —CH₂C(O)NHR¹³, where R¹³ is H or C₁-C₁₀ alkyl, cycloalkyl, or alkenyl;
R₁₂ is C₁-C₂₀alkyl, cycloalkyl, alkenyl, aryl, arylalkyl, or alkylaryl;
X³ is OR¹⁴ or NHR¹⁴, where R¹⁴ is H, C₁-C₂₀ alkyl, hydroxyalkyl, cycloalkyl, alkenyl, aryl, arylalkyl, or alkylaryl, the R¹⁴ group optionally containing a carbonyl group, a carboxyl group, a carboxyamide group, an alcohol group, or an ether group, the R¹⁴ group further optionally containing one or more halogen atoms.

Compounds of structure (XVIII) can be made using organic synthesis techniques known to those skilled in the art, as well as by the methods described in U.S. Patent Application Publication No. 2006/0241177, published Oct. 26, 2006, which is incorporated herein by reference in its entirety (particularly at pages 7-10 and FIGS. 1 and 2). Further, specific examples of these compounds can be found in this publication. Additional examples of compounds of structure (XVIII) are found in International Patent Publication No. WO 2004/041189, published May 21, 2004; International Patent Publication No. WO 97/18806, published May 29, 1997; and U.S. Patent Application Publication No. 2005/0239877, published Oct. 27, 2005, each being incorporated herein by reference in its entirety.

A specific example of a compound of structure (XVIII) is:

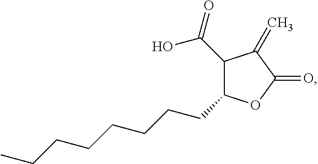 [SMILES: C=C1C(=O)O[C@H](CCCCCCCC)C1C(=O)O]

which is also identified as C75 (trans-4-carboxy-5-octyl-3-methylene-butyrolactone).

In another embodiment, the compound of structure (XVIII) is:

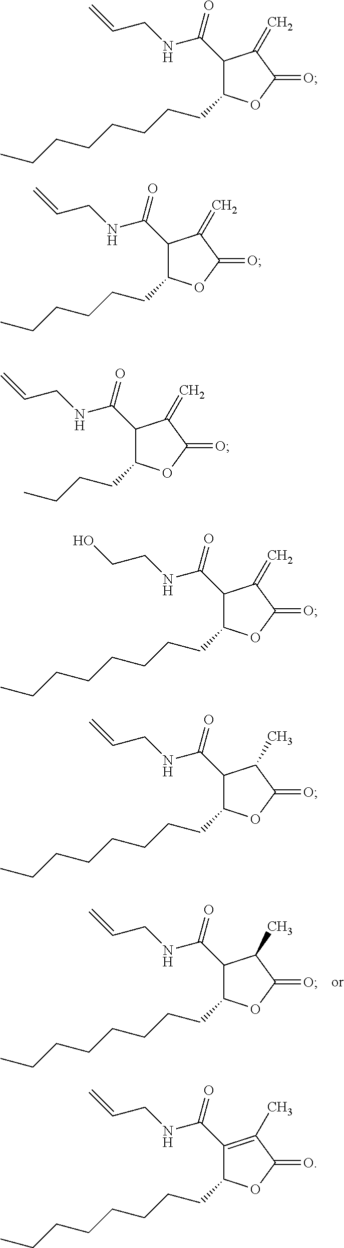

In one embodiment, the Compound of structure (XVIII) is not C75.

In one embodiment, a the modulator of a host cell target is a compound with the following structure (XIX):

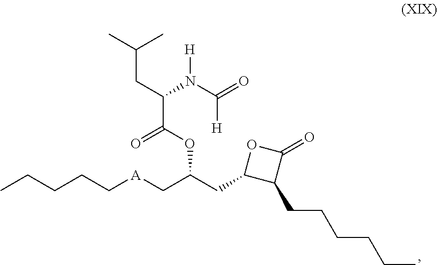

where A is —(CH₂)_{X}— or

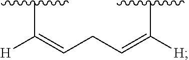

and where x is from 0 to 6.

Compounds of structure (XIX) can be made using organic synthesis techniques known to those skilled in the art, as well as by the methods described by Hadvary et. al. (U.S. Pat. No. 4,958,089), which is incorporated herein by reference in its entirety (particularly at column 8, line 1 to page 11, line 10). Further, specific examples of these compounds can be found in this publication.

A specific example of a compound of structure (XIX) is:

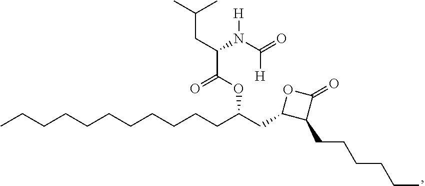 [SMILES: CCCCCCCCCCC[C@@H](C[C@@H]1OC(=O)[C@H]1CCCCCC)OC(=O)[C@H](CC(C)C)NC=O]

which is also identified as orlistat.

In another embodiment a Compound of structure (XIX) is:

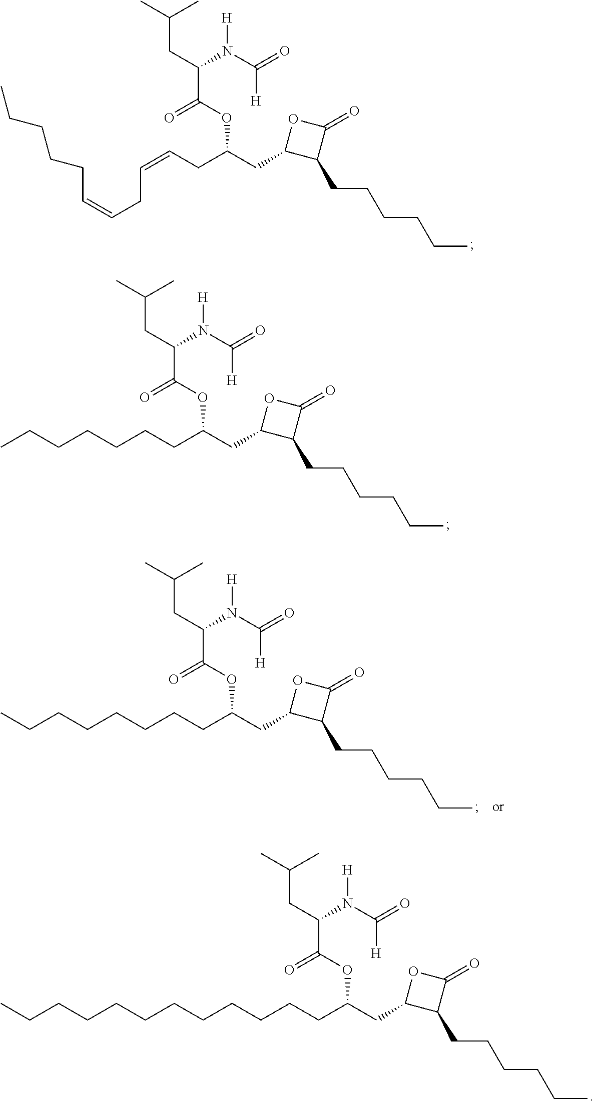

In one embodiment, the compound of structure (XIX) is not Orlistat.

In one embodiment, a the modulator of a host cell target is a compound that inhibits FAS with the following structure (XX):

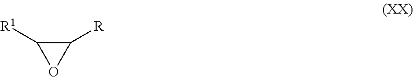

wherein:
R is selected from —CH₂OH, —CO₂R², —CONR³R⁴ or COR⁵, wherein R² is hydrogen or a lower alkyl group, R³ and R⁴ are each independently hydrogen or a lower alkyl group, R⁵ is an amino acid residue bound via a terminal nitrogen on said amino acid or a peptide having at least two amino acid residues; and
wherein R¹ is aralkyl, aralkyl(lower alkyl)ether or C₅-C₁₃ alkyl(lower alkyl)ether.

Compounds of structure (XX) can be made using organic synthesis techniques known to those skilled in the art, as well as by the methods described in U.S. Pat. No. 6,153,589, issued Nov. 28, 2000, which is incorporated herein by reference in its entirety (particularly at column 4, line 21 to column 17, line 24). Further, specific examples of these compounds can be found in this patent.

In one embodiment, the compounds of structure (XX) do not have activity against a retrovirus.

In another embodiment, the compounds of structure (XX) do not have activity against a virus which encodes for a protease.

In another embodiment, the compounds of structure (XX) do not have activity against Type C retroviruses, Type D retroviruses, HTLV-1, HTLV-2, HIV-1, HIV-2, murine leukemia virus, murine mammary tumor virus, feline leukemia virus, bovine leukemia virus, equine infectious anemia virus, or avian sarcoma viruses such as rous sarcoma virus.

In another embodiment, the compound of structure (XX) is: 2R-cis-Nonyloxirane methanol, 2S-cis-Nonyloxirane methanol, 2R-cis-Heptyloxirane methanol, 2S-cis-Heptyloxirane methanol, 2R-cis-(Heptyloxymethyl) oxirane, methanol, 2S-cis-(Heptyloxymethyl) oxirane, methanol, 2-cis-Undecyloxirane methanol, 2R-cis-(Bcnzyloxymethyl) oxirane, methanol, 2S-cis-(Bcnzyloxymethyl) oxirane methanol, cis-2-Epoxydecene, 2R-trans-Nonyloxirane methanol, 2S-trans-Nonyloxirane methanol, 2R-trans-Heptyloxirane methanol, 2S-trans-Heptyloxirane methanol, 2R-trans-Undecyloxirane methanol, 2S-trans-Undecyloxirane methanol, 2-trans-Undecyloxirane methanol, 2R-cis-Nonyloxiranecarboxylic acid, 2S-cis-Nonyloxiranecarboxylic acid, 2R-cis-Heptyloxiranecarboxylic acid, 2S-cis-Heptyloxiranecarboxylic acid, 2-cis-Undecyloxiranecarboxylic acid, 2R-trans-Nonyloxiranecarboxylic acid, 2S-trans-Nonyloxiranecarboxylic acid, 2R-trans-Undecyloxiranecarboxylic acid, 2S-trans-Undecyloxirane carboxylic acid, 2R-cis-Nonyloxiranecarboxy amide, 2S-cis-Nonyloxiranecarboxy amide, N,N-Diethyl-2R-Cis-nonloxiranecarboxy amide, or N-(2R-cis-Nonyloxiraneacyl)-L-proline methyl ester.

In one embodiment, a the modulator of a host cell target is a compound that inhibits FAS with the following structure (XXI):

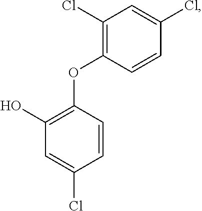 [SMILES: Oc1cc(Cl)ccc1Oc1ccc(Cl)cc1Cl]

which is also referred to as triclosan.

In one embodiment, a the modulator of a host cell target is a compound that inhibits FAS with the following structure (XXII):

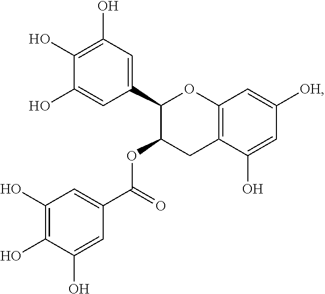 [SMILES: O=C(O[C@@H]1Cc2c(O)cc(O)cc2O[C@@H]1c1cc(O)c(O)c(O)c1)c1cc(O)c(O)c(O)c1]

which is also referred to as epigallocatechin-3-gallate.

In one embodiment, a the modulator of a host cell target is a naturally occurring flavonoid. In a particular embodiment, a compound is one of the following naturally occurring flavonoids:

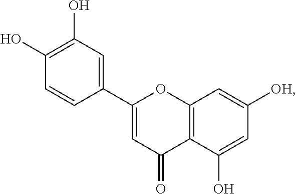 [SMILES: O=c1cc(-c2ccc(O)c(O)c2)oc2cc(O)cc(O)c12]

which is also referred to as luteolin;

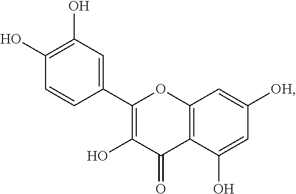 [SMILES: O=c1c(O)c(-c2ccc(O)c(O)c2)oc2cc(O)cc(O)c12]

which is also referred to as quercetin; or

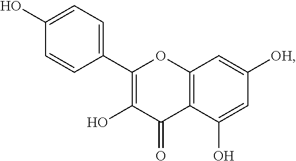 [SMILES: O=c1c(O)c(-c2ccc(O)cc2)oc2cc(O)cc(O)c12]

which is also referred to as kaempferol.

In one embodiment, the compound is CBM-301106.

1.2.9 HMG-CoA Reductase Inhibitors

In particular embodiments, the modulator of a host cell target is a HMG-CoA reductase inhibitor. Exemplary HMG-CoA reductase inhibitors are well known in the art and include, but are not limited to, mevastatin and related molecules (e.g., see U.S. Pat. No. 3,983,140); lovastatin (mevinolin) and related molecules (e.g., see U.S. Pat. No. 4,231,938); fluvastatin and related molecules; pravastatin and related molecules (e.g., see U.S. Pat. No. 4,346,227); simvastatin and related molecules (e.g., see U.S. Pat. Nos. 4,448,784 and 4,450,171); fluvastatin (e.g., see U.S. Pat. No. 5,354,772); cerivastatin see U.S. Pat. Nos. 5,006,530 and 5,177,080); atorvastatin (e.g., see U.S. Pat. Nos. 4,681,893, 5,273,995, 5,385,929 and 5,686,104); itavastatin (e.g., see U.S. Pat. No. 5,011,930); Shionogi-Astra/Zeneca visastatin (ZD-4522) (e.g., see U.S. Pat. No. 5,260,440), related statin compounds (e.g., see U.S. Pat. No. 5,753,675); pyrazole analogs of mevalonolactone derivatives (e.g., see U.S. Pat. No. 4,613,610); indene analogs of mevalonolactone derivatives (e.g., see International Patent Application Publication No. WO 1986/03488); 6-[2-(substituted-pyrrol-1-yl)-alkyl)pyran-2-ones and derivatives thereof (e.g., see U.S. Pat. No. 4,647,576); Searle's SC-45355 (a 3-substituted pentanedioic acid derivative) dichloroacetate, imidazole analogs of mevalonolactone (e.g., see International Patent Application No. WO 1986/07054); 3-carboxy-2-hydroxy-propane-phosphonic acid derivatives; naphthyl analogs of mevalonolactone (e.g., see U.S. Pat. No. 4,686,237); octahydronaphthalenes (e.g., see U.S. Pat. No. 4,499,289); keto analogs of mevinolin (lovastatin); phosphinic acid compounds (e.g., see GB 2205837); and quinoline and pyridine derivatives (e.g., see U.S. Pat. Nos. 5,506,219 and 5,691,322). Each of the references above is incorporated by reference herein in its entirety. The structures of such exemplary HMG-CoA reductase inhibitors are well known in the art.

1.2.10 Inhibitor of Serine Palmitoyl Transferase (SPT)

In one embodiment, the modulator of a host cell target is a compound that is an inhibitor of serine palmitoyl transferase (SPT) or a prodrug thereof, or pharmaceutically acceptable salt or ester of said compound or prodrug. In one embodiment the inhibitor of SPT is myriocin, sphingofungin B, sphingofungin C, sphingofungin E sphingofungin F, lipoxamycin, viridiofungin A, sulfamisterin, or NA255

### 2. Modulators of HCV-Associated Components

According to the present invention, the antiviral combination therapy includes the administration of (i) one or more modulators of the host cell targets described herein, and (ii) one or more modulator of an HCV-associated component. Combinations of the modulators of an HCV-associated component that may be administered as part of a combination therapy along with a modulator of the host cell target includes, for example, an HCV protease inhibitor and an HCV helicase (NS3) inhibitor, or other combinations of modulators of an HCV-associated component where the modulators effect different HCV targets. In one embodiment the combination therapy includes the administration of one or more modulators of a host cell target and two or more modulators of an HCV-associated component were the modulators of an HCV-associated component effect the same HCV target.

Compounds that modulate the activity of an HCV-associated component inhibit or prevent viral entry, integration, growth and/or production by directly effecting the function of viral proteins or by effecting the function of host cell proteins or nucleic acids that directly interact with viral proteins. The antiviral compounds disclosed herein are available, commercially or otherwise, from sources known to those skilled in the art. The compounds that modulate the activity of an HCV-associated component are distinguished from the modulators of host cell targets described herein in that the modulators of host cell targets do not directly effect the function of viral proteins or host cell proteins and nucleic acids that directly interact with viral proteins.

2.1 Ribavirin and Analogues

Ribavirin is a nucleoside analogue that is used to treat infections by a variety DNA and RNA viruses. Analogues of ribavirin include taribavirin, mizoribine, viramidine, merimepodib, mycophenolate mofetil, and mycophenolate.

2.2 HCV Protease Inhibitors

HCV has a 9.6-kb plus-strand RNA genome that encodes a polyprotein precursor of about 3,000 amino acids. This polyprotein precursor is cleaved by both cellular and viral proteases to 10 individual proteins, including four structural proteins (C, E1, E2, and p′7) and six nonstructural proteins (NS2, NS3, NS4A, NS4B, NS5A, and NS5B). NS2 and the protease domain of NS3 (from aa 810 to 1206) constitute NS2/3, which undergoes autocatalytic cleavage between aa 1026 and 1027 (the NS2/NS3 boundary). NS3 consists of an N-terminal serine protease domain and a C-terminal helicase domain. NS3 forms a noncovalent complex with the NS4A, and cleaves the polyprotein precursor at four locations: NS3/4A (self cleavage), NS4A/4B, NS4B/5A, and NS5A/5B.

The NS3/4A serine protease also contributes to the ability of HCV to evade early innate immune responses. NS3/4A has been shown to block virus induced activation of IFN regulatory factor 3 (IRF-3), a transcription factor playing a critical role in the induction of type-1 IFNs.

In one embodiment, the invention provides for treatment or amelioration of HCV infection and replication comprising administering a combination therapy that includes an agent that modulates a cellular target and an HCV protease inhibitor. HCV protease inhibitors include, without limitation,

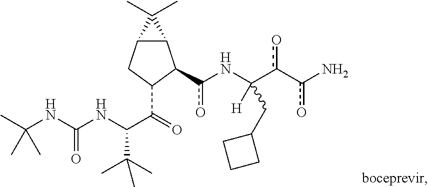

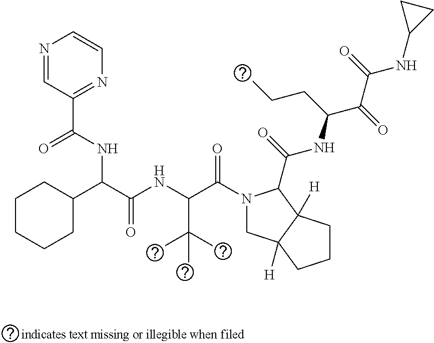 [SMILES: CCC[C@H](NC(=O)C1C2CCCC2CN1C(=O)C(NC(=O)C(NC(=O)c1cnccn1)C1CCCCC1)C(C)(C)C)C(=O)C(=O)NC1CC1]

telaprevir (VX-950), ITMN-191, SCH-900518, TMC-435, BI-201335, MK-7009, VX-500, VX-813, BMS650032, VBY376, R7227, VX-985, ABT-333, ACH-1625, ACH-2684, GS-9256 GS-9451, MK-5172 and ABT-450.

2.3 Helicase (NS3) inhibitors

In one embodiment, the invention provides for treatment or amelioration of HCV infection and replication comprising a combination therapy that includes an agent that inhibits a cellular target and an HCV helicase (NS3) inhibitor. HCV helicase inhibitors include, but are not limited to compounds of the following structure:

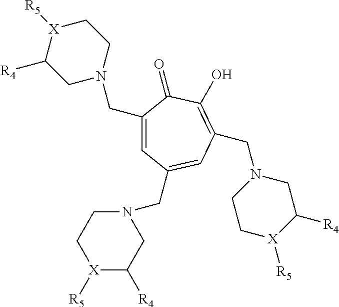

wherein X is N, R₄ is H and R₅ is CH₃: X is CH, R₄ is H and R₅ is CH₃; or X is CH, R₄ is CH₃ and R₅ is H (see Najda-Bernatowicza et al., 2010, Bioorg. & Med. Chem. 18(14):5129-5136).

Additional NS3 helicase inhibitors include compounds disclosed by Gemma et al. (Bioorg. Med. Chem. Lett. (2011) 21(9):2776-2779), which is incorporated herein by reference (see particularly, table 1). Such compounds include:

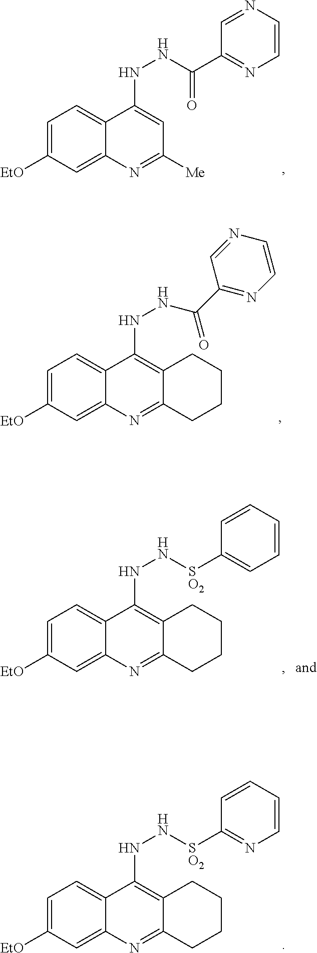 [SMILES: CCOc1ccc2c(NNC(=O)c3cnccn3)cc(C)nc2c1 ; CCOc1ccc2c(NNC(=O)c3cnccn3)c3c(nc2c1)CCCC3 ; CCOc1ccc2c(NNS(=O)(=O)c3ccccc3)c3c(nc2c1)CCCC3 ; CCOc1ccc2c(NNS(=O)(=O)c3ccccn3)c3c(nc2c1)CCCC3]

Another NS3 inhibitor is

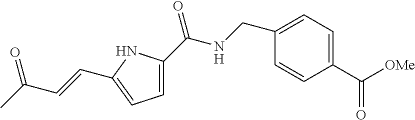 [SMILES: COC(=O)c1ccc(CNC(=O)c2ccc(/C=C/C(C)=O)[nH]2)cc1]

(see, Kandil et al., 2009, Bioorg. Med. Chem. Lett. 19(11), 2935-7).

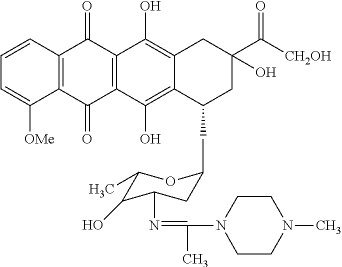 [SMILES: COc1cccc2c1C(=O)c1c(O)c3c(c(O)c1C2=O)CC(O)(C(=O)CO)C[C@@H]3CC1CC(/N=C(/C)N2CCN(C)CC2)C(O)C(C)O1]

Another NS3 inhibitor is (see Krawczyk et al., 2009, Biol Chem. 390(4), 351-60). Another NS3 inhibitor is

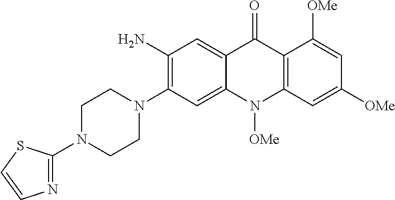 [SMILES: COc1cc(OC)c2c(=O)c3cc(N)c(N4CCN(c5nccs5)CC4)cc3n(OC)c2c1]

(see Manfroni et al., 2009, J. Med. Chem. 52(10), 3354-65).

Other NS3 inhibitors include

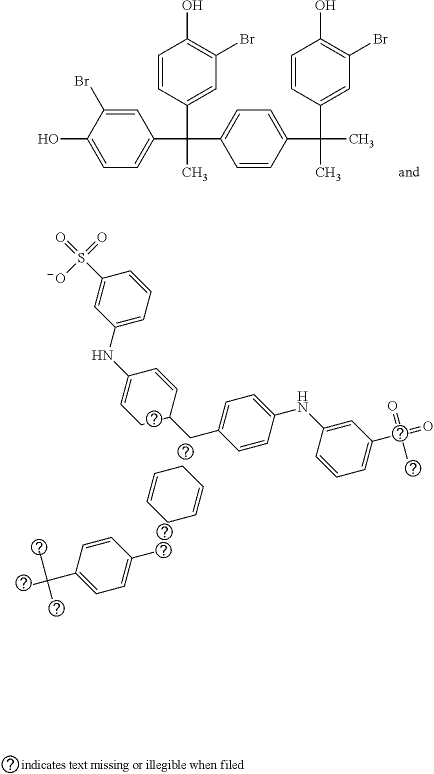

(Soluble Blue HT) (see Chen et al., 2009, J. Med. Chem. 52, 2716-23).

In general, it is preferable for HCV helicase inhibitors to be selective for NS3 so that there is an effective inhibitory concentration that has little or no cytoxicity. Nonetheless, when administered with an agent that modulates a cellular target, the amount of the NS3 inhibitor that is used can be reduced to minimize cytoxicity.

2.4 Nonstructural Protein (NS4B, Membrane Alterations) Inhibitors

NS4B is a 27-kDa membrane protein that is primarily involved in the formation of membrane vesicles—also named membranous web-used as scaffold for the assembly of the HCV replication complex. In addition, NS4B contains NTPase and RNA binding activities, as well as anti-apoptotic properties.

In one embodiment, the invention provides for treatment or amelioration of HCV infection and replication comprising a combination therapy that includes an agent that modulates a cellular target and an HCV nonstructural protein 4B (NS4B) inhibitor. Inhibitors of the HCV NS4B protein include, but are not limited to, GSK-8853, clemizole, and other NS4B-RNA binding inhibitors, including but not limited to benzimidazole RBIs (B-RBIs) and indazole RBIs (I-RBIs).

2.5 Nonstructural Protein (NSSA, Phosphoprotein) Inhibitors

In one embodiment, the invention provides for treatment or amelioration of HCV infection and replication comprising a combination therapy that includes an agent that modulates a cellular target and an HCV nonstructural protein 5A (NSSA) inhibitor. HCV NSSA inhibitors include, but are not limited to, BMS-790052, A-689, A-831, EDP239, GS5885, GSK805, PPI-461 BMS-824393 and ABT-267.

2.6 Polymerase (NS5B) Inhibitors

In one embodiment, the invention provides for treatment or amelioration of HCV infection and replication comprising administering a combination therapy that includes an agent that modulates a cellular target and an HCV polymerase (NS5B) inhibitor. HCV polymerase inhibitors include, but are not limited to nucleoside analogs (e.g., valopicitabine, R1479, R1626, R7128, RG7128 (mericitabine, an ester prodrug of PSI-6130), TMC649128), nucleotide analogs (e.g., IDX184, PSI-352938 (PSI-938), INX-08189 (INX-189), GS6620), and non-nucleoside analogs (e.g., filibuvir, HCV-796, VCH-759, VCH-916, ANA598, VCH-222 (VX-222), BI-207127, MK-3281, ABT-072, ABT-333, GS9190, BMS791325, GSK2485852A).

In some embodiments, the direct-acting antiviral within the scope of the present invention is the HCV NS5B polymerase inhibitor PSI-7851, which is a mixture of the two diastereomers PSI-7976 and PSI-7977. See Sofia et al., J. Med. Chem., 2010, 53:7202-7218; see also Murakami et al, J. Biol. Chem., 2010, 285:34337-34347. In other embodiments, the direct-acting antiviral within the scope of the present invention is PSI-7976 or PSI-7977. PSI-7851 has the structural formula depicted in the formula below:

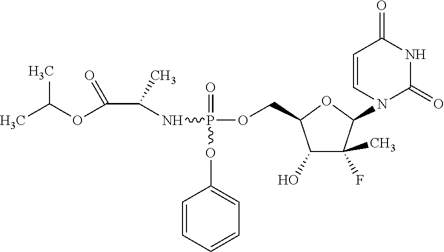

The molecular formula of PSI-7851 is C₂₂H₂₉FN₃O₉P and its molecular weight is 529.45 g/mol. Compound PSI-7976 has the structural formula depicted in the formula below:

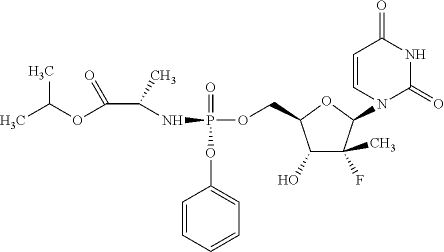 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

Compound PSI-7977 has the structural formula depicted in the formula below:

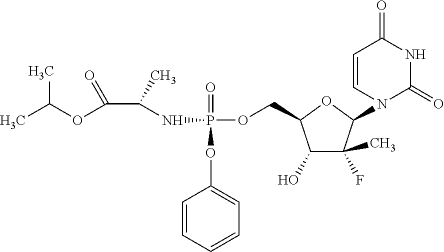 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

The CAS Registry Number of PSI-7977 is 1190307-88-0. Both racemic and non-racemic mixtures of compounds PSI-7976 and PSI-7977 are within the scope of the present invention.

2.7 Viral Ion Channel Forming Protein (p7) Inhibitors

In one embodiment, the invention provides for treatment or amelioration of HCV infection and replication comprising administering a combination therapy that includes an agent that inhibits a cellular target and an inhibitor of HCV viral ion channel forming protein (P7). HCV P7 inhibitors include, without limitation, BIT225 and HPH116.

2.8 HCV RNAi

In one embodiment, the invention provides for treatment or amelioration of HCV infection and replication comprising administering a combination therapy that includes an agent that modulates a cellular target and an HCV RNAi. Such inhibitory polynucleotides include, but are not limited to, TT033, TT034, Sirna-AV34, and OBP701.

2.9 Internal Ribosome Entry Site (IRES) Inhibitors

Other direct acting antiviral agents are IRES inhibitors, which include Mifepristone, Hepazyme, ISIS14803, and siRNAs/shRNAs.

2.10 HCV Entry Inhibitors

Other direct acting antiviral agents are HCV entry inhibitors, which include HuMax HepC (an E2-antibody), JTK-652, PRO206, SP-30, and ITX5061.

2.11 Cyclophilin Inhibitors

Cyclophilins (e.g., cyclophilin B, also known as peptidylprolyl isomerase B) are host enzymes that regulate viral targets. Cyclophilin B regulates HCV RNA polymerase (NS5B). With respect to HCV, compounds that bind to NS5B and inhibit binding of cycolphilin B are referred to as cyclophilin inhibitors. In one embodiment, the invention provides for treatment or amelioration of HCV infection and replication comprising administering a combination therapy that includes an agent that inhibits a cellular target and a cyclophilin inhibitor, for example Debio 025 (alisporivir), NIM811, SCY-635, and cyclosporin-A.

2.12 MicroRNA Antagonists

MicroRNA-122 (miR-122) is thought to stimulate HCV replication through interaction with the HCV 5′ untranslated region. In one embodiment, a modulator of a host cell target is a administered as part of a combination therapy that includes an agent that inhibits microRNA-122 (miR-122). SPC3649 (miravirsen) is a locked nucleic acid (LNA)-modified oligonucleotide complementary to miR-122.

3. Other Agents that Act at Least Partly on a Host Factor

3.1 Immunomodulators

According to the invention, a modulator of a host cell target is administered as part of a combination therapy that includes an immunomodulator effective to reduce or inhibit HCV. Immunomodulators include several types of compounds. Non-limiting examples include inteferons (e.g., Pegasys, Roferon-A, Pegintron, Intron A, Albumin IFN-α, locteron, Peginterferon-λ, omega-IFN, medusa-IFN, belerofon, infradure, Interferon alfacon-1, and Veldona), caspase/pan-caspase inhibitors (e.g., emricasan, nivocasan, IDN-6556, GS9450), Toll-like receptor agonists (e.g., Actilon, ANA773, IMO-2125, SD-101), cytokines and cytokine agonists and antagonists (e.g., ActoKine-2, Interleukin 29, Infliximab (cytokine TNFα blocker), IPH1101 (cytokine agonist), and other immunomodulators such as, without limitation, thymalfasin, Eltrombopag, IP1101, SCV-07, Oglufanide disodium, CYT107, ME3738, TCM-700C, EMZ702, EGS21.

3.2 Inhibitors of Microtubules

In one embodiment a modulator of a host cell target is administered as part of a combination therapy that includes an inhibitor of microtubule polymerization. Non-limiting examples of microtubule polymerization inhibitors include colchicine, Prazosin, and mitoquinone. Farglitazar and GI262570 are PPAR-gamma inhibitors that reduce tubulin levels without affecting the polymerization of tubulin. These compounds target tubulin itself, rather than the equilibrium between tubulin and microtubules.

3.3 Host Metabolism Inhibitors

In another such embodiment, a modulator of a host cell target is as part of a combination therapy that includes a host metabolism inhibitor. Examples of host metabolism inhibitors include Hepaconda (bile acid and cholesterol secretion inhibitor), Miglustat (glucosylceramide synthase inhibitor), Celgosivir (alpha glucosidase inhibitor), Methylene blue (Monoamine oxidase inhibitor), pioglitazone and metformin (insulin regulator), Nitazoxanide (possibly PFOR inhibitor), NA255 and NA808 (Serine palmitoyltransferase inhibitor), NOV205 (Glutathione-S-transferase activator), and ADIPEG20 (arginine deiminase).

In another such embodiment, a modulator of a host cell target part of a combination therapy that includes an agent selected from laccase (herbal medicine), silibinin and silymarin (antioxidant, hepato-protective agent), PYN17 and JKB-122 (anti-inflammatory), CTS-1027 (matrix metalloproteinase inhibitor), Lenocta (protein tyrosine phosphatase inhibitor), Bavituximab and BMS936558 (programmed cell death inhibitor), HepaCide-I (nano-viricide), CF102 (Adenosine A3 receptor), GNS278 (inhibits viral-host protein interaction by attacking autophagy), RPIMN (Nicotinic receptor antagonist), PYN18 (possible viral maturation inhibitor), ursa and Hepaconda (bile acids, possible farnesoid X receptor), tamoxifen (anti-estrogen), Sorafenib (kinase inhibitor), KPE02001003 (unknown mechanism).

### 4. Screening Assays to Identify Inhibitors of Host Cell Target Enzymes

Compounds known to be inhibitors of the host cell target enzymes can be directly screened for antiviral activity using assays known in the art and/or described infra (see, e.g., Section 5 et seq.). While optional, derivatives or congeners of such enzyme inhibitors, or any other compound can be tested for their ability to modulate the enzyme targets using assays known to those of ordinary skill in the art and/or described below. Compounds found to modulate these targets can be further tested for antiviral activity. Compounds found to modulate these targets or to have antiviral activity (or both) can also be tested in the metabolic flux assays described in Section 5.2.8 in order to confirm the compound's effect on the metabolic flux of the cell. This is particularly useful for determining the effect of the compound in blocking the ability of the virus to alter cellular metabolic flux, and to identify other possible metabolic pathways that may be targeted by the compound.

Alternatively, compounds can be tested directly for antiviral activity. Those compounds which demonstrate anti-viral activity, or that are known to be antiviral but have unacceptable specificity or toxicity, can be screened against the enzyme targets of the invention. Antiviral compounds that modulate the enzyme targets can be optimized for better activity profiles.

Any host cell enzyme, known in the art and/or described in Section 5.1, is contemplated as a potential target for antiviral intervention. Further, additional host cell enzymes that have a role, directly or indirectly, in regulating the cell's metabolism are contemplated as potential targets for antiviral intervention. Compounds, such as the compounds disclosed herein or any other compounds, e.g., a publicly available library of compounds, can be tested for their ability to modulate (activate or inhibit) the activity of these host cell enzymes. If a compound is found to modulate the activity of a particular enzyme, then a potential antiviral compound has been identified.

In one embodiment, an enzyme that affects or is involved in synthesis of long and very long chain fatty acids is tested as a target for the compound, for example, ACSL1, ELOVL2, ELOVL3, ELOVL6, or SLC27A3. In one embodiment, long and very long chain acyl-CoA synthases are tested for modulation by the compound. In another embodiment, fatty acid elongases are tested for modulation by the compound. In one embodiment, an enzyme involved in synthesis of cysteinyl leukotrienes is tested for modulation by the compound. In one embodiment, an enzyme that plays role in lipid storage (including but not limited to ADP-ribosyltransferase 1 or 3) is tested for modulation by the compound. In another embodiment, an alanine-glyoxylate aminotransferase is tested for modulation by the compound. In yet another embodiment, an enzyme in the pentose phosposphate pathway is is tested for modulation by the compound.

In preferred embodiments, a compound is tested for its ability to modulate host metabolic enzymes by contacting a composition comprising the compound with a composition comprising the enzyme and measuring the enzyme's activity. If the enzyme's activity is altered in the presence of the compound compared to a control, then the compound modulates the enzyme's activity. In some embodiments of the invention, the compound increases an enzyme's activity (for example, an enzyme that is a negative regulator of fatty acid biosynthesis might have its activity increased by a potential antiviral compound). In specific embodiments, the compound increases an enzyme's activity by at least approximately 10%, 15%, 20%, 25%, 30%, 40%, 50%, 60%, 70%, 80% or 90%. In some embodiments, the compound decreases an enzyme's activity. In particular embodiments, the compound decreases an enzyme's activity by at least approximately 10%, 15%, 20%, 25%, 30%, 40%, 50%, 60%, 70%, 80%, 90%, 95% or 100%. In certain embodiments, the compound exclusively modulates a single enzyme. In some embodiments, the compound modulates multiple enzymes, although it might modulate one enzyme to a greater extent than another. Using the standard enzyme activity assays described herein, the activity of the compounds could be characterized. In one embodiment, a compound exhibits an irreversible inhibition or activation of a particular enzyme. In some embodiments, a compound reversibly inhibits or activates an enzyme. In some embodiments, a compound alters the kinetics of the enzyme.

In one embodiment, for example, evaluating the interaction between the test compound and host target enzyme includes one or more of (i) evaluating binding of the test compound to the enzyme; (ii) evaluating a biological activity of the enzyme; (iii) evaluating an enzymatic activity (e.g., elongase activity) of the enzyme in the presence and absence of test compound. The in vitro contacting can include forming a reaction mixture that includes the test compound, enzyme, any required cofactor (e.g., biotin) or energy source (e.g., ATP, or radiolabeled ATP), a substrate (e.g., acetyl-CoA, a sugar, a polypeptide, a nucleoside, or any other metabolite, with or without label) and evaluating conversion of the substrate into a product. Evaluating product formation can include, for example, detecting the transfer of carbons or phosphate (e.g., chemically or using a label, e.g., a radiolabel), detecting the reaction product, detecting a secondary reaction dependent on the first reaction, or detecting a physical property of the substrate, e.g., a change in molecular weight, charge, or pI.

Target enzymes for use in screening assays can be purified from a natural source, e.g., cells, tissues or organs comprising adipocytes (e.g., adipose tissue), liver, etc. Alternatively, target enzymes can be expressed in any of a number of different recombinant DNA expression systems and can be obtained in large amounts and tested for biological activity. For expression in recombinant bacterial cells, for example E. coli, cells are grown in any of a number of suitable media, for example LB, and the expression of the recombinant polypeptide induced by adding IPTG to the media or switching incubation to a higher temperature. After culturing the bacteria for a further period of between 2 and 24 hours, the cells are collected by centrifugation and washed to remove residual media. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars such as sucrose into the buffer and centrifugation at a selective speed. If the recombinant polypeptide is expressed in the inclusion, these can be washed in any of several solutions to remove some of the contaminating host proteins, then solubilized in solutions containing high concentrations of urea (e.g., 8 M) or chaotropic agents such as guanidine hydrochloride in the presence of reducing agents such as beta-mercaptoethanol or DTT (dithiothreitol). At this stage it may be advantageous to incubate the polypeptide for several hours under conditions suitable for the polypeptide to undergo a refolding process into a conformation which more closely resembles that of the native polypeptide. Such conditions generally include low polypeptide (concentrations less than 500 mg/ml), low levels of reducing agent, concentrations of urea less than 2 M and often the presence of reagents such as a mixture of reduced and oxidized glutathione which facilitate the interchange of disulphide bonds within the protein molecule. The refolding process can be monitored, for example, by SDS-PAGE or with antibodies which are specific for the native molecule. Following refolding, the polypeptide can then be purified further and separated from the refolding mixture by chromatography on any of several supports including ion exchange resins, gel permeation resins or on a variety of affinity columns.

Isolation and purification of host cell expressed polypeptide, or fragments thereof may be carried out by conventional means including, but not limited to, preparative chromatography and immunological separations involving monoclonal or polyclonal antibodies.

These polypeptides may be produced in a variety of ways, including via recombinant DNA techniques, to enable large scale production of pure, biologically active target enzyme useful for screening compounds for the purposes of the invention. Alternatively, the target enzyme to be screened could be partially purified or tested in a cellular lysate or other solution or mixture.

Target enzyme activity assays are preferably in vitro assays using the enzymes in solution or using cell or cell lysates that express such enzymes, but the invention is not to be so limited. In certain embodiments, the enzyme is in solution. In other embodiments, the enzyme is associated with microsomes or in detergent. In other embodiments, the enzyme is immobilized to a solid or gel support. In certain embodiments, the enzyme is labeled to facilitate purification and/or detection. In other embodiments, a substrate is labeled to facilitate purification and or detection. Labels include polypeptide tags, biotin, radiolabels, fluorescent labels, or a colorimetric label. Any art-accepted assay to test the activity of metabolic enzymes can be used in the practice of this invention. Preferably, many compounds are screened against multiple targets with high throughput screening assays.

Substrate and product levels can be evaluated in an in vitro system, e.g., in a biochemical extract, e.g., of proteins. For example, the extract may include all soluble proteins or a subset of proteins (e.g., a 70% or 50% ammonium sulfate cut), the useful subset of proteins defined as the subset that includes the target enzyme. The effect of a test compound can be evaluated, for example, by measuring substrate and product levels at the beginning of a time course, and then comparing such levels after a predetermined time (e.g., 0.5, 1, or 2 hours) in a reaction that includes the test compound and in a parallel control reaction that does not include the test compound. This is one method for determining the effect of a test compound on the substrate-to-product ratio in vitro. Reaction rates can obtained by linear regression analysis of radioactivity or other label incorporated vs. reaction time for each incubation. K_{M} and Vₘₐₓ values can be determined by non-linear regression analysis of initial velocities, according to the standard Henri-Michaelis-Menten equation. k_{cat} can be obtained by dividing Vₘₐₓ values by reaction concentrations of enzyme, e.g., derived by colorimetric protein determinations (e.g., Bio-RAD protein assay, Bradford assay, Lowry method). In one embodiment, the compound irreversibly inactivates the target enzyme. In another embodiment, the compound reversibly inhibits the target enzyme. In some embodiments, the compound reversibly inhibits the target enzyme by competitive inhibition. In some embodiments, the compound reversibly inhibits the target enzyme by noncompetitive inhibition. In some embodiments, the compound reversibly inhibits the target enzyme by uncompetitive inhibition. In a further embodiment, the compound inhibits the target enzyme by mixed inhibition. The mechanism of inhibition by the compound can be determined by standard assays known by those of ordinary skill in the art.

Methods for the quantitative measurement of enzyme activity utilizing a phase partition system are described in U.S. Pat. No. 6,994,956, which is incorporated by reference herein in its entirety. Specifically, a radiolabeled substrate and the product of the reaction are differentially partitioned into an aqueous phase and an immiscible scintillation fluid-containing organic phase, and enzyme activity is assessed either by incorporation of a radiolabeled-containing organic-soluble moiety into product molecules (gain of signal assay) or loss of a radiolabel-containing organic-soluble moiety from substrate molecules (loss of signal assay). Scintillations are only detected when the radionuclide is in the organic, scintillant-containing phase. Such methods can be employed to test the ability of a compound to inhibit the activity of a target enzyme.

Cellular assays may be employed. An exemplary cellular assay includes contacting a test compound to a culture cell (e.g., a mammalian culture cell, e.g., a human culture cell) and then evaluating substrate and product levels in the cell, e.g., using any method described herein, such as Reverse Phase HPLC, LC-MS, or LC-MS/MS.

Substrate and product levels can be evaluated, e.g., by NMR, HPLC (See, e.g., Bak, M. I., and Ingwall, J. S. (1994) J. Clin. Invest. 93, 40-49), mass spectrometry, thin layer chromatography, or the use of radiolabeled components (e.g., radiolabeled ATP for a kinase assay). For example, ³¹P NMR can be used to evaluate ATP and AMP levels. In one implementation, cells and/or tissue can be placed in a 10-mm NMR sample tube and inserted into a 1H/31P double-tuned probe situated in a 9.4-Tesla superconducting magnet with a bore of 89 cm. If desired, cells can be contacted with a substance that provides a distinctive peak in order to index the scans. Six ³¹P NMR spectra—each obtained by signal averaging of 104 free induction decays—can be collected using a 60° flip angle, 15-microsecond pulse, 2.14-second delay, 6,000 Hz sweep width, and 2048 data points using a GE-400 Omega NMR spectrometer (Bruker Instruments, Freemont, Calif., USA). Spectra are analyzed using 20-Hz exponential multiplication and zero- and first-order phase corrections. The resonance peak areas can be fitted by Lorentzian line shapes using NMR1 software (New Methods Research Inc., Syracuse, N.Y., USA). By comparing the peak areas of fully relaxed spectra (recycle time: 15 seconds) and partially saturated spectra (recycle time: 2.14 seconds), the correction factor for saturation can be calculated for the peaks. Peak areas can be normalized to cell and/or tissue weight or number and expressed in arbitrary area units. Another method for evaluating, e.g., ATP and AMP levels includes lysing cells in a sample to form an extract, and separating the extract by Reversed Phase HPLC, while monitoring absorbance at 260 nm.

Another type of in vitro assay evaluates the ability of a test compound to modulate interaction between a first enzyme pathway component and a second enzyme pathway component. This type of assay can be accomplished, for example, by coupling one of the components with a radioisotope or enzymatic label such that binding of the labeled component to the second pathway component can be determined by detecting the labeled compound in a complex. An enzyme pathway component can be labeled with ¹²⁵I, ³⁵S, ¹⁴C, or ³H, either directly or indirectly, and the radioisotope detected by direct counting of radio-emission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

Soluble and/or membrane-bound forms of isolated proteins (e.g., enzyme pathway components and their receptors or biologically active portions thereof) can be used in the cell-free assays of the invention. When membrane-bound forms of the enzyme are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate. In another example, the enzyme pathway component can reside in a membrane, e.g., a liposome or other vesicle.

Cell-free assays involve preparing a reaction mixture of the target enzyme and the test compound under conditions and for a time sufficient to allow the two components to interact and bind, thus forming a complex that can be removed and/or detected. In one embodiment, the target enzyme is mixed with a solution containing one or more, and often many hundreds or thousands, of test compounds. The target enzyme, including any bound test compounds, is then isolated from unbound (i.e., free) test compounds, e.g., by size exclusion chromatography or affinity chromatography. The test compound(s) bound to the target can then be separated from the target enzyme, e.g., by denaturing the enzyme in organic solvent, and the compounds identified by appropriate analytical approaches, e.g., LC-MS/MS.

The interaction between two molecules, e.g., target enzyme and test compound, can also be detected, e.g., using a fluorescence assay in which at least one molecule is fluorescently labeled, e.g., to evaluate an interaction between a test compound and a target enzyme. One example of such an assay includes fluorescence energy transfer (FET or FRET for fluorescence resonance energy transfer) (See, for example, Lakowicz et al., U.S. Pat. No. 5,631,169; Stavrianopoulos, et al., U.S. Pat. No. 4,868,103). A fluorophore label on the first, “donor” molecule is selected such that its emitted fluorescent energy will be absorbed by a fluorescent label on a second, “acceptor” molecule, which in turn is able to fluoresce due to the absorbed energy. Alternately, a proteinaceous “donor” molecule may simply utilize the natural fluorescent energy of tryptophan residues. Labels are chosen that emit different wavelengths of light, such that the “acceptor” molecule label may be differentiated from that of the “donor.” Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999) Comb Chem HTS 2:177-190; Jameson et al. (1995) Methods Enzymol 246:283; See Anal Biochem. 255:257 (1998). Fluorescence polarization can be monitored in multi-well plates. See, e.g., Parker et al. (2000) Journal of Biomolecular Screening 5:77-88; and Shoeman, et al. (1999) 38, 16802-16809.

In another embodiment, determining the ability of the target enzyme to bind to a target molecule can be accomplished using real-time Biomolecular Interaction Analysis (BIA) (See, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target enzyme is anchored onto a solid phase. The target enzyme/test compound complexes anchored on the solid phase can be detected at the end of the reaction, e.g., the binding reaction. For example, the target enzyme can be anchored onto a solid surface, and the test compound (which is not anchored), can be labeled, either directly or indirectly, with detectable labels discussed herein.

It may be desirable to immobilize either the target enzyme or an anti-target enzyme antibody to facilitate separation of complexed from uncomplexed forms of one or both of the proteins, as well as to accommodate automation of the assay. Binding of a test compound to target enzyme, or interaction of a target enzyme with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/target enzyme fusion proteins can be adsorbed onto glutathione sepharose beads (Sigma Chemical, St. Louis, Mo., USA) or glutathione derivatized microtiter plates, which are then combined with the test compound or the test compound and either the non-adsorbed target enzyme, and the mixture incubated under conditions conducive to complex formation (e.g., at physiological conditions for salt and pH). Following incubation, the beads or microtiter plate wells are washed to remove any unbound components, the matrix immobilized in the case of beads, and the complex determined either directly or indirectly, for example, as described above. Alternatively, the complexes can be dissociated from the matrix, and the level of target enzyme binding or activity is determined using standard techniques.

Other techniques for immobilizing either a target enzyme or a test compound on matrices include using conjugation of biotin and streptavidin. Biotinylated target enzyme or test compounds can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with a target enzyme but which do not interfere with binding of the target enzyme to the test compound and/or substrate. Such antibodies can be derivatized to the wells of the plate, and unbound target enzyme trapped in the wells by antibody conjugation. Methods for detecting such complexes, in addition to those described above for the GST-immobilized complexes, include immunodetection of complexes using antibodies reactive with the target enzyme, as well as enzyme-linked assays which rely on detecting an enzymatic activity associated with the target enzyme.

Alternatively, cell free assays can be conducted in a liquid phase. In such an assay, the reaction products are separated from unreacted components, by any of a number of standard techniques, including but not limited to: differential centrifugation (See, for example, Rivas, G., and Minton, A. P., (1993) Trends Biochem Sci 18:284-7); chromatography (gel filtration chromatography, ion-exchange chromatography); electrophoresis (See, e.g., Ausubel, F. et al., eds. Current Protocols in Molecular Biology 1999, J. Wiley: New York); and immunoprecipitation (See, for example, Ausubel, F. et al., eds. (1999) Current Protocols in Molecular Biology, J. Wiley: New York). Such resins and chromatographic techniques are known to one skilled in the art (See, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the target enzyme or biologically active portion thereof with a known compound which binds the target enzyme to form an assay mixture, contacting the assay mixture with a test compound, and determining the ability of the test compound to interact with the target enzyme, wherein determining the ability of the test compound to interact with the target enzyme includes determining the ability of the test compound to preferentially bind to the target enzyme, or to modulate the activity of the target enzyme, as compared to the known compound (e.g., a competition assay). In another embodiment, the ability of a test compound to bind to and modulate the activity of the target enzyme is compared to that of a known activator or inhibitor of such target enzyme.

The target enzymes of the invention can, in vivo, interact with one or more cellular or extracellular macromolecules, such as proteins, which are either heterologous to the host cell or endogenous to the host cell, and which may or may not be recombinantly expressed. For the purposes of this discussion, such cellular and extracellular macromolecules are referred to herein as “binding partners.” Compounds that disrupt such interactions can be useful in regulating the activity of the target enzyme. Such compounds can include, but are not limited to molecules such as antibodies, peptides, and small molecules. In an alternative embodiment, the invention provides methods for determining the ability of the test compound to modulate the activity of a target enzyme through modulation of the activity of a downstream effector of such target enzyme. For example, the activity of the effector molecule on an appropriate target can be determined, or the binding of the effector to an appropriate target can be determined, as previously described.

To identify compounds that interfere with the interaction between the target enzyme and its cellular or extracellular binding partner(s), a reaction mixture containing the target enzyme and the binding partner is prepared, under conditions and for a time sufficient, to allow the two products to form a complex. In order to test an inhibitory compound, the reaction mixture is provided in the presence and absence of the test compound. The test compound can be initially included in the reaction mixture, or can be added at a time subsequent to the addition of the target and its cellular or extracellular binding partner. Control reaction mixtures are incubated without the test compound or with a placebo. The formation of any complexes between the target product and the cellular or extracellular binding partner is then detected. The formation of a complex in the control reaction, but not in the reaction mixture containing the test compound, indicates that the compound interferes with the interaction of the target product and the interactive binding partner. Additionally, complex formation within reaction mixtures containing the test compound and normal target enzyme can also be compared to complex formation within reaction mixtures containing the test compound and mutant target enzyme. This comparison can be important in those cases wherein it is desirable to identify compounds that disrupt interactions of mutant but not normal target enzymes.

The assays described herein can be conducted in a heterogeneous or homogeneous format. Heterogeneous assays involve anchoring either the target enzyme or the binding partner, substrate, or tests compound onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target enzyme and a binding partners or substrate, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target enzyme or the interactive cellular or extracellular binding partner or substrate, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target enzyme and the interactive cellular or extracellular binding partner product or substrate is prepared in that either the target enzyme or their binding partners or substrates are labeled, but the signal generated by the label is quenched due to complex formation (See, e.g., U.S. Pat. No. 4,109,496 that utilizes this approach for immunoassays). The addition of a test substance that competes with and displaces one of the species from the preformed complex will result in the generation of a signal above background. In this way, test compounds that disrupt target enzyme-binding partner or substrate contact can be identified.

In yet another aspect, the target enzyme can be used as “bait protein” in a two-hybrid assay or three-hybrid assay (See, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent, International patent application Publication No. WO94/10300), to identify other proteins that bind to or interact with target enzyme (“target enzyme binding protein” or “target enzyme—bp”) and are involved in target enzyme pathway activity. Such target enzyme-bps can be activators or inhibitors of the target enzyme or target enzyme targets as, for example, downstream elements of the target enzyme pathway.

In another embodiment, modulators of a target enzyme's gene expression are identified. For example, a cell or cell free mixture is contacted with a candidate compound and the expression of the target enzyme mRNA or protein evaluated relative to the level of expression of target enzyme mRNA or protein in the absence of the candidate compound. When expression of the target enzyme component mRNA or protein is greater in the presence of the candidate compound than in its absence, the candidate compound is identified as a stimulator of target enzyme mRNA or protein expression. Alternatively, when expression of the target enzyme mRNA or protein is less (statistically significantly less) in the presence of the candidate compound than in its absence, the candidate compound is identified as an inhibitor of the target enzyme mRNA or protein expression. The level of the target enzyme mRNA or protein expression can be determined by methods for detecting target enzyme mRNA or protein, e.g., Westerns, Northerns, PCR, mass spectroscopy, 2-D gel electrophoresis, and so forth, all which are known to those of ordinary skill in the art.

Assays for producing enzyme targets, testing their activity, and conducting screens for their inhibition or activation are described below using examples of enzymes related to fatty acid biosynthesis. These assays can be adapted by one of ordinary skill in the art, or other assays known in the art can be used, to test the activity of other targets of the invention.

4.1 High Throughput Screening of Compounds and Target Enzymes

In one embodiment, high throughput screening using, e.g., mass spectrometry can be used to screen a number of compounds and a number of potential target enzymes simultaneously. Mass spectrometry can be utilized for determination of metabolite levels and enzymatic activity.

The levels of specific metabolites (e.g. AMP, ATP) can be quantified by liquid chromatography-mass spectrometry (LC-MS/MS). A metabolite of interest will have a specific chromatographic retention time at which point the mass spectrometer performs a selected reaction monitoring scan event (SRM) that consists of three identifiers:

1) The metabolite's mass (the parent ion);

2) The energy required to fragment the parent ion in a collision with argon to yield a fragment with a specific mass; and

3) The mass of the specific fragment ion.

Utilizing the above identifiers, the accumulation of a metabolite can be measured whose production depends on the activity of a metabolic enzyme of interest. By adding an excess of enzyme substrate to a cellular lysate, so as to make the activity of the enzyme rate limiting, the accumulation of enzymatic product over time is then measured by LC-MS/MS as outlined above, and serves as a function of the metabolic enzyme's activity. An example of such an assay is reported in Munger et al, 2006 PLoS Pathogens, 2: 1-11, incorporated herein by reference in its entirety, in which the activity of phosphofructokinase present in infected lysates was measured by adding an excess of the phosphofructokinase substrates ATP and fructose phosphate and measuring fructose bisphosphatc accumulation by LC-MS/MS. This approach can be adopted to measure the activities of numerous host target enzymes.

4.2 Kinetic Flux Profiling (KFP) to Assess Potential Antiviral Compounds

In a further embodiment of the invention, cellular metabolic fluxes are profiled in the presence or absence of a virus using kinetic flux profiling (KFP) (See Munger et al. 2008 Nature Biotechnology, 26: 1179-1186) in the presence or absence of a compound found to inhibit a target enzyme in one of the aforementioned assays. Such metabolic flux profiling provides additional (i) guidance about which components of a host's metabolism can be targeted for antiviral intervention; (ii) guidance about the metabolic pathways targeted by different viruses; and (iii) validation of compounds as potential antiviral agents based on their ability to offset the metabolic flux caused by a virus or trigger cell-lethal metabolic derangements specifically in virally infected cells. In one embodiment, the kinetic flux profiling methods of the invention can be used for screening to determine (i) the specific alterations in metabolism caused by different viruses and (ii) the ability of a compound to offset (or specifically augment) alterations in metabolic flux caused by different viruses.

Thus, in one embodiment of the invention, cells are infected with a virus and metabolic flux is assayed at different time points after virus infection, such time points known to one of skill in the art. For example, for HCMV, flux can be measured 24, 48, or 72 hours post-infection, whereas for a faster growing virus like HSV, flux can be measured at 6, 12, or 18 hours post-infection. If the metabolic flux is altered in the presence of the virus, then the virus alters cellular metabolism during infection. The type of metabolic flux alteration observed (See above and examples herein) will provide guidance as to the cellular pathways that the virus acts on. Assays well known to those of skill in the art and described herein below can then be employed to confirm the target of the virus. Similarly, compounds can be tested for the ability to interfere with the virus in the assays for antiviral activity described in Section 5 below. If it appears that a virus modulates the level and/or activity of a particular enzyme, inhibitors of that enzyme can be tested for their antiviral effect. If well-characterized compounds are observed to be effective antivirals, other compounds that modulate the same target can similarly be assessed as potential antivirals.

In one embodiment of the invention, a virus infected cell is contacted with a compound and metabolic flux is measured. If the metabolic flux in the presence of the compound is different from the metabolic flux in the absence of the compound, in a manner wherein the metabolic effects of the virus have been inhibited or augmented, then a compound that modulates the virus' ability to alter the metabolic flux has been identified. The type of metabolic flux alteration observed will provide guidance as to the cellular pathway that the compound is acting on. Assays well known to those of skill in the art and described herein can then be employed to confirm the target of the antiviral compound.

In one embodiment, high throughput metabolome quantitation mass spectrometry can be used to screen for changes in metabolism caused by infection of a virus and whether or not a compound or library of compounds offsets these changes. See Munger et al. 2006. PLoS Pathogens, 2: 1-11.

4.3 Identification of Compounds

Using metabolome and fluxome-based analysis of virus infected cells, the inventors have identified host cell target enzymes listed and demonstrated that virus replication can be reduced by reducing expression of the target enzymes. Further, any compound of interest can be tested for its ability to modulate the activity of these enzymes. Alternatively, compounds can be tested for their ability to inhibit any other host cell enzyme related to metabolism. Once such compounds are identified as having metabolic enzyme—modulating activity, they can be further tested for their antiviral activity as described in Section 5. Alternatively, compounds can be screened for antiviral activity and optionally characterized using the metabolic screening assays described herein.

In one embodiment, high throughput screening methods are used to provide a combinatorial chemical or peptide library (e.g., a publicly available library) containing a large number of potential therapeutic compounds (potential modulators or ligand compounds). Such “combinatorial chemical libraries” or “ligand libraries” are then screened in one or more assays, as described in Section 2 herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (See, e.g., U.S. Pat. No. 5,010,175, Furka, Int. J. Pcpt. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (See Ausubel, Berger and Sambrook, all supra), peptide nucleic acid libraries (See, e.g., U.S. Pat. No. 5,539,083), antibody libraries (See, e.g., Vaughn et al., Nature Biotechnology, 14(3):309-314 (1996) and PCT/US96/10287), carbohydrate libraries (See, e.g., Liang et al., Science, 274:1520-1522 (1996) and International Patent Application Publication NO. WO 1997/000271), small organic molecule libraries (See, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., scaffold or framework). Devices for the preparation of combinatorial libraries are commercially available (See, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (See, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.). The test compounds can also be obtained from: biological libraries; peptoid libraries (libraries of molecules having the functionalities of peptides, but with a novel, non-peptide backbone which are resistant to enzymatic degradation but which nevertheless remain bioactive; See, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library). Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.). Enzymes can be screened for identifying compounds which can be selected from a combinatorial chemical library or any other suitable source (Hogan, Jr., Nat. Biotechnology 15:328, 1997).

Any assay herein, e.g., an in vitro assay or an in vivo assay, can be performed individually, e.g., just with the test compound, or with appropriate controls. For example, a parallel assay without the test compound, or other parallel assays without other reaction components, e.g., without a target or without a substrate. Alternatively, it is possible to compare assay results to a reference, e.g., a reference value, e.g., obtained from the literature, a prior assay, and so forth. Appropriate correlations and art known statistical methods can be used to evaluate an assay result. See Section 4.1 above.

Once a compound is identified as having a desired effect, production quantities of the compound can be synthesized, e.g., producing at least 50 mg, 500 mg, 5 g, or 500 g of the compound. Although a compound that is able to penetrate a host cell is preferable in the practice of the invention, a compound may be combined with solubilizing agents or administered in combination with another compound or compounds to maintain its solubility, or help it enter a host cell, e.g., by mixture with lipids. The compound can be formulated, e.g., for administration to a subject, and may also be administered to the subject.

### 5. Characterization of Antiviral Activity of Compounds

5.1 Viruses

The present invention provides compounds for use in the prevention, management and/or treatment of viral infection. The antiviral activity of compounds against any virus can be tested using techniques described in Section 5.2 herein below. The virus may be enveloped or naked, have a DNA or RNA genome, or have a double-stranded or single-stranded genome. See, e.g., FIG. 1 modified from Flint et al., Principles of Virology: Molecular Biology, Pathogenesis and Control of Animal Viruses. 2nd edition, ASM Press, 2003, for a subset of virus families and their classification, as well as a subset of viruses against which compounds can be assessed for antiviral activity. In specific embodiments, the virus infects human. In other embodiments, the virus infects non-human animals. In a specific embodiment, the virus infects pigs, fowl, other livestock, or pets.

In certain embodiments, the virus is an enveloped virus. Enveloped viruses include, but are not limited to viruses that are members of the hepadnavirus family, herpesvirus family, iridovirus family, poxvirus family, flavivirus family, togavirus family, retrovirus family, coronavirus family, Filovirus family, rhabdovirus family, bunyavirus family, orthomyxovirus family, paramyxovirus family, and arenavirus family. Non-limiting examples of viruses that belong to these families are included in Table 3.

TABLE 3
Families of Enveloped Viruses

| Virus Family | Members |
|---|---|
| Hepadnavirus | hepatitis B virus (HBV), woodchuck hepatitis virus, ground squirrel |
| (Hepadnaviridae) | hepatitis virus, duck hepatitis B virus, heron hepatitis B virus |
| Herpesvirus | herpes simplex virus (HSV) types 1 and 2, varicella-zoster virus, |
| (Herpesviridae) | cytomegalovirus (CMV), human cytomegalovirus (HCMV), Epstein- |
| | Barr virus (EBV), human herpesvirus 6 (variants A and B), human |
| | herpesvirus 7, human herpesvirus 8, Kaposi's sarcoma-associated |
| | herpes virus (KSHV), B virus |
| Poxvirus | vaccinia virus, variola virus, smallpox virus, monkeypox virus, |
| (Poxviridae) | cowpox virus, camelpox virus, mousepox virus, raccoonpox viruses, |
| | molluscum contagiosum virus, orf virus, milker's nodes virus, bovin |
| | papullar stomatitis virus, sheeppox virus, goatpox virus, lumpy skin |
| | disease virus, fowlpox virus, canarypox virus, pigeonpox virus, |
| | sparrowpox virus, myxoma virus, hare fibroma virus, rabbit fibroma |
| | virus, squirrel fibroma viruses, swinepox virus, tanapox virus, |
| | Yabapox virus |
| Flavivirus | dengue virus, hepatitis C virus (HCV), GB hepatitis viruses (GBV-A, |
| (Flaviviridae) | GBV-B and GBV-C), West Nile virus, yellow fever virus, St.Louis |
| | encephalitis virus, Japanese encephalitis virus, Powassan virus, tick- |
| | borne encephalitis virus, Kyasanur Forest disease virus |
| Togavirus | Venezuelan equine encephalitis virus, chikungunya virus, Ross River |
| (Togaviridae) | virus, Mayaro virus, Sindbis virus, rubella virus |
| Retrovirus | human immunodeficiency virus (HIV) types 1 and 2, human T cell |
| (Retroviridae) | leukemia virus (HTLV) types 1, 2, and 5, mouse mammary tumor |
| | virus (MMTV), Rous sarcoma virus (RSV), lentiviruses |
| Coronavirus | severe acute respiratory syndrome (SARS) virus |
| (Coronaviridae) | |
| Filovirus | Ebola virus, Marburg virus |
| (Filoviridae) | |
| Rhabdovirus | rabies virus, vesicular stomatitis virus |
| (Rhabdoviridae) | |
| Bunyavirus | Crimean-Congo hemorrhagic fever virus, Rift Valley fever virus, La |
| (Bunyaviridae) | Crosse virus, Hantaan virus |
| Orthomyxovirus | influenza virus (types A, B, and C) |
| (Orthomyxoviridae) | |
| Paramyxovirus | parainfluenza virus, respiratory syncytial virus (types A and B), |
| (Paramyxoviridae) | measles virus, mumps virus |
| Arenavirus | lymphocytic choriomeningitis virus, Junin virus, Machupo virus, |
| (Arenaviridae) | Guanarito virus, Lassa virus, Ampari virus, Flexal virus, Ippy virus, |
| | Mobala virus, Mopeia virus, Latino virus, Parana virus, Pichinde |
| | virus, Tacaribe virus, Tamiami virus |

In some embodiments, the virus is a non-enveloped virus, i.e., the virus does not have an envelope and is naked. Non-limiting examples of such viruses include viruses that are members of the parvovirus family, circovirus family, polyoma virus family, papillomavirus family, adenovirus family, iridovirus family, reovirus family, bimavirus family, calicivirus family, and picomavirus family. Examples of viruses that belong to these families include, but are not limited to, those set forth in Table 4.

TABLE 4
Families of Non-Enveloped (Naked) Viruses

| Virus Family | Members |
|---|---|
| Parvovirus | canine parvovirus, parvovirus B19 |
| (Parvoviridae) | |
| Circovirus | porcine circovirus type 1 and 2, BFDV (Beak and Feather Disease |
| (Circoviridae) | Virus), chicken anaemia virus |
| Polyomavirus | simian virus 40 (SV40), JC virus, BK virus, Budgerigar fledgling |
| (Polyomaviridae) | disease virus |
| Papillomavirus | human papillomavirus, bovine papillomavirus (BPV) type 1 |
| (Papillomaviridae) | |
| Adenovirus | human adenovirus (HAdV-A, HAdV-B, HAdV-C, HAdV-D, HAdV- |
| (Adenoviridae) | E, and HAdV-F), fowl adenovirus A, ovine adenovirus D, frog |
| | adenovirus |
| Reovirus | human orbivirus, human coltivirus, mammalian orthorcovirus, |
| (Reoviridae) | bluetongue virus, rotavirus A, rotaviruses (groups B to G), Colorado |
| | tick fever virus, aquareovirus A, cypovirus 1, Fiji disease virus, rice |
| | dwarf virus, rice ragged stunt virus, idnoreovirus 1, mycoreovirus 1 |
| Birnavirus | bursal disease virus, pancreatic necrosis virus |
| (Birnaviridae) | |
| Calicivirus | swine vesicular exanthema virus, rabbit hemorrhagic disease virus, |
| (Caliciviridae) | Norwalk virus, Sapporo virus |
| Picornavirus | human polioviruses (1-3), human coxsackieviruses A1-22, 24 (CA1-22 |
| (Picornaviridae) | and CA24, CA23 = echovirus 9), human coxsackieviruses (B1-6 |
| | (CB1-6)), human echoviruses 1-7, 9, 11-27, 29-33, vilyuish virus, |
| | simian enteroviruses 1-18 (SEV1-18), porcine enteroviruses 1-11 |
| | (PEV1-11), bovine enteroviruses 1-2 (BEV1-2), hepatitis A virus, |
| | rhinoviruses, hepatoviruses, cardioviruses, aphthoviruses, echoviruses |

In certain embodiments, the virus is a DNA virus. In other embodiments, the virus is a RNA virus. In one embodiment, the virus is a DNA or a RNA virus with a single-stranded genome. In another embodiment, the virus is a DNA or a RNA virus with a double-stranded genome.

In some embodiments, the virus has a linear genome. In other embodiments, the virus has a circular genome. In some embodiments, the virus has a segmented genome. In other embodiments, the virus has a non-segmented genome.

In some embodiments, the virus is a positive-stranded RNA virus. In other embodiments, the virus is a negative-stranded RNA virus. In one embodiment, the virus is a segmented, negative-stranded RNA virus. In another embodiment, the virus is a non-segmented negative-stranded RNA virus.

In some embodiments, the virus is an icosahedral virus. In other embodiments, the virus is a helical virus. In yet other embodiments, the virus is a complex virus.

In certain embodiments, the virus is a herpes virus, e.g., HSV-1, HSV-2, and CMV. In other embodiments, the virus is not a herpes virus (e.g., HSV-1, HSV-2, and CMV). In a specific embodiment, the virus is HSV. In an alternative embodiment, the virus is not HSV. In another embodiment, the virus is HCMV. In a further alternative embodiment, the virus is not HCMV. In another embodiment, the virus is a liver trophic virus. In an alternative embodiment, the virus is not a liver trophic virus. In another embodiment, the virus is a hepatitis virus. In an alternate embodiment, the virus is not a hepatitis virus. In another embodiment, the virus is a hepatitis C virus. In a further alternative embodiment, the virus is not a hepatitis C virus. In another specific embodiment, the virus is an influenza virus. In an alternative embodiment, the virus is not an influenza virus. In some embodiments, the virus is a retrovirus. In some embodiments, the virus is not a retrovirus. In some embodiments, the virus is HIV. In other embodiments, the virus is not HIV. In certain embodiments, the virus is a hepatitis B virus. In another alternative embodiment, the virus is not a hepatitis B virus. In a specific embodiment, the virus is EBV. In a specific alternative embodiment, the virus is not EBV. In some embodiments, the virus is Kaposi's sarcoma-associated herpes virus (KSHV). In some alternative embodiments, the virus is not KSHV. In certain embodiments the virus is a variola virus. In certain alternative embodiments, the virus is not variola virus. In one embodiment, the virus is a Dengue virus. In one alternative embodiment, the virus is not a Dengue virus. In other embodiments, the virus is a SARS virus. In other alternative embodiments, the virus is not a SARS virus. In a specific embodiment, the virus is an Ebola virus. In an alternative embodiment, the virus is not an Ebola virus. In some embodiments the virus is a Marburg virus. In an alternative embodiment, the virus is not a Marburg virus. In certain embodiments, the virus is a measles virus. In some alternative embodiments, the virus is not a measles virus. In particular embodiments, the virus is a vaccinia virus. In alternative embodiments, the virus is not a vaccinia virus. In some embodiments, the virus is varicella-zoster virus (VZV). In an alternative embodiment the virus is not VZV. In some embodiments, the virus is a picornavirus. In alternative embodiments, the virus is not a picornavirus. In certain embodiments the virus is not a rhinovirus. In certain embodiments, the virus is a poliovirus. In alternative embodiments, the virus is not a poliovirus. In some embodiments, the virus is an adenovirus. In alternative embodiments, the virus is not adenovirus. In particular embodiments, the virus is a coxsackievirus (e.g., coxsackievirus B3). In other embodiments, the virus is not a coxsackievirus (e.g., coxsackievirus B3). In some embodiments, the virus is a rhinovirus. In other embodiments, the virus is not a rhinovirus. In certain embodiments, the virus is a human papillomavirus (HPV). In other embodiments, the virus is not a human papillomavirus. In certain embodiments, the virus is a virus selected from the group consisting of the viruses listed in Tables 3 and 4. In other embodiments, the virus is not a virus selected from the group consisting of the viruses listed in Tables 3 and 4. In one embodiment, the virus is not one or more viruses selected from the group consisting of the viruses listed in Tables 3 and 4.

The antiviral activities of compounds against any type, subtype or strain of virus can be assessed. For example, the antiviral activity of compounds against naturally occurring strains, variants or mutants, mutagenized viruses, reassortants and/or genetically engineered viruses can be assessed.

The lethality of certain viruses, the safety issues concerning working with certain viruses and/or the difficulty in working with certain viruses may preclude (at least initially) the characterization of the antiviral activity of compounds on such viruses. Under such circumstances, other animal viruses that are representative of such viruses may be utilized. For example, SIV may be used initially to characterize the antiviral activity of compounds against HIV. Further, Pichinde virus may be used initially to characterize the antiviral activity of compounds against Lassa fever virus.

In some embodiments, the virus achieves peak titer in cell culture or a subject in 4 hours or less, 6 hours or less, 8 hours or less, 12 hours or less, 16 hours or less, or 24 hours or less. In other embodiments, the virus achieves peak titers in cell culture or a subject in 48 hours or less, 72 hours or less, or 1 week or less. In other embodiments, the virus achieves peak titers after about more than 1 week. In accordance with these embodiments, the viral titer may be measured in the infected tissue or serum.

In some embodiments, the virus achieves in cell culture a viral titer of 10⁴ pfu/ml or more, 5×10⁴ pfu/ml or more, 10⁵ pfu/ml or more, 5×10⁵ pfu/ml or more, 10⁶ pfu/ml or more, 5×10⁶ pfu/ml or more, 10⁷ pfu/ml or more, 5×10⁷ pfu/ml or more, 10⁸ pfu/ml or more, 5×10⁸ pfu/ml or more, 10⁹ pfu/ml or more, 5×10⁹ pfu/ml or more, or 10¹⁰ pfu/ml or more. In certain embodiments, the virus achieves in cell culture a viral titer of 10⁴ pfu/ml or more, 5×10⁴ pfu/ml or more, 10⁵ pfu/ml or more, 5×10⁵ pfu/ml or more, 10⁶ pfu/ml or more, 5×10⁶ pfu/ml or more, 10⁷ pfu/ml or more, 5×10⁷ pfu/ml or more, 10⁸ pfu/ml or more, 5×10⁸ pfu/ml or more, 10⁹ pfu/ml or more, 5×10⁹ pfu/ml or more, or 10¹⁰ pfu/ml or more within 4 hours, 6 hours, 8 hours, 12 hours, 16 hours, or 24 hours or less. In other embodiments, the virus achieves in cell culture a viral titer of 10⁴ pfu/ml or more, 5×10⁴ pfu/ml or more, 10⁵ pfu/ml or more, 5×10⁵ pfu/ml or more, 10⁶ pfu/ml or more, 5×10⁶ pfu/ml or more, 10⁷ pfu/ml or more, 5×10⁷ pfu/ml or more, 10⁸ pfu/ml or more, 5×10⁸ pfu/ml or more, 10⁹ pfu/ml or more, 5×10⁹ pfu/ml or more, or 10¹⁰ pfu/ml or more within 48 hours, 72 hours, or 1 week.

In some embodiments, the virus achieves a viral yield of 1 pfu/ml or more, 10 pfu/ml or more, 5×10¹ pfu/ml or more, 10² pfu/ml or more, 5×10² pfu/ml or more, 10³ pfu/ml or more, 2.5×10³ pfu/ml or more, 5×10³ pfu/ml or more, 10⁴ pfu/ml or more, 2.5×10⁴ pfu/ml or more, 5×10⁴ pfu/ml or more, or 10⁵ pfu/ml or more in a subject. In certain embodiments, the virus achieves a viral yield of 1 pfu/ml or more, 10 pfu/ml or more, 5×10¹ pfu/ml or more, 10² pfu/ml or more, 5×10² pfu/ml or more, 10³ pfu/ml or more, 2.5×10³ pfu/ml or more, 5×10³ pfu/ml or more, 10⁴ pfu/ml or more, 2.5×10⁴ pfu/ml or more, 5×10⁴ pfu/ml or more, or 10⁵ pfu/ml or more in a subject within 4 hours, 6 hours, 8 hours, 12 hours, 16 hours, 24 hours, or 48 hours. In certain embodiments, the virus achieves a viral yield of 1 pfu/ml or more, 10 pfu/ml or more, 10¹ pfu/ml or more, 5×10¹ pfu/ml or more, 10² pfu/ml or more, 5×10² pfu/ml or more, 10³ pfu/ml or more, 2.5×10³ pfu/ml or more, 5×10³ pfu/ml or more, 10⁴ pfu/ml or more, 2.5×10⁴ pfu/ml or more, 5×10⁴ pfu/ml or more, or 10⁵ pfu/ml or more in a subject within 48 hours, 72 hours, or 1 week. In accordance with these embodiments, the viral yield may be measured in the infected tissue or serum. In a specific embodiment, the subject is immunocompetent. In another embodiment, the subject is immunocompromised or immunosuppressed.

In some embodiments, the virus achieves a viral yield of 1 pfu or more, 10 pfu or more, 5×10¹ pfu or more, 10² pfu or more, 5×10² pfu or more, 10³ pfu or more, 2.5×10³ pfu or more, 5×10³ pfu or more, 10⁴ pfu or more, 2.5×10⁴ pfu or more, 5×10⁴ pfu or more, or 10⁵ pfu or more in a subject. In certain embodiments, the virus achieves a viral yield of 1 pfu or more, 10 pfu or more, 5×10¹ pfu or more, 10² pfu or more, 5×10² pfu or more, 10³ pfu or more, 2.5×10³ pfu or more, 5×10³ pfu or more, 10⁴ pfu or more, 2.5×10⁴ pfu or more, 5×10⁴ pfu or more, or 10⁵ pfu or more in a subject within 4 hours, 6 hours, 8 hours, 12 hours, 16 hours, 24 hours, or 48 hours. In certain embodiments, the virus achieves a viral yield of 1 pfu or more, 10 pfu or more, 10¹ pfu or more, 5×10¹ pfu or more, 10² pfu or more, 5×10² pfu or more, 10³ pfu or more, 2.5×10³ pfu or more, 5×10³ pfu or more, 10⁴ pfu or more, 2.5×10⁴ pfu or more, 5×10⁴ pfu or more, or 10⁵ pfu or more in a subject within 48 hours, 72 hours, or 1 week. In accordance with these embodiments, the viral yield may be measured in the infected tissue or serum. In a specific embodiment, the subject is immunocompetent. In another embodiment, the subject is immunocompromised or immunosuppressed.

In some embodiments, the virus achieves a viral yield of 1 infectious unit or more, 10 infectious units or more, 5×10¹ infectious units or more, 10² infectious units or more, 5×10² infectious units or more, 10³ infectious units or more, 2.5×10³ infectious units or more, 5×10³ infectious units or more, 10⁴ infectious units or more, 2.5×10⁴ infectious units or more, 5×10⁴ infectious units or more, or 10⁵ infectious units or more in a subject. In certain embodiments, the virus achieves a viral yield of 1 infectious unit or more, 10 infectious units or more, 5×10¹ infectious units or more, 10² infectious units or more, 5×10² infectious units or more, 10³ infectious units or more, 2.5×10³ infectious units or more, 5×10³ infectious units or more, 10⁴ infectious units or more, 2.5×10⁴ infectious units or more, 5×10⁴ infectious units or more, or 10⁵ infectious units or more in a subject within 4 hours, 6 hours, 8 hours, 12 hours, 16 hours, 24 hours, or 48 hours. In certain embodiments, the virus achieves a viral yield of 1 infectious unit or more, 10 infectious units or more, 10¹ infectious units or more, 5×10¹ infectious units or more, 10² infectious units or more, 5×10² infectious units or more, 10³ infectious units or more, 2.5×10³ infectious units or more, 5×10³ infectious units or more, 10⁴ infectious units or more, 2.5×10⁴ infectious units or more, 5×10⁴ infectious units or more, or 10⁵ infectious units or more in a subject within 48 hours, 72 hours, or 1 week. In accordance with these embodiments, the viral yield may be measured in the infected tissue or serum. In a specific embodiment, the subject is immunocompetent. In another embodiment, the subject is immunocompromised or immunosuppressed. In a specific embodiment, the virus achieves a yield of less than 10⁴ infectious units. In other embodiments the virus achieves a yield of 10⁵ or more infectious units.

In some embodiments, the virus achieves a viral titer of 1 infectious unit per ml or more, 10 infectious units per ml or more, 5×10¹ infectious units per ml or more, 10² infectious units per ml or more, 5×10² infectious units per ml or more, 10³ infectious units per ml or more, 2.5×10³ infectious units per ml or more, 5×10³ infectious units per ml or more, 10⁴ infectious units per ml or more, 2.5×10⁴ infectious units per ml or more, 5×10⁴ infectious units per ml or more, or 10⁵ infectious units per ml or more in a subject. In certain embodiments, the virus achieves a viral titer of 10 infectious units per ml or more, 5×10¹ infectious units per ml or more, 10² infectious units per ml or more, 5×10² infectious units per ml or more, 10³ infectious units per ml or more, 2.5×10³ infectious units per ml or more, 5×10³ infectious units per ml or more, 10⁴ infectious units per ml or more, 2.5×10⁴ infectious units per ml or more, 5×10⁴ infectious units per ml or more, or 10⁵ infectious units per ml or more in a subject within 4 hours, 6 hours, 8 hours, 12 hours, 16 hours, 24 hours, or 48 hours. In certain embodiments, the virus achieves a viral titer of 1 infectious unit per mL or more, 10 infectious units per ml or more, 5×10¹ infectious units per ml or more, 10² infectious units per ml or more, 5×10² infectious units per ml or more, 10³ infectious units per mL or more, 2.5×10³ infectious units per ml or more, 5×10³ infectious units per ml or more, 10⁴ infectious units per ml or more, 2.5×10⁴ infectious units per ml or more, 5×10⁴ infectious units per ml or more, or 10⁵ infectious units per ml or more in a subject within 48 hours, 72 hours, or 1 week. In accordance with these embodiments, the viral titer may be measured in the infected tissue or serum. In a specific embodiment, the subject is immunocompetent. In another embodiment, the subject is immunocompromised or immunosuppressed. In a specific embodiment, the virus achieves a titer of less than 10⁴ infectious units per ml. In some embodiments, the virus achieves 10⁵ or more infectious units per ml.

In some embodiments, the virus infects a cell and produces, 10¹ or more, 2.5×10¹ or more, 5×10¹ or more, 7.5×10¹ or more, 10² or more, 2.5×10² or more, 5×10² or more, 7.5×10² or more, 10³ or more, 2.5×10³ or more, 5×10³ or more, 7.5×10³ or more, 10⁴ or more, 2.5×10⁴ or more, 5×10⁴ or more, 7.5×10⁴ or more, or 10⁵ or more viral particles per cell. In certain embodiments, the virus infects a cell and produces 10 or more, 10¹ or more, 2.5×10¹ or more, 5×10¹ or more, 7.5×10¹ or more, 10² or more, 2.5×10² or more, 5×10² or more, 7.5×10² or more, 10³ or more, 2.5×10³ or more, 5×10³ or more, 7.5×10³ or more, 10⁴ or more, 2.5×10⁴ or more, 5×10⁴ or more, 7.5×10⁴ or more, or 10⁵ or more viral particles per cell within 4 hours, 6 hours, 8 hours, 12 hours, 16 hours, or 24 hours. In other embodiments, the virus infects a cell and produces 10 or more, 10¹ or more, 2.5×10¹ or more, 5×10¹ or more, 7.5×10¹ or more, 10² or more, 2.5×10² or more, 5×10² or more, 7.5×10² or more, 10³ or more, 2.5×10³ or more, 5×10³ or more, 7.5×10³ or more, 10⁴ or more, 2.5×10⁴ or more, 5×10⁴ or more, 7.5×10⁴ or more, or 10⁵ or more viral particles per cell within 48 hours, 72 hours, or 1 week.

In other embodiments, the virus is latent for a period of about at least 1 day, 2 days, 3 days, 4 days, 5 days, 6 days, 7 days, 8 days, 9 days, 10 days, 11 days, 12 days, 13 days, 14 days, or 15 days. In another embodiment, the virus is latent for a period of about at least 1 week, or 2 weeks, 3 weeks, 4 weeks, 5 weeks, 6 weeks, 7 weeks, 8 weeks, 9 weeks, or 10 weeks. In a further embodiment, the virus is latent for a period of about at least 1 month, 2 months, 3 months, 4 months, 5 months, 6 months, 7 months, 8 months, 9 months, 10 months, or 11 months. In yet another embodiment, the virus is latent for a period of about at least 1 year, 2 years, 3 years, 4 years, 5 years, 6 years, 7 years, 8 years, 9 years, 10 years, 11 years, 12 years, 13 years, 14 years, or 15 years. In some embodiments, the virus is latent for a period of greater than 15 years.

5.2 In Vitro Assays to Detect Antiviral Activity

The antiviral activity of compounds may be assessed in various in vitro assays described herein or others known to one of skill in the art. Non-limiting examples of the viruses that can be tested for compounds with antiviral activities against such viruses are provided in Section 5.1, supra. In specific embodiments, compounds exhibit an activity profile that is consistent with their ability to inhibit viral replication while maintaining low toxicity with respect to eukaryotic cells, preferably mammalian cells. For example, the effect of a compound on the replication of a virus may be determined by infecting cells with different dilutions of a virus in the presence or absence of various dilutions of a compound, and assessing the effect of the compound on, e.g., viral replication, viral genome replication, and/or the synthesis of viral proteins. Alternatively, the effect of a compound on the replication of a virus may be determined by contacting cells with various dilutions of a compound or a placebo, infecting the cells with different dilutions of a virus, and assessing the effect of the compound on, e.g., viral replication, viral genome replication, and/or the synthesis of viral proteins. Altered viral replication can be assessed by, e.g., plaque formation. The production of viral proteins can be assessed by, e.g., ELISA, Western blot, immunofluorescence, or flow cytometry analysis. The production of viral nucleic acids can be assessed by, e.g., RT-PCR, PCR, Northern blot analysis, or Southern blot.

In certain embodiments, compounds reduce the replication of a virus by approximately 10%, preferably 15%, 25%, 30%, 45%, 50%, 60%, 75%, 95% or more relative to a negative control (e.g., PBS, DMSO) in an assay described herein or others known to one of skill in the art. In some embodiments, compounds reduce the replication of a virus by about at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to a negative control (e.g., PBS, DMSO) in an assay described herein or others known to one of skill in the art. In other embodiments, compounds reduce the replication of a virus by about at least 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to a negative control (e.g., PBS, DMSO) in an assay described herein or others known to one of skill in the art. In other embodiments, compounds reduce the replication of a virus by about 1 log, 1.5 logs, 2 logs, 2.5 logs, 3 logs, 3.5 logs, 4 logs, 4.5 logs, 5 logs or more relative to a negative control (e.g., PBS, DMSO) in an assay described herein or others known to one of skill in the art. In accordance with these embodiments, such compounds may be further assessed for their safety and efficacy in assays such as those described in Section 5, infra.

In certain embodiments, compounds reduce the replication of a viral genome by approximately 10%, preferably 15%, 25%, 30%, 45%, 50%, 60%, 75%, 95% or more relative to a negative control (e.g., PBS, DMSO) in an assay described herein or others known to one of skill in the art. In some embodiments, compounds reduce the replication of a viral genome by about at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to a negative control (e.g., PBS, DMSO) in an assay described herein or others known to one of skill in the art. In other embodiments, compounds reduce the replication of a viral genome by about at least 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to a negative control (e.g., PBS, DMSO) in an assay described herein or others known to one of skill in the art. In other embodiments, compounds reduce the replication of a viral genome by about 1 log, 1.5 logs, 2 logs, 2.5 logs, 3 logs, 3.5 logs, 4 logs, 4.5 logs, 5 logs or more relative to a negative control (e.g., PBS, DMSO) in an assay described herein or others known to one of skill in the art. In accordance with these embodiments, such compounds may be further assessed for their safety and efficacy in assays such as those described in Section 5, infra.

In certain embodiments, compounds reduce the synthesis of viral proteins by approximately 10%, preferably 15%, 25%, 30%, 45%, 50%, 60%, 75%, 95% or more relative to a negative control (e.g., PBS, DMSO) in an assay described herein or others known to one of skill in the art. In some embodiments, compounds reduce the synthesis of viral proteins by approximately at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to a negative control (e.g., PBS, DMSO) in an assay described herein or others known to one of skill in the art. In other embodiments, compounds reduce the synthesis of viral proteins by approximately at least 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to a negative control (e.g., PBS, DMSO) in an assay described herein or others known to one of skill in the art. In other embodiments, compounds reduce the synthesis of viral proteins by approximately 1 log, 1.5 logs, 2 logs, 2.5 logs, 3 logs, 3.5 logs, 4 logs, 4.5 logs, 5 logs or more relative to a negative control (e.g., PBS, DMSO) in an assay described herein or others known to one of skill in the art. In accordance with these embodiments, such compounds may be further assessed for their safety and efficacy in assays such as those described in Section 5.3, infra.

In some embodiments, compounds result in about a 1.5 fold or more, 2 fold or more, 3 fold or more, 4 fold or more, 5 fold or more, 6 fold or more, 7 fold or more, 8 fold or more, 9 fold or more, 10 fold or more, 15 fold or more, 20 fold or more, 25 fold or more, 30 fold or more, 35 fold or more, 40 fold or more, 45 fold or more, 50 fold or more, 60 fold or more, 70 fold or more, 80 fold or more, 90 fold or more, or 100 fold or more inhibition/reduction of viral yield per round of viral replication. In certain embodiments, compounds result in about a 2 fold or more reduction inhibition/reduction of viral yield per round of viral replication. In specific embodiments, compounds result in about a 10 fold or more inhibition/reduction of viral yield per round of viral replication.

The in vitro antiviral assays can be conducted using any eukaryotic cell, including primary cells and established cell lines. The cell or cell lines selected should be susceptible to infection by a virus of interest. Non-limiting examples of mammalian cell lines that can be used in standard in vitro antiviral assays (e.g., viral cytopathic effect assays, neutral red update assays, viral yield assay, plaque reduction assays) for the respective viruses are set out in Table 5.

TABLE 5
Examples of Mammalian Cell Lines in Antiviral Assays

| Virus | Cell Line |
|---|---|
| herpes simplex virus (HSV) | primary fibroblasts (MRC-5 cells) |
| | Vero cells |
| human cytomegalovirus | primary fibroblasts (MRC-5 cells) |
| (HCMV) | |
| Influenza | primary fibroblasts (MRC-5 cells) |
| | Madin Darby canine kidney (MDCK) |
| | primary chick embryo |
| | chick kidney |
| | calf kidney |
| | African green monkey kidney (Vero) cells |
| | mink lung |
| | human respiratory epithelia cells |
| hepatitis C virus | Huh7 (or Huh7.7) |
| | Huh7.5 |
| | primary human hepatocytes (PHH) |
| | immortalized human hepatocytes (IHH) |
| HIV-1 | MT-2 cells (T cells) |
| Dengue virus | Vero cells |
| Measles virus | African green monkey kidney (CV-1) cells |
| SARS virus | Vero 76 cells |
| Respiratory syncytial virus | African green monkey kidney |
| | (MA-104) cells |
| Venezuelan equine encephalitis | Vero cells |
| virus | |
| West Nile virus | Vero cells |
| yellow fever virus | Vero cells |
| HHV-6 | Cord Blood Lymphocytes (CBL) |
| | Human T cell lymphoblastoid cell lines |
| | (HSB-2 and SupT-1) |
| HHV-8 | B-cell lymphoma cell line (BCBL-1) |
| EBV | umbilical cord blood lymphocytes |

Sections 5.2.1 to 5.2.7 below provide non-limiting examples of antiviral assays that can be used to characterize the antiviral activity of compounds against the respective virus. One of skill in the art will know how to adapt the methods described in Sections 5.2.1 to 5.2.7 to other viruses by, e.g., changing the cell system and viral pathogen, such as described in Table 5.

5.2.1 Viral Cytopathic Effect (CPE) Assay

CPE is the morphological changes that cultured cells undergo upon being infected by most viruses. These morphological changes can be observed easily in unfixed, unstained cells by microscopy. Forms of CPE, which can vary depending on the virus, include, but are not limited to, rounding of the cells, appearance of inclusion bodies in the nucleus and/or cytoplasm of infected cells, and formation of syncytia, or polykaryocytes (large cytoplasmic masses that contain many nuclei). For adenovirus infection, crystalline arrays of adenovirus capsids accumulate in the nucleus to form an inclusion body.

The CPE assay can provide a measure of the antiviral effect of a compound. In a non-limiting example of such an assay, compounds are serially diluted (e.g. 1000, 500, 100, 50, 10, 1 μg/ml) and added to 3 wells containing a cell monolayer (preferably mammalian cells at 80-100% confluent) of a 96-well plate. Within 5 minutes, viruses are added and the plate sealed, incubated at 37° C. for the standard time period required to induce near-maximal viral CPE (e.g., approximately 48 to 120 hours, depending on the virus and multiplicity of infection). CPE is read microscopically after a known positive control drug is evaluated in parallel with compounds in each test. Non-limiting examples of positive controls are ribavirin for dengue, influenza, measles, respiratory syncytial, parainfluenza, Pichinde, Punta Toro and Venezuelan equine encephalitis viruses; cidofovir for adenovirus; pirodovir for rhinovirus; 6-azauridine for West Nile and yellow fever viruses; and alferon (interferon α-n3) for SARS virus. The data are expressed as 50% effective concentrations or approximated virus-inhibitory concentration, 50% endpoint (EC50) and cell-inhibitory concentration, 50% endpoint (IC₅₀). General selectivity index (“SI”) is calculated as the IC50 divided by the EC50. These values can be calculated using any method known in the art, e.g., the computer software program MacSynergy II by M. N. Prichard, K. R. Asaltine, and C. Shipman, Jr., University of Michigan, Ann Arbor, Mich.

In one embodiment, a compound has an SI of greater than 3, or 4, or 5, or 6, or 7, or 8, or 9, or 10, or 11, or 12, or 13, or 14, or 15, or 20, or 21, or 22, or 23, or 24, or 25, or 30, or 35, or 40, or 45, or 50, or 60, or 70, or 80, or 90, or 100, or 200, or 300, or 400, or 500, 1,000, or 10,000. In some embodiments, a compound has an SI of greater than 10. In a specific embodiment, compounds with an SI of greater than 10 are further assessed in other in vitro and in vivo assays described herein or others known in the art to characterize safety and efficacy.

5.2.2 Neutral Red (NR) Dye Uptake Assay

The NR Dye Uptake assay can be used to validate the CPE inhibition assay (See Section 5.2.1). In a non-limiting example of such an assay, the same 96-well microplates used for the CPE inhibition assay can be used. Neutral red is added to the medium, and cells not damaged by virus take up a greater amount of dye. The percentage of uptake indicating viable cells is read on a microplate autoreader at dual wavelengths of 405 and 540 nm, with the difference taken to eliminate background. (See McManus et al., Appl. Environment. Microbiol. 31:35-38, 1976). An EC50 is determined for samples with infected cells and contacted with compounds, and an IC₅₀ is determined for samples with uninfected cells contacted with compounds.

5.2.3 Virus Yield Assay

Lysed cells and supernatants from infected cultures such as those in the CPE inhibition assay (See section 5.2.1) can be used to assay for virus yield (production of viral particles after the primary infection). In a non-limiting example, these supernatants are serial diluted and added onto monolayers of susceptible cells (e.g., Vero cells). Development of CPE in these cells is an indication of the presence of infectious viruses in the supernatant. The 90% effective concentration (EC₉₀), the test compound concentration that inhibits virus yield by 1 log₁₀, is determined from these data using known calculation methods in the art. In one embodiment, the EC90 of compound is at least 1.5 fold, 2 fold, 3 fold, 4 fold, 5 fold, 6 fold, 7 fold, 8 fold, 9 fold, 10 fold, 20 fold, 30 fold, 40 fold, or 50 fold less than the EC90 of the negative control sample.

5.2.4 Plaque Reduction Assay

In a non-limiting example of such an assay, the virus is diluted into various concentrations and added to each well containing a monolayer of the target mammalian cells in triplicate. The plates are then incubated for a period of time to achieve effective infection of the control sample (e.g., 1 hour with shaking every fifteen minutes). After the incubation period, an equal amount of 1% agarose is added to an equal volume of each compound dilution prepared in 2× concentration. In certain embodiments, final compound concentrations between 0.03 μg/ml to 100 μg/ml can be tested with a final agarose overlay concentration of 0.5%. The drug agarose mixture is applied to each well in 2 ml volume and the plates are incubated for three days, after which the cells are stained with a 1.5% solution of neutral red. At the end of the 4-6 hour incubation period, the neutral red solution is aspirated, and plaques counted using a stereomicroscope. Alternatively, a final agarose concentration of 0.4% can be used. In other embodiments, the plates are incubated for more than three days with additional overlays being applied on day four and on day 8 when appropriate. In another embodiment, the overlay medium is liquid rather than semi-solid.

5.2.5 Virus Titer Assay

In this non-limiting example, a monolayer of the target mammalian cell line is infected with different amounts (e.g., multiplicity of 3 plaque forming units (pfu) or 5 pfu) of virus (e.g., HCMV or HSV) and subsequently cultured in the presence or absence of various dilutions of compounds (e.g., 0.1 μg/ml, 1 μg/ml, 5 μg/ml, or 10 μg/ml). Infected cultures are harvested 48 hours or 72 hours post infection and titered by standard plaque assays known in the art on the appropriate target cell line (e.g., Vero cells, MRCS cells). In certain embodiments, culturing the infected cells in the presence of compounds reduces the yield of infectious virus by at least 1.5 fold, 2, fold, 3, fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 100 fold, 500 fold, or 1000 fold relative to culturing the infected cells in the absence of compounds. In a specific embodiment, culturing the infected cells in the presence of compounds reduces the PFU/ml by at least 10 fold relative to culturing the infected cells in the absence of compounds.

In certain embodiments, culturing the infected cells in the presence of compounds reduces the yield of infectious virus by at least 0.5 log 10, 1 log 10, 1.5 log 10, 2 log 10, 2.5 log 10, 3 log 10, 3.5 log 10, 4 log 10, 4.5 log 10, 5 log 10, 5.5 log 10, 6 log 10, 6.5 log 10, 7 log 10, 7.5 log 10, 8 log 10, 8.5 log 10, or 9 log 10 relative to culturing the infected cells in the absence of compounds. In a specific embodiment, culturing the infected cells in the presence of compounds reduces the yield of infectious virus by at least 1 log 10 or 2 log 10 relative to culturing the infected cells in the absence of compounds. In another specific embodiment, culturing the infected cells in the presence of compounds reduces the yield of infectious virus by at least 2 log 10 relative to culturing the infected cells in the absence of compounds.

5.2.6 Flow Cytometry Assay

Flow cytometry can be utilized to detect expression of virus antigens in infected target cells cultured in the presence or absence of compounds (See, e.g., McSharry et al., Clinical Microbiology Rev., 1994, 7:576-604). Non-limiting examples of viral antigens that can be detected on cell surfaces by flow cytometry include, but are not limited to gB, gC, gC, and gE of HSV; E protein of Japanese encephalitis; virus gp52 of mouse mammary tumor virus; gpI of varicella-zoster virus; gB of HCMV; gp160/120 of HIV; HA of influenza; gp110/60 of HHV-6; and H and F of measles virus. In other embodiments, intracellular viral antigens or viral nucleic acid can be detected by flow cytometry with techniques known in the art.

5.2.7 Genetically Engineered Cell Lines for Antiviral Assays

Various cell lines for use in antiviral assays can be genetically engineered to render them more suitable hosts for viral infection or viral replication and more convenient substrates for rapidly detecting virus-infected cells (See, e.g., Olivo, P. D., Clin. Microbiol. Rev., 1996, 9:321-334). In some aspects, these cell lines are available for testing the antiviral activity of compound on blocking any step of viral replication, such as, transcription, translation, pregenome encapsidation, reverse transcription, particle assembly and release. Nonlimiting examples of genetically engineered cells lines for use in antiviral assays with the respective virus are discussed below.

HepG2-2.2.15 is a stable cell line containing the hepatitis B virus (HBV) ayw strain genome that is useful in identifying and characterizing compounds blocking any step of viral replication, such as, transcription, translation, pregenome encapsidation, reverse transcription, particle assembly and release. In one aspect, compounds can be added to HepG2-2.2.15 culture to test whether compound will reduce the production of secreted HBV from cells utilizing real time quantitative PCR (TaqMan) assay to measure HBV DNA copies. Specifically, confluent cultures of HepG2-2.2.15 cells cultured on 96-well flat-bottomed tissue culture plates and are treated with various concentration of daily doses of compounds. HBV virion DNA in the culture medium can be assessed 24 hours after the last treatment by quantitative blot hybridization or real time quantitative PCR (TaqMan) assay. Uptake of neutral red dye (absorbance of internalized dye at 510 nM [A510]) can be used to determine the relative level of toxicity 24 hours following the last treatment. Values are presented as a percentage of the average A510 values for separate cultures of untreated cells maintained on the same plate. Intracellular HBV DNA replication intermediates can be assessed by quantitative Southern blot hybridization. Intracellular HBV particles can be isolated from the treated HepG2-2.2.15 cells and the pregenomic RNA examined by Southern blot analysis. ELISAs can be used to quantify the amounts of the HBV envelope protein, surface antigen (HBsAg), and secreted c-antigen (HBeAg) released from cultures. Lamivudine (3TC) can be used as a positive assay control. (See Korba & Gerin, Antivir. Res. 19:55-70, 1992).

In one aspect, the cell line Huh7 ET (luc-ubi-neo/ET), which contains a new HCV RNA replicon with a stable luciferase (LUC) reporter, can be used to assay compounds antiviral activity against hepatitis C viral replication (See Krieger, N., V. Lohmann, and R. Bartenschlager J. Virol., 2001, 75:4614-4624). The activity of the LUC reporter is directly proportional to HCV RNA levels and positive control antiviral compounds behave comparably using either LUC or RNA endpoints. Subconfluent cultures of Huh7 ET cells are plated onto 96-well plates, compounds are added to the appropriate wells the next day, and the samples as well as the positive (e.g., human interferon-alpha 2b) and negative control samples are processed 72 hr later when the cells are still subconfluent. The HCV RNA levels can also be assessed using quantitative PCR (TaqMan). In some embodiments, compounds reduce the LUC signal (or HCV RNA levels) by 20%, 35%, 30%, 35%, 40%, 45%, 50%, 55%, 60%, 65%, 70%, 75%, 80%, 90%, or 95% or more relative to the untreated sample controls. In a preferred embodiment, compounds reduce the LUC signal (or HCV RNA levels) by 50% or more relative to the untreated cell controls. Other relevant cell culture models to study HCV have been described, e.g., See Durantel et al., J. Hepatology, 2007, 46:1-5.

The antiviral effect of compound can be assayed against EBV by measuring the level of viral capsid antigen (VCA) production in Daudi cells using an ELISA assay. Various concentrations of compounds are tested (e.g., 50 mg/ml to 0.03 mg/ml), and the results obtained from untreated and compound treated cells are used to calculate an EC50 value. Selected compounds that have good activity against EBV VCA production without toxicity will be tested for their ability to inhibit EBV DNA synthesis.

For assays with HSV, the BHKICP6LacZ cell line, which was stably transformed with the E. coli lacZ gene under the transcriptional control of the HSV-1 UL39 promoter, can be used (See Stabell et al., 1992, Methods 38:195-204). Infected cells are detected using β-galactosidase assays known in the art, e.g., colorimetric assay.

Standard antiviral assays for influenza virus has been described, See, e.g., Sidwell et al., Antiviral Research, 2000, 48:1-16. These assays can also be adapted for use with other viruses.

5.2.8 Approach to Identifying and Measuring Metabolic Fluxes Regulated by Viral Infection and Anti-Viral Compounds

Viruses can alter cellular metabolic activity through a variety of routes. These include affecting transcription, translation, and/or degradation of mRNAs and/or proteins, relocalization of mRNAs and/or proteins, covalent modification of proteins, and allosteric regulation of enzymes or other proteins; and alterations to the composition of protein-containing complexes that modify their activity. The net result of all of these changes is modulation of metabolic fluxes to meet the needs of the virus. Thus, metabolic flux changes represent the ultimate endpoint of the virus' efforts to modulate host cell metabolism. Accordingly, fluxes that are increased by the virus are especially likely to be critical to viral survival and replication and to represent valuable drug targets.

A novel approach has been developed to profile metabolic fluxes. It builds upon an approach to measuring nitrogen metabolic fluxes in E. coli developed by Rabinowitz and colleagues (Yuan et al., 2006, Nat. Chem. Biol. 2:529-530), which is incorporated herein by reference. The essence of this kinetic flux profiling (KFP) approach is as follows:

(1) Cells (either uninfected or infected with virus) are rapidly switched from unlabeled to isotope-labeled nutrient (or vice versa); for the present purposes, preferred nutrients include uniformly or partially ¹³C-labeled or ¹⁵N-labeled glucose, glutamine, glutamate, or related compounds including without limitation pyruvate, lactate, glycerol, acetate, aspartate, arginine, and urea. Labels can include all known isotopes of H, C, N, 0, P, or S, including both stable and radioactive labels. Results are dependent on the interplay between the host cell type and the viral pathogen, including the viral load and time post infection.

(2) Metabolism is quenched at various time points following the isotope-switch (e.g., 0.2, 0.5, 1, 2, 5, 10, 20, 30 min and 1, 2, 4, 8, 12, 16, 24, 36, 48 h or a subset or variant thereof). One convenient means of metabolism quenching is addition of cold (e.g., dry-ice temperature) methanol, although other solvents and temperatures, including also boiling solvents, are possible.

(3) The metabolome, including its extent of isotope labeling, is quantified for each collected sample. One convenient means of such quantitation is extraction of metabolites from the cells followed by liquid chromatography-tandem mass spectrometry (LC-MS/MS) analysis of the extract. Appropriate extraction protocols and LC-MS/MS methods are known in the art. See the following citations, which are herein incorporated by reference (Bajad et al., 2006, J Chromatogr. A 1125:76-88; Bolling and Fiehn, 2005, Plant Physiol. 139:1995-2005; Coulier et al., 2006, Anal Chem. 78:6573-6582; Kimball and Rabinowitz, 2006, Anal Biochem. 358:273-280; Lu et al., 2006, J. Am. Soc. Mass Spectrom. 17:37-50; Lu et al., 2007, J Am Soc Mass Spectrom. 18:898-909; Luo et al., 2007, J. Chromatogr. A 1147:153-164; Maharjan and Ferenci, 2003, Anal Biochem 313:145-154; Milne et al., 2006, Methods 39:92-103; Munger et al., 2006, PLoS Pathog. 2:e132; Olsson et al., 2004, Anal Chem. 76:2453-2461; Rabinowitz and Kimball, 2007, Anal Chem. 79:6167-73; Schaub et al., 2006, Biotechnol. Prog. 22:1434-1442; van Winden et al., 2005, FEMS Yeast Research 5:559-568; Villas-Boas et al., 2005, Yeast 22:1155-1169; Wittmann et al., 2004, Anal Biochem. 327:135-139; Wu et al., 2005, Anal Biochem. 336:164-171; Yuan et al., 2006, Nat. Chem. Biol. 2:529-530).

(4) The resulting data is analyzed to determine the cellular metabolic fluxes.

The KFP data is analyzed based on the following principles, through whose application those skilled in the art of cellular metabolism can identify flux changes associated with viral infection by comparing results for infected versus uninfected samples:

(1) Metabolites closer to the added nutrient in the metabolic network will become labeled before their downstream products. Thus, the pattern of labeling provides insight into the route taken to forming a particular metabolite. For example, more rapid labeling of oxaloacetate than citrate upon switching cells from unlabeled to uniformly ¹³C-labeled glucose would imply formation of oxaloacetate via phosphoenolpyruvate carboxylase or phosphoenolpyruvate carboxykinase rather than via clockwise turning of the tricarboxylic acid cycle.

(2) The speed of labeling provides insight into the quantitative flux through different metabolic pathways, with fast labeling of a metabolite pool resulting from large flux through that pool and/or low absolute pool size of it. For the ideal case of a well-mixed system in which a nutrient is being directly converted into an intracellular metabolite, instantaneous switching of the nutrient input into isotope-labeled form, without other modulation of the system, results over time in disappearance of the unlabeled metabolite:

dX^{U}/dt=−f_{X}X^{U}/X^{T} Eq. (A)

where X^{T} is the total pool of metabolite X; X^{U} the unlabeled form; and f_{X} is the sum of all fluxes consuming the metabolite. For f_{X} and X^{T} constant (i.e., the system at pseudo-steady-state prior to the isotope switch),

X^{U}/X^{T}=exp(−f_{X}t/X^{T}) Eq. (B)

and f_{X}=X^{T}k_{X} Eq. (C)

where k_{X} is the apparent first-order rate constant for disappearance of the unlabeled metabolite. According to Eq. (C), the total flux through metabolite X can be determined based on two parameters that can be measured directly experimentally: the intracellular pool size of the metabolite and the rate of disappearance of the unlabeled form. While in practice isotope switching is not instantaneous and slightly more complex equations are required, the full differential equations can still often be solved analytically and typically involve only two free parameters, with one of these, k_{X}, directly yielding total metabolic flux as shown above (Yuan et al., 2006, Nat. Chem. Biol. 2:529-530).

In certain cases involving branched and cyclic pathways, however, the mathematics become more complex and use of more sophisticated computational algorithms to facilitate data analysis may be beneficial. The cellular metabolic network can be described by a system of differential equations describing changes in metabolite levels over time (including changes in isotopic labeling patterns). See the following citations, which are hereby incorporated by reference (Reed et al., 2003, Genome Biol. 4:R54; Sauer, 2006, Mol. Syst. Biol. 2:62; Stephanopoulos, 1999, Metab. Eng. 1:1-11; Szyperski et al., 1999, Metab. Eng. 1:189-197; Zupke et al., 1995). Such descriptions, wherein the form of the equations is parallel to Eq. (A) above, can be solved for fluxes fₓ₁, fₓ₂, etc. based on experimentally observed data describing metabolite concentrations and labeling kinetics (X^{T} at pseudo-steady-state and X^{U}/X^{T} as a function of time). One appropriate class of algorithm for obtaining such solutions is described in the following citations, which are hereby incorporated by reference (Feng and Rabitz, 2004, Biophys. J. 86:1270-1281; Feng et al., 2006, J. Phys. Chem. A. Mol. Spectrosc. Kinet. Environ. Gen. Theory 110:7755-7762).

In general, changes in fluxes induced by viral infections occur slowly relative to the turnover of metabolites. Accordingly, the steady-state assumption generally applies to virally perturbed metabolic networks over short to moderate timescales (e.g., for CMV, up to ˜2 h; the exact length of time depends on the nature of the viral pathogen, with more aggressive pathogens generally associated with shorter time scales).

At steady-state, the flux through all steps of a linear metabolic pathway must be equal. Accordingly, if flux through one step of a pathway is markedly increased by viral infection, the flux through the other steps is likely also increased. A complication arises due to branching, however. While the effect of branching is small in the case that the side branches are associated with low relative flux, the possibility of branching (as well as non-steady-state conditions) points to the need for more experimental data than just one measured pathway flux to implicate other pathway steps as viable drug targets. If increased flux is experimentally demonstrated at both steps upstream and downstream of an unmeasured step of the pathway, however, then one can have greatly increased confidence that the flux at the (unmeasured) intermediate step is also increased. Accordingly, herein we consider demonstration of increased flux at both the upstream and downstream steps (but, in selected embodiments, neither individually) to be adequate to validate the intermediate flux (and associated catalyzing enzyme) as a valid antiviral drug target.

5.3 Characterization of Safety and Efficacy of Compounds

The safety and efficacy of compounds can be assessed using technologies known to one of skill in the art. Sections 5.4 and 5.5 below provide non-limiting examples of cytotoxicity assays and animal model assays, respectively, to characterize the safety and efficacy of compounds. In certain embodiments, the cytotoxicity assays described in Section 5.4 are conducted following the in vitro antiviral assays described in Section 5, supra. In other embodiments, the cytotoxicity assays described in Section 5.4 are conducted before or concurrently with the in vitro antiviral assays described in Section 5, supra.

In some embodiments, compounds differentially affect the viability of uninfected cells and cells infected with virus. The differential effect of a compound on the viability of virally infected and uninfected cells may be assessed using techniques such as those described in Section 5.4, infra, or other techniques known to one of skill in the art. In certain embodiments, compounds are more toxic to cells infected with a virus than uninfected cells. In specific embodiments, compounds preferentially affect the viability of cells infected with a virus. Without being bound by any particular concept, the differential effect of a compound on the viability of uninfected and virally infected cells may be the result of the compound targeting a particular enzyme or protein that is differentially expressed or regulated or that has differential activities in uninfected and virally infected cells. For example, viral infection and/or viral replication in an infected host cells may alter the expression, regulation, and/or activities of enzymes and/or proteins. Accordingly, in some embodiments, other compounds that target the same enzyme, protein or metabolic pathway are examined for antiviral activity. In other embodiments, congeners of compounds that differentially affect the viability of cells infected with virus are designed and examined for antiviral activity. Non-limiting examples of antiviral assays that can be used to assess the antiviral activity of compound are provided in Section 5, supra.

5.4 Cytotoxicity Studies

In a preferred embodiment, the cells are animal cells, including primary cells and cell lines. In some embodiments, the cells are human cells. In certain embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; and THP-1, monocytic cells. Other non-limiting examples of cell lines that can be used to test the cytotoxicity of compounds are provided in Table 5.

Many assays well-known in the art can be used to assess viability of cells (infected or uninfected) or cell lines following exposure to a compound and, thus, determine the cytotoxicity of the compound. For example, cell proliferation can be assayed by measuring Bromodeoxyuridine (BrdU) incorporation (See, e.g., Hoshino et al., 1986, Int. J. Cancer 38, 369; Campana et al., 1988, J. Immunol. Meth. 107:79), (3H) thymidine incorporation (See, e.g., Chen, J., 1996, Oncogene 13:1395-403; Jeoung, J., 1995, J. Biol. Chem. 270:18367 73), by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes are given a designation of T (100% toxic), PVH (partially toxic-very heavy—80%), PH (partially toxic—heavy—60%), P (partially toxic—40%), Ps (partially toxic—slight—20%), or 0 (no toxicity—0%), conforming to the degree of cytotoxicity seen. A 50% cell inhibitory (cytotoxic) concentration (IC₅₀) is determined by regression analysis of these data.

Compounds can be tested for in vivo toxicity in animal models. For example, animal models, described herein and/or others known in the art, used to test the antiviral activities of compounds can also be used to determine the in vivo toxicity of these compounds. For example, animals are administered a range of concentrations of compounds. Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

The toxicity and/or efficacy of a compound in accordance with the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. A compound identified in accordance with the invention that exhibits large therapeutic indices is preferred. While a compound identified in accordance with the invention that exhibits toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of a compound identified in accordance with the invention for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high-performance liquid chromatography. Additional information concerning dosage determination is provided in Section 7.4, infra.

5.5 Animal Models

Compounds and compositions are preferably assayed in vivo for the desired therapeutic or prophylactic activity prior to use in humans. For example, in vivo assays can be used to determine whether it is preferable to administer a compound and/or another therapeutic agent. For example, to assess the use of a compound to prevent a viral infection, the compound can be administered before the animal is infected with the virus. In another embodiment, a compound can be administered to the animal at the same time that the animal is infected with the virus. To assess the use of a compound to treat or manage a viral infection, in one embodiment, the compound is administered after a viral infection in the animal. In another embodiment, a compound is administered to the animal at the same time that the animal is infected with the virus to treat and/or manage the viral infection. In a specific embodiment, the compound is administered to the animal more than one time.

Compounds can be tested for antiviral activity against virus in animal models systems including, but are not limited to, rats, mice, chicken, cows, monkeys, pigs, goats, sheep, dogs, rabbits, guinea pigs, etc. In a specific embodiment of the invention, compounds are tested in a mouse model system. Such model systems are widely used and well-known to the skilled artisan.

Animals are infected with virus and concurrently or subsequently treated with a compound or placebo. Samples obtained from these animals (e.g., serum, urine, sputum, semen, saliva, plasma, or tissue sample) can be tested for viral replication via well known methods in the art, e.g., those that measure altered viral replication (as determined, e.g., by plaque formation) or the production of viral proteins (as determined, e.g., by Western blot, ELISA, or flow cytometry analysis) or viral nucleic acids (as determined, e.g., by RT-PCR, northern blot analysis or southern blot). For quantitation of virus in tissue samples, tissue samples are homogenized in phosphate-buffered saline (PBS), and dilutions of clarified homogenates are adsorbed for 1 hour at 37° C. onto monolayers of cells (e.g., Vero, CEF or MDCK cells). In other assays, histopathologic evaluations are performed after infection, preferably evaluations of the organ(s) the virus is known to target for infection. Virus immunohistochemistry can be performed using a viral-specific monoclonal antibody. Non-limiting exemplary animal models described below (Sections 5.5.1-5.5.5) can be adapted for other viral systems.

The effect of a compound on the virulence of a virus can also be determined using in vivo assays in which the titer of the virus in an infected subject administered a compound, the length of survival of an infected subject administered a compound, the immune response in an infected subject administered a compound, the number, duration and/or severity of the symptoms in an infected subject administered a compound, and/or the time period before onset of one or more symptoms in an infected subject administered a compound is assessed. Techniques known to one of skill in the art can be used to measure such effects.

5.5.1 Herpes Simplex Virus (HSV)

Mouse models of herpes simplex virus type 1 or type 2 (HSV-1 or HSV-2) can be employed to assess the antiviral activity of compounds in vivo. BALB/c mice are commonly used, but other suitable mouse strains that are susceptible can also be used. Mice are inoculated by various routes with an appropriate multiplicity of infection of HSV (e.g., 10⁵ pfu of HSV-1 strain E-377 or 4×10⁴ pfu of HSV-2 strain MS) followed by administration of compounds and placebo. For i.p. inoculation, HSV-1 replicates in the gut, liver, and spleen and spreads to the CNS. For i.n. inoculation, HSV-1 replicates in the nasaopharynx and spreads to the CNS. Any appropriate route of administration (e.g., oral, topical, systemic, nasal), frequency and dose of administration can be tested to determine the optimal dosages and treatment regimens using compounds, optionally in combination with other therapies.

In a mouse model of HSV-2 genital disease, intravaginal inoculation of female Swiss Webster mice with HSV-1 or HSV-2 is carried out, and vaginal swabs are obtained to evaluate the effect of therapy on viral replication (See, e.g., Crute et al., Nature Medicine, 2002, 8:386-391). For example, viral titers by plaque assays are determined from the vaginal swabs. A mouse model of HSV-1 using SKH-1 mice, a strain of immunocompetent hairless mice, to study cutaneous lesions is also described in the art (See, e.g., Crute et al., Nature Medicine, 2002, 8:386-391 and Bolger et al., Antiviral Res., 1997, 35:157-165). Guinea pig models of HSV have also been described, See, e.g., Chen et al., Virol. J, 2004 Nov. 23, 1:11. Statistical analysis is carried out to calculate significance (e.g., a P value of 0.05 or less).

5.5.2 HCMV

Since HCMV does not generally infect laboratory animals, mouse models of infection with murine CMV (MCMV) can be used to assay antiviral activity compounds in vivo. For example, a MCMV mouse model with BALB/c mice can be used to assay the antiviral activities of compounds in vivo when administered to infected mice (See, e.g., Kern et al., Antimicrob. Agents Chemother., 2004, 48:4745-4753). Tissue homogenates isolated from infected mice treated or untreated with compounds are tested using standard plaque assays with mouse embryonic fibroblasts (MEFs). Statistical analysis is then carried out to calculate significance (e.g., a P value of 0.05 or less).

Alternatively, human tissue (i.e., retinal tissue or fetal thymus and liver tissue) is implanted into SCID mice, and the mice are subsequently infected with HCMV, preferably at the site of the tissue graft (See, e.g., Kern et al., Antimicrob. Agents Chemother., 2004, 48:4745-4753). The pfu of HCMV used for inoculation can vary depending on the experiment and virus strain. Any appropriate routes of administration (e.g., oral, topical, systemic, nasal), frequency and dose of administration can be tested to determine the optimal dosages and treatment regimens using compounds, optionally in combination with other therapies. Implant tissue homogenates isolated from infected mice treated or untreated with compounds at various time points are tested using standard plaque assays with human foreskin fibroblasts (HFFs). Statistical analysis is then carried out to calculate significance (i.e., a P value of 0.05 or less).

Guinea pig models of CMV to study antiviral agents have also been described, See, e.g., Bourne et al., Antiviral Res., 2000, 47:103-109; Bravo et al., Antiviral Res., 2003, 60:41-49; and Bravo et al, J. Infectious Diseases, 2006, 193:591-597.

5.5.3 Influenza

Animal models, such as ferret, mouse and chicken, developed for use to test antiviral agents against influenza virus have been described, See, e.g., Sidwell et al., Antiviral Res., 2000, 48:1-16; and McCauley et al., Antiviral Res., 1995, 27:179-186. For mouse models of influenza, non-limiting examples of parameters that can be used to assay antiviral activity of compounds administered to the influenza-infected mice include pneumonia-associated death, serum al-acid glycoprotein increase, animal weight, lung virus assayed by hemagglutinin, lung virus assayed by plaque assays, and histopathological change in the lung. Statistical analysis is carried out to calculate significance (e.g., a P value of 0.05 or less).

Nasal turbinates and trachea may be examined for epithelial changes and subepithelial inflammation. The lungs may be examined for bronchiolar epithelial changes and peribronchiolar inflammation in large, medium, and small or terminal bronchioles. The alveoli are also evaluated for inflammatory changes. The medium bronchioles are graded on a scale of 0 to 3+ as follows: 0 (normal: lined by medium to tall columnar epithelial cells with ciliated apical borders and basal pseudostratified nuclei; minimal inflammation); 1+ (epithelial layer columnar and even in outline with only slightly increased proliferation; cilia still visible on many cells); 2+ (prominent changes in the epithelial layer ranging from attenuation to marked proliferation; cells disorganized and layer outline irregular at the luminal border); 3+ (epithelial layer markedly disrupted and disorganized with necrotic cells visible in the lumen; some bronchioles attenuated and others in marked reactive proliferation).

The trachea is graded on a scale of 0 to 2.5+ as follows: 0 (normal: Lined by medium to tall columnar epithelial cells with ciliated apical border, nuclei basal and pseudostratified. Cytoplasm evident between apical border and nucleus. Occasional small focus with squamous cells); 1+ (focal squamous metaplasia of the epithelial layer); 2+(diffuse squamous metaplasia of much of the epithelial layer, cilia may be evident focally); 2.5+ (diffuse squamous metaplasia with very few cilia evident).

Virus immunohistochemistry is performed using a viral-specific monoclonal antibody (e.g. NP-, N- or HN-specific monoclonal antibodies). Staining is graded 0 to 3+ as follows: 0 (no infected cells); 0.5+ (few infected cells); 1+ (few infected cells, as widely separated individual cells); 1.5+ (few infected cells, as widely separated singles and in small clusters); 2+ (moderate numbers of infected cells, usually affecting clusters of adjacent cells in portions of the epithelial layer lining bronchioles, or in small sublobular foci in alveoli); 3+ (numerous infected cells, affecting most of the epithelial layer in bronchioles, or widespread in large sublobular foci in alveoli).

5.5.4 Hepatitis

A HBV transgenic mouse model, lineage 1.3.46 (official designation, Tg[HBV 1.3 genome] Chi46) has been described previously and can be used to test the in vivo antiviral activities of compounds as well as the dosing and administration regimen (See, e.g., Cavanaugh et al., J. Virol., 1997, 71:3236-3243; and Guidotti et al., J. Virol., 1995, 69:6158-6169). In these HBV transgenic mice, a high level of viral replication occurs in liver parenchymal cells and in the proximal convoluted tubules in the kidneys of these transgenic mice at levels comparable to those observed in the infected liver of patients with chronic HBV hepatitis. HBV transgenic mice that have been matched for age (i.e., 6-10 weeks), sex (i.e., male), and levels of hepatitis B surface antigen (HBsAg) in serum can be treated with compounds or placebo followed by antiviral activity analysis to assess the antiviral activity of compounds. Non-limiting examples of assays that can be performed on these mice treated and untreated with compounds include Southern analysis to measure HBV DNA in the liver, quantitative reverse transcriptase PCR (qRT-PCR) to measure HBV RNA in liver, immunoassays to measure hepatitis e antigen (HBeAg) and HBV surface antigen (HBsAg) in the serum, immunohistochemistry to measure HBV antigens in the liver, and quantitative PCR (qPCR) to measure serum HBV DNA. Gross and microscopic pathological examinations can be performed as needed.

Various hepatitis C virus (HCV) mouse models described in the art can be used in assessing the antiviral activities of compounds against HCV infection (See Zhu et al., Antimicrobial Agents and Chemother., 2006, 50:3260-3268; Bright et al., Nature, 2005, 436:973-978; Hsu et al., Nat. Biotechnol., 2003, 21:519-525; Ilan et al., J. Infect. Dis. 2002, 185:153-161; Kneteman et al., Hepatology, 2006, 43:1346-1353; Mercer et al., Nat. Med., 2001, 7:927-933; and Wu et al., Gastroenterology, 2005, 128:1416-1423). For example, mice with chimeric human livers are generated by transplanting normal human hepatocytes into SCID mice carrying a plasminogen activator transgene (Alb-uPA) (See Mercer et al., Nat. Med., 2001, 7:927-933). These mice can develop prolonged HCV infections with high viral titers after inoculation with HCV (e.g., from infected human serum). Thus, these mice can be administered a compound or placebo prior to, concurrently with, or subsequent to HCV infection, and replication of the virus can be confirmed by detection of negative-strand viral RNA in transplanted livers or expression of HCV viral proteins in the transplanted hepatocyte nodules. The statistical significance of the reductions in the viral replication levels are determined.

Another example of a mouse model of HCV involves implantation of the HuH7 cell line expressing a luciferase reporter linked to the HCV subgenome into SCID mice, subcutaneously or directly into the liver (See Zhu et al., Antimicrobial Agents and Chemother., 2006, 50:3260-3268). The mice are treated with a compound or placebo, and whole-body imaging is used to detect and quantify bioluminescence signal intensity. Mice treated with a compound that is effective against HCV have less bioluminescence signal intensity relative to mice treated with placebo or a negative control.

5.5.5 HIV

The safety and efficacy of compounds against HIV can be assessed in vivo with established animal models well known in the art. For example, a Trimera mouse model of HIV-1 infection has been developed by reconstituting irradiated normal BALB/c mice with murine SCID bone marrow and engrafted human peripheral blood mononuclear cells (See Ayash-Rashkovsky et al., FASEB J., 2005, 19:1149-1151). These mice are injected intraperitoneally with T- and M-tropic HIV-1 laboratory strains. After HIV infection, rapid loss of human CD4⁺ T cells, decrease in CD4/CD8 ratio, and increased T cell activation can be observed. A compound can be administered to these mice and standard assays known in the art can be used to determine the viral replication capacity in animals treated or untreated with a compound. Non-limiting examples of such assays include the COBAS AMPLICOR® RT-PCR assay (Roche Diagnostics, Branchberg, N.J.) to determine plasma viral load (HIV-1 RNA copies/ml); active HIV-1 virus replication assay where human lymphocytes recovered from infected Trimera mice were cocultured with target T cells (MT-2 cells) and HIV-dependent syncytia formation was examined; and human lymphocytes recovered from infected Trimera mice were cocultured with cMAGI indicator cells, where HIV-1 LTR driven trans-activation of β-galactosidase was measured. Levels of anti-HIV-1 antibodies produced in these mice can also be measured by ELISA. Other established mouse models described in the art can also be used to test the antiviral activity of compounds in vivo (See, Mosier et al., Semin. Immunol., 1996, 8:255-262; Mosier et al., Hosp. Pract. (Off Ed)., 1996, 31:41-48, 53-55, 59-60; Bonyhadi et al., Mol. Med. Today, 1997, 3:246-253; Jolicoeur et al., Leukemia, 1999, 13:S78-S80; Browning et al., Proc. Natl. Acad. Sci. USA, 1997, 94:14637-14641; and Sawada et al., J. Exp. Med., 1998, 187:1439-1449). A simian immunodeficiency virus (SIV) nonhuman primate model has also been described (See Schito et al., Curr. HIV Res., 2006, 4:379-386).

### 6. Pharmaceutical Compositions

Any compound described or incorporated by referenced herein may optionally be in the form of a composition comprising the compound. The administration of the combinations of compounds described herein may involve administering to the subject of two or more of the compounds in the same dosage form. The administration of the combinations of compounds described herein may also involve administering to the subject two or more of the compounds in separate dosage forms.

In certain embodiments provided herein, compositions (including pharmaceutical compositions) comprise a compound and a pharmaceutically acceptable carrier, excipient, or diluent.

In other embodiments, provided herein are pharmaceutical compositions comprising an effective amount of a compound and a pharmaceutically acceptable carrier, excipient, or diluent. The pharmaceutical compositions are suitable for veterinary and/or human administration.

The pharmaceutical compositions provided herein can be in any form that allows for the composition to be administered to a subject, said subject preferably being an animal, including, but not limited to a human, mammal, or non-human animal, such as a cow, horse, sheep, pig, fowl, cat, dog, mouse, rat, rabbit, guinea pig, etc., and is more preferably a mammal, and most preferably a human.

In a specific embodiment and in this context, the term “pharmaceutically acceptable carrier, excipient or diluent” means a carrier, excipient or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose free compositions can comprise excipients that are well known in the art and are listed, for example, in the U.S. Pharmacopeia (USP) SP (XXI)/NF (XVI). In general, lactose free compositions comprise an active ingredient, a binder/filler, and a lubricant in pharmaceutically compatible and pharmaceutically acceptable amounts. Preferred lactose free dosage forms comprise a compound, microcrystalline cellulose, pre gelatinized starch, and magnesium stearate.

Further provided herein are anhydrous pharmaceutical compositions and dosage forms comprising one or more compounds, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, NY, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous compositions and dosage forms provided herein can be prepared using anhydrous or low moisture containing ingredients and low moisture or low humidity conditions. Compositions and dosage forms that comprise lactose and at least one compound that comprises a primary or secondary amine are preferably anhydrous if substantial contact with moisture and/or humidity during manufacturing, packaging, and/or storage is expected.

An anhydrous composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided herein are compositions and dosage forms that comprise one or more agents that reduce the rate by which a compound will decompose. Such agents, which are referred to herein as “stabilizers,” include, but are not limited to, antioxidants such as ascorbic acid, pH buffers, or salt buffers.

The compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a compound preferably in purified form, together with a suitable amount of carrier so as to provide the form for proper administration to the patient. The formulation should suit the mode of administration. In a preferred embodiment, the compositions or single unit dosage forms are sterile and in suitable form for administration to a subject, preferably an animal subject, more preferably a mammalian subject, and most preferably a human subject.

Compositions provided herein are formulated to be compatible with the intended route of administration. Examples of routes of administration include, but are not limited to, parenteral, e.g., intravenous, intradermal, subcutaneous, oral (e.g., inhalation), intranasal, transdermal (topical), transmucosal, intra-synovial, ophthalmic, and rectal administration. In a specific embodiment, the composition is formulated in accordance with routine procedures as a composition adapted for intravenous, subcutaneous, intramuscular, oral, intranasal, ophthalmic, or topical administration to human beings. In a preferred embodiment, a composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non aqueous liquid suspensions, oil in water emulsions, or a water in oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Pharmaceutical compositions provided herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms provided herein are prepared by combining a compound in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions provided herein is typically present in from about 50 to about 99 weight percent of the pharmaceutical composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms provided herein include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB 0 SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A compound can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased patient compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or agents.

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms provided herein are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non aqueous vehicles such as, but not limited to, corn oil, cottonseed oil, peanut oil, sesame oil, ethyl oleate, isopropyl myristate, and benzyl benzoate.

Agents that increase the solubility of one or more of the compounds provided herein can also be incorporated into the parenteral dosage forms provided herein.

Transdermal, topical, and mucosal dosage forms provided herein include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms provided herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristatc, isopropyl palmitate, mineral oil, and mixtures thereof to form lotions, tinctures, creams, emulsions, gels or ointments, which are non toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with a compound. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxi de; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more compounds. Similarly, the polarity of a solvent carrier, its ionic strength, or tonicity can be adjusted to improve delivery. Agents such as stearates can also be added to pharmaceutical compositions or dosage forms to advantageously alter the hydrophilicity or lipophilicity of one or more compounds so as to improve delivery. In this regard, stearates can serve as a lipid vehicle for the formulation, as an emulsifying agent or surfactant, and as a delivery enhancing or penetration enhancing agent. Different salts, hydrates or solvates of the compounds can be used to further adjust the properties of the resulting composition.

In certain specific embodiments, the compositions are in oral, injectable, or transdermal dosage forms. In one specific embodiment, the compositions are in oral dosage forms. In another specific embodiment, the compositions are in the form of injectable dosage forms. In another specific embodiment, the compositions are in the form of transdermal dosage forms. In one embodiment, the compounds that are part of the combination therapy are administered by different routes of administration. In one embodiment, the compounds are administered by the same route of administration.

### 7. Prophylactic and Therapeutic Methods

The present invention provides methods of preventing, treating and/or managing a viral infection, said methods comprising administering to a subject in need thereof one or more compounds. In a specific embodiment, the invention provides a method of preventing, treating and/or managing a viral infection, said method comprising administering to a subject in need thereof a dose (or doses) of a prophylactically or therapeutically effective amount of one or more compounds or a composition comprising one or more compounds. A compound or a combination of compounds may be used as any line of therapy (e.g., a first, second, third, fourth or fifth line therapy) for a viral infection.

In another embodiment, the invention relates to a method for reversing or redirecting metabolic flux altered by viral infection in a human subject by administering to a human subject in need thereof, an effective amount of one or more compounds or a composition comprising one or more compounds. For example, viral infection can be treated using combinations of the enzyme inhibition compounds that produce beneficial results, e.g., synergistic effect; reduction of side effects; a higher therapeutic index. In one such embodiment, for example, a citrate lyase inhibitor can be used in combination with an Acetyl-CoA Carboxylase (ACC).

The choice of compounds to be used depends on a number of factors, including but not limited to the type of viral infection, health and age of the patient, and toxicity or side effects. For example, treatments that inhibit enzymes required for core ATP production, such as proton ATPase are not preferred unless given in a regimen that compensates for the toxicity; e.g., using a localized delivery system that limits systemic distribution of the drug.

The present invention encompasses methods for preventing, treating, and/or managing a viral infection for which no antiviral therapy is available or for which the subject has been unresponsive to previous therapies. The present invention also encompasses methods for preventing, treating, and/or managing a viral infection as an alternative to other conventional therapies.

The present invention also provides methods of preventing, treating and/or managing a viral infection, said methods comprising administering to a subject in need thereof one or more of the compounds and one or more other therapies (e.g., prophylactic or therapeutic agents). In a specific embodiment, the other therapies are currently being used, have been used or are known to be useful in the prevention, treatment and/or management of a viral infection. Non-limiting examples of such therapies are provided, for example, in Section 7, infra. In a specific embodiment, one or more compounds are administered to a subject in combination with one or more of the therapies described in Section 7, infra. In another embodiment, one or more compounds are administered to a subject in combination with a supportive therapy, a pain relief therapy, or other therapy that does not have antiviral activity.

The combination therapies of the invention can be administered sequentially and/or concurrently. In one embodiment, the combination therapies of the invention comprise a compound and at least one other therapy which has the same mechanism of action. In another embodiment, the combination therapies of the invention comprise a compound and at least one other therapy which has a different mechanism of action than the compound.

In a specific embodiment, the combination therapies of the present invention improve the prophylactic and/or therapeutic effect of a compound by functioning together with the compound to have an additive or synergistic effect. In another embodiment, the combination therapies of the present invention reduce the side effects associated with each therapy taken alone.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The administered prophylactic and/or therapeutic agents may be administered to a subject by the same or different routes of administration. One or more compounds that are administered to the subject may be administered before or after the other compound or compounds, such that the administration of one compound is separated from administration of the second compound by hours, days or weeks. Alternatively, the administered compounds may be administered to the patient at about the same time.

7.1 Patient Population

According to the invention, compounds, compositions comprising a compound, or a combination therapy is administered to a subject suffering from a viral infection. In other embodiments, compounds, compositions comprising a compound, or a combination therapy is administered to a subject predisposed or susceptible to a viral infection. In some embodiments, compounds, compositions comprising a compound, or a combination therapy is administered to a subject that lives in a region where there has been or might be an outbreak with a viral infection. In some embodiments, the viral infection is a latent viral infection. In one embodiment, a compound or a combination therapy is administered to a human infant. In one embodiment, a compound or a combination therapy is administered to a premature human infant. In other embodiments, the viral infection is an active infection. In yet other embodiments, the viral infection is a chronic viral infection. Non-limiting examples of types of virus infections include infections caused by those provided in Section 5.1, supra.

In a specific embodiment, the viral infection is an enveloped virus infection. In some embodiments, the enveloped virus is a DNA virus. In other embodiments, the enveloped virus is a RNA virus. In some embodiments, the enveloped virus has a double stranded DNA or RNA genome. In other embodiments, the enveloped virus has a single-stranded DNA or RNA genome. In a specific embodiment, the virus infects humans.

In certain embodiments, a compound, a composition comprising a compound, or a combination therapy is administered to a mammal which is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In certain embodiments, a compound, a composition comprising a compound, or a combination therapy is administered to a human at risk for a virus infection. In certain embodiments, a compound, a composition comprising a compound, or a combination therapy is administered to a human with a virus infection. In certain embodiments, the patient is a human 0 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In some embodiments, a compound, a composition comprising a compound, or a combination therapy is administered to a human infant. In other embodiments, a compound, or a combination therapy is administered to a human child. In other embodiments, a compound, a composition comprising a compound, or a combination therapy is administered to a human adult. In yet other embodiments, a compound, a composition comprising a compound, or a combination therapy is administered to an elderly human.

In certain embodiments, a compound, a composition comprising a compound, or a combination therapy is administered to a pet, e.g., a dog or cat. In certain embodiments, a compound, a composition comprising a compound, or a combination therapy is administered to a farm animal or livestock, e.g., pig, cows, horses, chickens, etc. In certain embodiments, a compound, a composition comprising a compound, or a combination therapy is administered to a bird, e.g., ducks or chicken.

In certain embodiments, a compound, a composition comprising a compound, or a combination therapy is administered to a primate, preferably a human, or another mammal, such as a pig, cow, horse, sheep, goat, dog, cat and rodent, in an immunocompromised state or immunosuppressed state or at risk for becoming immunocompromised or immunosuppressed. In certain embodiments, a compound, a composition comprising a compound, or a combination therapy is administered to a subject receiving or recovering from immunosuppressive therapy. In certain embodiments, a compound, a composition comprising a compound, or a combination therapy is administered to a subject that has or is at risk of getting cancer, AIDS, another viral infection, or a bacterial infection. In certain embodiments, a subject that is, will or has undergone surgery, chemotherapy and/or radiation therapy. In certain embodiments, a compound, a composition comprising a compound, or a combination therapy is administered to a subject that has cystic fibrosis, pulmonary fibrosis, or another disease which makes the subject susceptible to a viral infection. In certain embodiments, a compound, a composition comprising a compound, or a combination therapy is administered to a subject that has, will have or had a tissue transplant. In some embodiments, a compound, a composition comprising a compound, or a combination therapy is administered to a subject that lives in a nursing home, a group home (i.e., a home for 10 or more subjects), or a prison. In some embodiments, a compound, a composition comprising a compound, or a combination therapy is administered to a subject that attends school (e.g., elementary school, middle school, junior high school, high school or university) or daycare. In some embodiments, a compound, a composition comprising a compound, or a combination therapy is administered to a subject that works in the healthcare area, such as a doctor or a nurse, or in a hospital. In certain embodiments, a compound, a composition comprising a compound, or a combination therapy is administered to a subject that is pregnant or will become pregnant.

In some embodiments, a patient is administered a compound or a composition comprising a compound, or a combination therapy before any adverse effects or intolerance to therapies other than compounds develops. In some embodiments, compounds or compositions comprising one or more compounds, or combination therapies are administered to refractory patients. In a certain embodiment, refractory patient is a patient refractory to a standard antiviral therapy. In certain embodiments, a patient with a viral infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a viral infection is refractory when viral replication has not decreased or has increased.

In some embodiments, compounds or compositions comprising one or more compounds, or combination therapies are administered to a patient to prevent the onset or reoccurrence of viral infections in a patient at risk of developing such infections. In some embodiments, compounds or compositions comprising one or more compounds, or combination therapies are administered to a patient who are susceptible to adverse reactions to conventional therapies.

In some embodiments, one or more compounds or compositions comprising one or more compounds, or combination therapies are administered to a patient who has proven refractory to therapies other than compounds, but are no longer on these therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring viral infections despite management or treatment with existing therapies.

In some embodiments, the subject being administered one or more compounds or compositions comprising one or more compounds, or combination therapies has not received a therapy prior to the administration of the compounds or compositions or combination therapies. In other embodiments, one or more compounds or compositions comprising one or more compounds, or combination therapies are administered to a subject who has received a therapy prior to administration of one or more compounds or compositions comprising one or more compounds, or combination therapies. In some embodiments, the subject administered a compound or a composition comprising a compound was refractory to a prior therapy or experienced adverse side effects to the prior therapy or the prior therapy was discontinued due to unacceptable levels of toxicity to the subject.

7.2 Mode of Administration

When administered to a patient, a compound is preferably administered as a component of a composition that optionally comprises a pharmaceutically acceptable vehicle. The composition can be administered orally, or by any other convenient route, for example, by infusion or bolus injection, by absorption through epithelial or mucocutaneous linings (e.g., oral mucosa, rectal, and intestinal mucosa) and may be administered together with another biologically active agent. Administration can be systemic or local. Various delivery systems are known, e.g., encapsulation in liposomes, microparticles, microcapsules, capsules, and can be used to administer the compound and pharmaceutically acceptable salts thereof.

Methods of administration include but are not limited to parenteral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a compound into the bloodstream.

In specific embodiments, it may be desirable to administer a compound locally. This may be achieved, for example, and not by way of limitation, by local infusion, topical application, e.g., in conjunction with a wound dressing, by injection, by means of a catheter, by means of a suppository, or by means of an implant, said implant being of a porous, non-porous, or gelatinous material, including membranes, such as sialastic membranes, or fibers. In such instances, administration may selectively target a local tissue without substantial release of a compound into the bloodstream.

In certain embodiments, it may be desirable to introduce a compound into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, a compound is formulated as a suppository, with traditional binders and vehicles such as triglycerides.

For viral infections with cutaneous manifestations, the compound can be administered topically. Similarly, for viral infections with ocular manifestation, the compounds can be administered ocularly.

In another embodiment, a compound is delivered in a vesicle, in particular a liposome (See Langer, 1990, Science 249:1527 1533; Treat et al., in Liposomes in the Therapy of Infectious Disease and Bacterial infection, Lopez-Berestein and Fidler (eds.), Liss, New York, pp. 353 365 (1989); Lopez Berestein, ibid., pp. 317 327; See generally ibid.).

In another embodiment, a compound is delivered in a controlled release system (See, e.g., Goodson, in Medical Applications of Controlled Release, supra, vol. 2, pp. 115 138 (1984)). Examples of controlled-release systems are discussed in the review by Langer, 1990, Science 249:1527 1533 may be used. In one embodiment, a pump may be used (See Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (See Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Pcppas, 1983, J. Macromol. Sci. Rev. Macromol. Chem. 23:61; See also Levy et al., 1985, Science 228:190; During et al., 1989, Ann. Neurol. 25:351; Howard et al., 1989, J. Neurosurg. 71:105). In a specific embodiment, a controlled-release system comprising a compound is placed in close proximity to the tissue infected with a virus to be prevented, treated and/or managed. In accordance with this embodiment, the close proximity of the controlled-release system to the infection may result in only a fraction of the dose of the compound required if it is systemically administered.

In certain embodiments, it may be preferable to administer a compound via the natural route of infection of the virus against which a compound has antiviral activity. For example, it may be desirable to administer a compound of the invention into the lungs by any suitable route to treat or prevent an infection of the respiratory tract by viruses (e.g., influenza virus). Pulmonary administration can also be employed, e.g., by use of an inhaler or nebulizer, and formulation with an aerosolizing agent for use as a spray.

7.3 Agents for Use in Combination with Compounds

Therapeutic or prophylactic agents that can be used in combination with compounds and combinations of compounds for the prevention, treatment and/or management of a viral infection include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such agents include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steriods, and non-steriodal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, gancyclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythomycin, penicillin, mithramycin, and anthramycin (AMC)).

Any therapy which is known to be useful, or which has been used or is currently being used for the prevention, management, and/or treatment of a viral infection or can be used in combination with compounds in accordance with the invention described herein. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996, and Physicians' Desk Reference (61^{st} ed. 1007) for information regarding therapies (e.g., prophylactic or therapeutic agents) which have been or are currently being used for preventing, treating and/or managing viral infections.

7.3.1 Antiviral Agents

Antiviral agents that can be used in combination with the disclosed combinations include, but are not limited to, non-nucleoside reverse transcriptase inhibitors, nucleoside reverse transcriptase inhibitors, protease inhibitors, and fusion inhibitors. In one embodiment, the antiviral agent is selected from the group consisting of amantadine, oseltamivir phosphate, rimantadine, and zanamivir. In another embodiment, the antiviral agent is a non-nucleoside reverse transcriptase inhibitor selected from the group consisting of delavirdine, efavirenz, and nevirapine. In another embodiment, the antiviral agent is a nucleoside reverse transcriptase inhibitor selected from the group consisting of abacavir, didanosine, emtricitabine, emtricitabine, lamivudine, stavudine, tenofovir DF, zalcitabine, and zidovudine. In another embodiment, the antiviral agent is a protease inhibitor selected from the group consisting of amprenavir, atazanavir, fosamprenav, indinavir, lopinavir, nelfinavir, ritonavir, and saquinavir. In another embodiment, the antiviral agent is a fusion inhibitor such as enfuvirtide.

Additional, non-limiting examples of antiviral agents for use in combination compounds include the following: rifampicin, nucleoside reverse transcriptase inhibitors (e.g., AZT, ddl, ddC, 3TC, d4T), non-nucleoside reverse transcriptase inhibitors (e.g., delavirdine efavirenz, nevirapine), protease inhibitors (e.g., aprenavir, indinavir, ritonavir, and saquinavir), idoxuridinc, cidofovir, acyclovir, ganciclovir, zanamivir, amantadinc, and palivizumab. Other examples of anti-viral agents include but are not limited to acemannan; acyclovir; acyclovir sodium; adefovir; alovudine; alvircept sudotox; amantadine hydrochloride (SYMMETREL™); aranotin; arildone; atevirdine mesylate; avridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; kethoxal; lamivudine; lobucavir; memotine hydrochloride; methisazone; nevirapine; oseltamivir phosphate (TAMIFLU™); penciclovir; pirodavir; ribavirin; rimantadinc hydrochloride (FLUMADINE™); saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zanamivir (RELENZA™); zidovudine; and zinviroxime.

7.3.2 Antibacterial Agents

Antibacterial agents, including antibiotics, that can be used in combination with compounds include, but are not limited to, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof. In some embodiments, antibiotics are administered in combination with a compound to prevent and/or treat a bacterial infection.

In a specific embodiment, compounds are used in combination with other protein synthesis inhibitors, including but not limited to, streptomycin, neomycin, erythromycin, carbomycin, and spiramycin.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin. In another embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim.

Additional, non-limiting examples of antibacterial agents for use in combination with compounds include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefrnetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim).

7.4 Dosages & Frequency of Administration

The amount of a compound, or the amount of a composition comprising a compound, that will be effective in the prevention, treatment and/or management of a viral infection can be determined by standard clinical techniques. In vitro or in vivo assays may optionally be employed to help identify optimal dosage ranges. The precise dose to be employed will also depend, e.g., on the route of administration, the combinations with other compounds, the type of invention, and the seriousness of the infection, and should be decided according to the judgment of the practitioner and each patient's or subject's circumstances.

In some embodiments, the dosage of a compound is determined by extrapolating from the no observed adverse effective level (NOAEL), as determined in animal studies. This extrapolated dosage is useful in determining the maximum recommended starting dose for human clinical trials. For instance, the NOAELs can be extrapolated to determine human equivalent dosages (HED). Typically, HED is extrapolated from a non-human animal dosage based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs or minipigs. For a discussion on the use of NOAELs and their extrapolation to determine human equivalent doses, See Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005. In one embodiment, a compound or composition thereof is administered at a dose that is lower than the human equivalent dosage (HED) of the NOAEL over a period of 1 week, 2 weeks, 3 weeks, 1 month, 2 months, three months, four months, six months, nine months, 1 year, 2 years, 3 years, 4 years or more.

In certain embodiments, a dosage regime for a human subject can be extrapolated from animal model studies using the dose at which 10% of the animals die (LD10). In general the starting dose of a Phase I clinical trial is based on preclinical testing. A standard measure of toxicity of a drug in preclinical testing is the percentage of animals that die because of treatment. It is well within the skill of the art to correlate the LD10 in an animal study with the maximal-tolerated dose (MTD) in humans, adjusted for body surface area, as a basis to extrapolate a starting human dose. In some embodiments, the interrelationship of dosages for one animal model can be converted for use in another animal, including humans, using conversion factors (based on milligrams per meter squared of body surface) as described, e.g., in Freireich et al., Cancer Chemother. Rep., 1966, 50:219-244. Body surface area may be approximately determined from height and weight of the patient. See, e.g., Scientific Tables, Geigy Pharmaceuticals, Ardley, N. Y., 1970, 537. In certain embodiments, the adjustment for body surface area includes host factors such as, for example, surface area, weight, metabolism, tissue distribution, absorption rate, and excretion rate. In addition, the route of administration, excipient usage, and the specific disease or virus to target are also factors to consider. In one embodiment, the standard conservative starting dose is about 1/10 the murine LD10, although it may be even lower if other species (i.e., dogs) were more sensitive to the compound. In other embodiments, the standard conservative starting dose is about 1/100, 1/95, 1/90, 1/85, 1/80, 1/75, 1/70, 1/65, 1/60, 1/55, 1/50, 1/45, 1/40, 1/35, 1/30, 1/25, 1/20, 1/15, 2/10, 3/10, 4/10, or 5/10 of the murine LD10. In other embodiments, an starting dose amount of a compound in a human is lower than the dose extrapolated from animal model studies. In another embodiment, an starting dose amount of a compound in a human is higher than the dose extrapolated from animal model studies. It is well within the skill of the art to start doses of the active composition at relatively low levels, and increase or decrease the dosage as necessary to achieve the desired effect with minimal toxicity.

Exemplary doses of compounds or compositions include milligram or microgram amounts per kilogram of subject or sample weight (e.g., about 1 microgram per kilogram to about 500 milligrams per kilogram, about 5 micrograms per kilogram to about 100 milligrams per kilogram, or about 1 microgram per kilogram to about 50 micrograms per kilogram). In specific embodiments, a daily dose is at least 50 mg, 75 mg, 100 mg, 150 mg, 250 mg, 500 mg, 750 mg, or at least 1 g.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM. In a specific embodiment, the dosage is 0.25 μg/kg or more, preferably 0.5 μg/kg or more, 1 μg/kg or more, 2 μg/kg or more, 3 μg/kg or more, 4 μg/kg or more, 5 μg/kg or more, 6 μg/kg or more, 7 μg/kg or more, 8 μg/kg or more, 9 μg/kg or more, or 10 μg/kg or more, 25 μg/kg or more, preferably 50 μg/kg or more, 100 μg/kg or more, 250 μg/kg or more, 500 μg/kg or more, 1 mg/kg or more, 5 mg/kg or more, 6 mg/kg or more, 7 mg/kg or more, 8 mg/kg or more, 9 mg/kg or more, or 10 mg/kg or more of a patient's body weight.

In another embodiment, the dosage is a unit dose of 5 mg, preferably 10 mg, 50 mg, 100 mg, 150 mg, 200 mg, 250 mg, 300 mg, 350 mg, 400 mg, 500 mg, 550 mg, 600 mg, 650 mg, 700 mg, 750 mg, 800 mg or more. In another embodiment, the dosage is a unit dose that ranges from about 5 mg to about 100 mg, about 100 mg to about 200 μg, about 150 mg to about 300 mg, about 150 mg to about 400 mg, 250 μg to about 500 mg, about 500 mg to about 800 mg, about 500 mg to about 1000 mg, or about 5 mg to about 1000 mg.

In certain embodiments, suitable dosage ranges for oral administration are about 0.001 milligram to about 500 milligrams of a compound, per kilogram body weight per day. In specific embodiments of the invention, the oral dose is about 0.01 milligram to about 100 milligrams per kilogram body weight per day, about 0.1 milligram to about 75 milligrams per kilogram body weight per day or about 0.5 milligram to 5 milligrams per kilogram body weight per day. The dosage amounts described herein refer to total amounts administered; that is, if more than one compound is administered, then, in some embodiments, the dosages correspond to the total amount administered. In a specific embodiment, oral compositions contain about 10% to about 95% a compound of the invention by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. In some embodiments, suitable dosage ranges for intranasal administration are about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 500 milligrams per kilogram of body weight per day. Suitable doses for topical administration include doses that are in the range of about 0.001 milligram to about 50 milligrams, depending on the area of administration. Effective doses may be extrapolated from dose-response curves derived from in vitro or animal model test systems. Such animal models and systems are well known in the art.

A person skilled in the art may also determine the early viral response (EVR) and sustained viral response (SVR) to determine which dose of a particular combination is most appropriate in a particular case. Sustained viral response (SVR) is considered to be the defining indicator of successful treatment of a viral disease, including hepatitis C. A SVR is commonly understood to mean the absence of virus in the patient's serum six months after treatment was stopped. Early viral response (EVR) is commonly understood to mean a minimum decrease of 2 log₁₀ in the viral load (commonly determined by measuring the presence in the serum of viral DNA or RNA) during the first 12 weeks of treatment.

In another embodiment, a subject is administered one or more doses of a prophylactically or therapeutically effective amount of a compound, or a combination of two or more compounds, wherein the prophylactically or therapeutically effective amount is not the same for each dose. In another embodiment, a subject is administered one or more doses of a prophylactically or therapeutically effective amount of a compound or a combination, wherein the dose of a prophylactically or therapeutically effective amount of one or more of the compounds administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses. In another embodiment, a subject is administered one or more doses of a prophylactically or therapeutically effective amount of a compound or combinations of compounds described herein may involve administering to the subject of two or more of the compounds in the same dosage form, wherein the dose of one or more of the compounds is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses.

In certain embodiments, a subject is administered a compound or a composition in an amount effective to inhibit or reduce viral genome replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a compound or a composition in an amount effective to inhibit or reduce viral genome replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a compound or a composition in an amount effective to inhibit or reduce viral genome replication by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or other known to one of skill in the art.

In certain embodiments, a subject is administered a compound or a composition in an amount effective to inhibit or reduce viral protein synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a compound or a composition in an amount effective to inhibit or reduce viral protein synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a compound or a composition in an amount effective to inhibit or reduce viral protein synthesis by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In certain embodiments, a subject is administered a compound or a composition in an amount effective to inhibit or reduce viral infection by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, a subject is administered a compound or a composition in an amount effective to inhibit or reduce viral infection by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In certain embodiments, a subject is administered a compound or a composition in an amount effective to inhibit or reduce viral replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, a subject is administered a compound or a composition in an amount effective to inhibit or reduce viral replication by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art. In other embodiments, a subject is administered a compound or a composition in an amount effective to inhibit or reduce viral replication by 1 log, 1.5 logs, 2 logs, 2.5 logs, 3 logs, 3.5 logs, 4 logs, 5 logs or more relative to a negative control as determined using an assay described herein or others known to one of skill in the art.

In certain embodiments, a subject is administered a compound or a composition in an amount effective to inhibit or reduce the ability of the virus to spread to other individuals by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a compound or a composition in an amount effective to inhibit or reduce the ability of the virus to spread to other cells, tissues or organs in the subject by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art.

In certain embodiments, a subject is administered a compound or a composition in an amount effective to inhibit or reduce viral induced lipid synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a compound or a composition in an amount effective to inhibit or reduce viral induced lipid synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a compound or a composition in an amount effective to inhibit or reduce viral induced lipid synthesis by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In certain embodiments, a dose of a compound or a composition is administered to a subject every day, every other day, every couple of days, every third day, once a week, twice a week, three times a week, or once every two weeks. In other embodiments, two, three or four doses of a compound or a composition is administered to a subject every day, every couple of days, every third day, once a week or once every two weeks. In some embodiments, a dose(s) of a compound or a composition is administered for 2 days, 3 days, 5 days, 7 days, 14 days, or 21 days. In certain embodiments, a dose of a compound or a composition is administered for 1 month, 1.5 months, 2 months, 2.5 months, 3 months, 4 months, 5 months, 6 months or more.

The dosages of prophylactic or therapeutic agents which have been or are currently used for the prevention, treatment and/or management of a viral infection can be determined using references available to a clinician such as, e.g., the Physicians' Desk Reference (61^{st} ed. 2007). Preferably, dosages lower than those which have been or are currently being used to prevent, treat and/or manage the infection are utilized in combination with one or more compounds or compositions.

For compounds which have been approved for uses other than prevention, treatment or management of viral infections, safe ranges of doses can be readily determined using references available to clinicians, such as e.g., the Physician's Desk Reference (61^{st} ed. 2007).

The above-described administration schedules are provided for illustrative purposes only and should not be considered limiting. A person of ordinary skill in the art will readily understand that all doses are within the scope of the invention.

It is to be understood and expected that variations in the principles of invention herein disclosed may be made by one skilled in the art and it is intended that such modifications are to be included within the scope of the present invention.

Throughout this application, various publications are referenced. These publications are hereby incorporated into this application by reference in their entireties to more fully describe the state of the art to which this invention pertains. The following examples further illustrate the invention, but should not be construed to limit the scope of the invention in any way.

### Example 1

### Enhanced Antiviral Effects of a Combination of an ACC Inhibitor and an HCV Protease Inhibitor

HCV encoded proteolytic activity is required for infection and replication. The present example concerns the combined use of an ACC inhibitors (e.g., TOFA) and an HCV protease inihibitor (e.g., boceprevir) to antagonize viral replication. In each assay, various concentrations of TOFA are combined with various concentrations of boceprevir and cell cultures exposed to HCV are assayed for virus replication. In one series of tested combinations, a physiological concentration of boceprivir is held constant as the dose of TOFA is increased. Control cultures are treated with no drug, boceprivir alone or the various concentrations of TOFA alone. Samples are taken at 24, 48, 72 and 96 hours after initiation of drug treatment. The antiviral effect of boceprivir plus each concentration of TOFA is then compared to the activity of boceprivir alone or the various concentrations of TOFA alone. The relative toxicity of the different combinations is also assayed.

In the presence of a pharmacologically acceptable concentration of boceprivir, the concentration of TOFA required to produce a 10-fold reduction in HCV replication is markedly reduced. For a given TOFA concentration, the magnitude of the therapeutic effect is increased when boceprivir is also present. A similar effect is observed when the TOFA dose is held constant and the concentration of boceprivir is varied.

For a given reduction in HCV replication, host cell toxicity is reduced when both drugs are used in combination.

1. A method of treating or preventing HCV infection comprising administering to a subject in need thereof a therapeutically effective amount of (i) a compound that is an inhibitor of acetyl-CoA carboxylase (ACC) or a prodrug thereof, or pharmaceutically acceptable salt or ester of said compound or prodrug and (ii) a compound that is a modulator of an HCV-associated component or a prodrug thereof, or pharmaceutically acceptable salt or ester of said compound or prodrug.

2. The method of claim 1, wherein the inhibitor of ACC inhibits ACC1, ACC2, or both ACC1 and ACC2.

3. The method of claim 1, wherein the ACC inhibitor is a compound of formula XII: 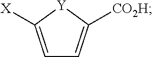
wherein:
X is —(C₅-C₂₀)alkyl, —O(C₅-C₂₀)alkyl, —(C₅-C₂₀)haloalkyl, —O(C₅-C₂₀)haloalkyl, -halo, —OH, —(C₅-C₂₀)alkenyl, —(C₅-C₂₀)alkynyl, —(C₅-C₂₀)alkoxy-alkenyl, —(C₅-C₂₀)hydroxyalkyl, —O(C₁-C₆)alkyl, —CO₂(C₁-C₆)alkyl, —O(C₅-C₂₀)alkenyl, —O(C₅-C₂₀)alkynyl, —O(C₅-C₂₀)cycloalkyl, —S(C₅-C₂₀)alkyl, —NH(C₅-C₂₀)alkyl, —NHCO(C₅-C₂₀)alkyl, —N(C₁-C₆)alkylCO(C₅-C₂₀)alkyl or —O(C₅-C₂₀)alkoxy; and
Y is O, S, —NH or N(C₁-C₆)alkyl.

4. The method of claim 3, wherein the ACC inhibitor is TOFA.

5. The method of claim 1, wherein the ACC inhibitor is a compound of formula XIII: 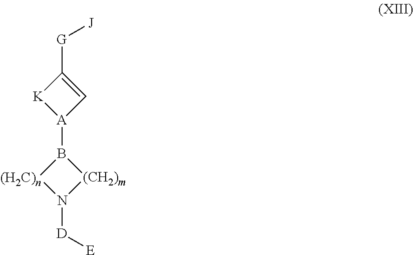
wherein A-B is N—CH or CH—N; K is (CH₂)ᵣ wherein r is 2, 3 or 4; m and n are each independently 1, 2 or 3 when A-B is N—CH or m and n are each independently 2 or 3 when A-B is CH—N; the dashed line represents the presence of an optional double bond;
D is carbonyl or sulfonyl;
E is either a) a bicyclic ring consisting of two fused fully unsaturated five to seven membered rings, taken independently, each of said rings optionally having one to four heteroatoms selected independently from oxygen, sulfur and nitrogen, or b) a tricyclic ring consisting of two fused fully unsaturated five to seven membered rings, taken independently, each of said rings optionally having one to four heteroatoms selected independently from oxygen, sulfur and nitrogen, said two fused rings fused to a third partially saturated, fully unsaturated or fully saturated five to seven membered ring, said third ring optionally having one to four heteroatoms selected independently from oxygen, sulfur and nitrogen; or c) a tetracyclic ring comprising a bicyclic ring consisting of two fused fully unsaturated five to seven membered rings, taken independently, each of said rings optionally having one to four heteroatoms selected independently from oxygen, sulfur and nitrogen, said bicyclic ring fused to two fully saturated, partially saturated or fully unsaturated five to seven membered monocyclic rings taken independently, each of said rings optionally having one to four heteroatoms selected independently from oxygen, sulfur and nitrogen or said bicyclic ring fused to a second bicyclic ring consisting of two fused fully saturated, partially saturated or fully unsaturated five to seven membered rings, taken independently, each of said rings optionally having one to four heteroatoms selected independently from oxygen, sulfur and nitrogen; or d) a teraryl ring comprising a fully unsaturated five to seven membered ring, said ring optionally having one to four heteroatoms selected independently from oxygen, sulfur and nitrogen, and said ring di-substituted independently with a fully unsaturated five to seven membered ring to form a teraryl nonfused ring system, each of said substituent rings optionally having one to four heteroatoms selected independently from oxygen, sulfur and nitrogen, wherein said E bi-, tri- or tetra cyclic ring or teraryl ring is optionally mono-, di- or tri-substituted independently on each ring used to form the bi-, tri- or tetra cyclic ring or teraryl ring with halo, hydroxy, amino, cyano, nitro, oxo, carboxy, (C₁-C₆) alkyl, (C₂-C₆) alkenyl, (C₂-C₆) alkynyl, (C₁-C₆) alkoxy, (C₁-C₄) alkylthio, (C₁-C₆) alkoxycarbonyl;
wherein said E bi-, tri- or tetra-cyclic ring or teraryl ring is optionally mono-substituted with a partially saturated, fully saturated or fully unsaturated three to eight membered ring R₁₀ optionally having one to four heteroatoms selected independently from oxygen, sulfur and nitrogen or a bicyclic ring R″ consisting of two fused partially saturated, fully saturated or fully unsaturated three to eight membered rings, taken independently, each of said rings optionally having one to four heteroatoms selected independently from oxygen, sulfur and nitrogen, said R₁₀ and R″ rings optionally additionally bridged and said R₁₀ and R″ rings optionally linked through a fully saturated, partially unsaturated or fully unsaturated one to four membered straight or branched carbon chain wherein the carbon (s) may optionally be replaced with one or two heteroatoms selected independently from oxygen, nitrogen and sulfur, provided said E bicyclic ring has at least one substituent and the E bicyclic ring atom bonded to D is carbon; wherein said R₁₀ or R″ring is optionally mono-, di- or tri-substituted independently with halo, hydroxy, amino, cyano, nitro, oxo, carboxy, (C₁-C₆) alkyl, (C₂-C₆) alkenyl, (C₂-C₆) alkynyl, (C₁-C₆) alkoxy, (C₁-C₄)alkylthio, (C₁-C₆) alkoxycarbonyl, (C₁-C₆) alkylcarbonyl, (C₁-C₆) alkylcarbonylamino, or mono-N— or di-N,N—(C₁-C₆) alkylamino or mono-N— or di-N,N—(C₁-C₆) alkylaminocarbonyl wherein said (C₁-C₆) alkyl and (C₁-C₆) alkoxy substituents are also optionally mono-, di- or tri-substituted independently with halo, hydroxy, (C₁-C₆) alkoxy, amino, mono-N— or di-N,N—(C₁-C₆) alkylamino or from one to nine fluorines;
G is carbonyl, sulfonyl or CR₇R₈; wherein R₇ and R₈ are each independently H, (C₁-C₆) alkyl, (C₂-C₆) alkenyl or (C₂-C₆) alkynyl or a five to seven membered partially saturated, fully saturated or fully unsaturated ring optionally having one heteroatom selected from oxygen, sulfur and nitrogen;
J is OR′, NR₂R₃ or CR₄R₅R₆; wherein R′, R₂ and R₃ are each independently H, Q, or a (C₁-C₁₀) alkyl, (C₃-C₁₀) alkenyl or (C₃-C₁₀) alkynyl substituent wherein said carbon(s) may optionally be replaced with one or two heteroatoms selected independently from oxygen, nitrogen and sulfur and wherein said sulfur is optionally mono- or di-substituted with oxo, said carbon (s) is optionally mono-substituted with oxo, said nitrogen is optionally di-substituted with oxo, said carbon (s) is optionally mono-, di- or tri-substituted independently with halo, hydroxy, amino, nitro, cyano, carboxy, (C₁-C₄) alkylthio, (C₁-C₆)alkyloxycarbonyl, mono-N— or di-N,N—(C₁-C₆) alkylamino or mono-N— or di-N,N—(C₁-C₆)alkylaminocarbonyl; and said chain is optionally mono-substituted with Q₁; wherein Q and Q₁ are each independently a partially saturated, fully saturated or fully unsaturated three to eight membered ring optionally having one to three heteroatoms selected independently from oxygen, sulfur and nitrogen or a bicyclic ring consisting of two fused or spirocyclic partially saturated, fully saturated or fully unsaturated three to six membered rings, taken independently, said bicyclic ring optionally having one to three heteroatoms selected independently from oxygen, sulfur and nitrogen, said mono or bicyclic ring optionally additionally bridged with (C₁-C₃) alkylen wherein said (C₁-C₃) alkylen carbons are optionally replaced with one to two heteroatoms selected independently from oxygen, sulfur and nitrogen; wherein said Q and Q₁ ring are each independently optionally mono-, di-, tri-, or tetra-substituted independently with halo, hydroxy, amino, nitro, cyano, oxo, carboxy, (C₁-C₆)alkyl, (C₂-C₆) alkenyl, (C₂-C₆) alkynyl, (C₁-C₆) alkoxy, (C₁-C₄) alkylthio, (C₁-C₆) alkylcarbonyl, (C₁-C₆) alkylcarbonylamino, (C₁-C₆)alkyloxycarbonyl, mono-N— or di-N,N—(C₁-C₆) alkylamino, mono-N— or di-N,N—(C₁-C₆)alkylaminosulfonyl, mono-N— or di-N,N—(C₁-C₆) alkylaminocarbonyl, wherein said (C₁-C₆) alkyl substituent is optionally mono-, di- or tri-substituted independently with halo, hydroxy, amino, nitro, cyano, oxo, carboxy, (C₁-C₆)alkoxy, (C₁-C₄) alkylthio, (C₁-C₆)alkyloxycarbonyl or mono-N— or di-N,N—(C₁-C₆)alkylamino wherein said (C₁-C₆) alkyl substituent is also optionally substituted with from one to nine fluorines;
or wherein R₂ and R₃ can be taken together with the nitrogen atom to which they are attached to form a partially saturated, fully saturated or fully unsaturated three to eight membered ring optionally having one to three additional heteroatoms selected independently from oxygen, sulfur and nitrogen or a bicyclic ring consisting of two fused, bridged or spirocyclic partially saturated, fully saturated or fully unsaturated three to six membered rings, taken independently, said bicyclic ring optionally having one to three additional heteroatoms selected independently from oxygen, sulfur and nitrogen or a tricyclic ring consisting of three fused, bridged or spirocyclic partially saturated, fully saturated or fully unsaturated three to six membered rings, taken independently, said tricyclic ring optionally having one to three additional heteroatoms selected independently from oxygen, sulfur and nitrogen; wherein said NR₂R₃ ring is optionally mono-, di-, tri- or tetra-substituted independently with R15, halo, hydroxy, amino, nitro, cyano, oxo, carboxy, (C₁-C₆) alkyl, (C₂-C₆) alkenyl, (C₂-C₆) alkynyl, (C₁-C₆) alkoxy, (C₁-C₄) alkylthio, (C₁-C₆) alkylcarbonylamino or mono-N— or di-N,N—(C₁-C₆) alkylamino, wherein said (C₁-C₆) alkyl substituent is optionally mono-, di- or tri-substituted independently with halo, hydroxy, amino, nitro, cyano, oxo, carboxy, (C₁-C₆) alkoxy, (C₁-C₄) alkylthio, (C₁-C₆) alkyloxycarbonyl, mono-N— or di-N,N—(C₁-C₆) alkylamino, said (C₁-C₆) alkyl substituent is also optionally substituted with from one to nine fluorines;
wherein three heteroatoms selected independently from oxygen, sulfur and nitrogen wherein said ring is optionally mono-, di- or tri-substituted with halo, hydroxy, amino, nitro, cyano, oxo, carboxy, (C₁-C₆) alkyl, (C₂-C₆) alkenyl, (C₂-C₆) alkynyl, (C₁-C₄)alkylthio, (C₁-C₆) alkoxy, (C₁-C₆)alkylcarbonylamino, mono-N— or di-N,N—(C₁-C₆) alkylamino; wherein said NR₂R₃ ring is optionally substituted with a partially saturated, fully saturated or fully unsaturated three to eight membered ring optionally having one to three heteroatoms selected independently from oxygen, sulfur and nitrogen or a bicyclic ring consisting of two fused partially saturated, fully saturated or fully unsaturated three to six membered rings, taken independently, said bicyclic ring optionally having one to three heteroatoms selected independently from oxygen, sulfur and nitrogen, said mono or bicyclic ring optionally additionally bridged said ring optionally having one to three heteroatoms selected independently from oxygen, sulfur and nitrogen, wherein said (C₁-C₆) alkyl and said ring are optionally mono-, di- or tri-substituted with halo, hydroxy, amino, nitro, cyano, oxo, carboxy, (C₂-C₆) alkenyl, (C₃-C₆) alkynyl, (C₁-C₆) alkylcarbonylamino, hydroxy, (C₁-C₆) alkoxy, (C₁-C₄) alkylthio, (C₁-C₆) alkoxy, mono-N— or di-N,N—(C₁-C₆) alkylamino; wherein R₄, R₅ and R₆ are independently H, halo, hydroxy, (C₁-C₆) alkyl or R₄ and R₅ are taken together to form a partially saturated, fully saturated or fully unsaturated three to eight membered ring, said ring optionally having one to three heteroatoms selected independently from oxygen, sulfur and nitrogen, wherein said (C₁-C₆) alkyl and said ring are optionally mono-, di- or tri-substituted with halo, hydroxy, amino, nitro, cyano, oxo, carboxy, (C₂-C₆) alkenyl, (C₃-C₆) alkynyl, (C₁-C₆) alkylcarbonylamino, hydroxy, (C₁-C₆) alkoxy, (C₁-C₄) alkylthio, (C₁-C₆) alkoxy, mono-N— or di-N,N—(C₁-C₆) alkylamino with the proviso that 1′-(anthracene-9-carbonyl)-[1,4′]bipiperidinyl-3-carboxylic aciddiethyiamide; 1′-(1-oxa-2,3-diaza-cyclopenta[a]naphthalene-5-sulfonyl)-[1,4′]bipiperidinyl-3 carboxylic acid diethylamide; 1′-(5-dimethylamino-naphthalene-1-sulfonyl)-[1,4′]bipiperidinyl-3-carboxylic acid diethylamide; 1′-(9,10,10-trioxo-9,10-dihydro-thioxanthene-3-carbonyl)-[1-4′]bipiperidinyl-3-carboxylic acid diethylamide; and 1′-(2-Oxo-2H-chromen-3-carbonyl)-[1,4′]bipiperidinyl-3-carboxylic acid diethylamide are not included.

6. The method of claim 5, wherein the ACC inhibitor is CP-610431.

7. The method of claim 5, wherein the ACC inhibitor is CP-640186.

8. The method of claim 1, wherein an immunomodulator is also administered to the subject.

9. The method of claim 8, wherein the immunomodulator is one or more of Pegasys, Roferon-A, Pegintron, Intron A, Albumin IFN-α, locteron, Peginterferon-λ, omega-IFN, medusa-IFN, belerofon, infradure, Interferon alfacon-1, and Veldona.

10. The method of claim 1, wherein one or more of ribavirin or a ribavirin analog selected from taribavirin, mizoribine, merimepodib, mycophenolate mofetil, and mycophenolate is also administered to the subject.

11. A method of treating or preventing HCV infection comprising administering to a subject in need thereof a therapeutically effective amount of (i) a compound that is a modulator of a host cell target or a prodrug thereof, or pharmaceutically acceptable salt or ester of said compound or prodrug and (ii) a compound that is a modulator of an HCV-associated component or a prodrug thereof, or pharmaceutically acceptable salt or ester of said compound or prodrug.

12. The method of claim 11, wherein an immunomodulator is also administered to the subject.

13. The method of claim 12, wherein the immunomodulator is one or more of Pegasys, Roferon-A, Pegintron, Intron A, Albumin IFN-α, locteron, Peginterferon-λ, omega-IFN, medusa-IFN, belerofon, infradure, Interferon alfacon-1, and Veldona.

14. The method of claim 11, wherein one or more of ribavirin or a ribavirin analog selected from taribavirin, mizoribine, merimepodib, mycophenolate mofetil, and mycophenolate is also administered to the subject.

15. The method of claim 11, wherein the compound that is a modulator of a host cell target is an inhibitor of an acyl-CoA:cholesterol acyl-transferase (ACAT).

16. The method of claim 15 wherein the inhibitor of ACAT inhibits ACAT1, ACAT2, or both ACAT1 and ACAT2.

17. The method of claim 15, wherein the ACAT inhibitor is pactimibe, Compound 1, Compound 21, Compound 12g, SMP-797, CL-283,546, Wu-V-23 or eflucimibe.

18. The method of claim 15 wherein the inhibitor of ACAT is a compound of formula V: 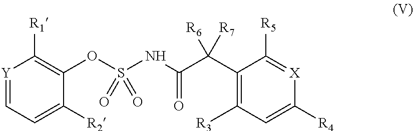
wherein
X and Y are independently selected from N and CH;
R_{1′} and R_{2′} are independently selected from H, C₁₋₆ alkyl which may be optionally substituted with F, OCH₃ and OH, and C₁₋₆ cycloalkyl;
R6 and R7 are independently selected from H, and C₁₋₃ alkyl, or R₆ and R₇ taken together may form a C₃₋₆ cycloalkyl;
R₃, R₄ and R₅ are independently selected from H, C1-6 alkyl which may be optionally substituted with F, OCH₃ and OH, and C₁₋₆ cycloalkyl;
additionally or alternatively, one of R₆ or R₇ may be taken together with R₅ to form a C₅₋₁₁ cycloalkyl ring.

19. The method of claim 18, wherein the compound is avasimibe.

20. (canceled)

21. (canceled)

22. (canceled)

23. The method of claim 11, wherein the compound that is a modulator of a host cell target is an inhibitor of a long-chain acyl-CoA synthetase (ACSL).

24. The method of claim 23, wherein the ACSL inhibitor is a compound of formula I: 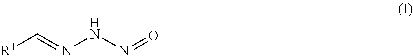
wherein R¹ is a carbon chain having from 3 to 23 atoms and heteroatoms;
wherein the carbon chain comprises 0-10 double bonds and 0-4 heteroatoms; and
wherein 0-8 of the carbon atoms of R¹ are optionally substituted.

25. The method of claim 23, wherein the ACSL inhibitor is triacsin C.

26. (canceled)

27. (canceled)

28. (canceled)

29. The method of claim 11, wherein the compound that is a modulator of a host cell target is an inhibitor of an elongase (ELOVL).

30. The method of claim 29, wherein the inhibitor of an elongase is an inhibitor of one or more of ELOVL2, ELOVL3, ELOVL6.

31. (canceled)

32. (canceled)

33. (canceled)

34. The method of claim 11, wherein the compound that is a modulator of a host cell target is an inhibitor of fatty acid synthase (FAS).

35. The method of claim 34, wherein the inhibitor of fatty acid synthase is C75 or orlistat.

36. (canceled)

37. (canceled)

38. (canceled)

39. The method of claim 11, wherein the compound that is a modulator of a host cell target is an inhibitor of HMG-CoA reductase.

40. The method of claim 39, wherein the HMG-CoA reductase inhibitor is fluvastatin, lovastatin, mevastatin, lovastatin, pravastatin, simvastatin, atorvastatin, itavastatin, or visastatin.

41. (canceled)

42. (canceled)

43. (canceled)

44. The method of claim 11, wherein the compound that is a modulator of a host cell target is an inhibitor of lipid droplet formation.

45. The method of claim 44, wherein the inhibitors of lipid droplet accumulation is PF-1052, spylidone, sespendole, terpendole C, rubimaillin, Compound 7, Compound 8, Compound 9, vermisporin; beauveriolides; phenochalasins; isobisvertinol; or K97-0239.

46. (canceled)

47. (canceled)

48. (canceled)

49. The method of claim 11, wherein the compound that is a modulator of a host cell target an inhibitor of serine palmitoyl transferase (SPT).

50. The method of claim 49, wherein the inhibitor of SPT is myriocin, sphingofungin B, sphingofungin C, sphingofungin E sphingofungin F, lipoxamycin, viridiofungin A, sulfamisterin, or NA255.

51. (canceled)

52. (canceled)

53. (canceled)

54. The method of claim 1, wherein the modulator of an HCV-associated component is an HCV protease inhibitor.

55. The method of claim 54, wherein the HCV protease inhibitor is selected from boceprevir, telaprevir, ITMN-191, SCH-900518, TMC-435, BI-201335, MK-7009, VX-500, VX-813, BMS650032, VBY376, R7227, VX-985, ABT-333, ACH-1625, ACH-2684, GS-9256, GS-9451, MK-5172, and ABT-450.

56. The method of claim 54, wherein the HCV protease inhibitor is boceprevir or telaprevir.

57. The method of claim 1, wherein the modulator of an HCV-associated component is an HCV helicase (NS3) inhibitor.

58. The method of claim 57, wherein the modulator of an HCV-associated component is an HCV helicase (NS3) inhibitor selected from compounds of the following structure 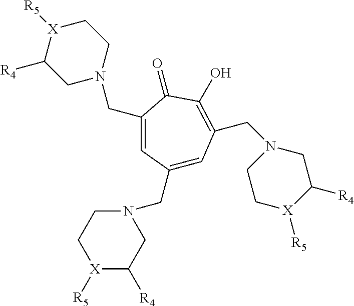
wherein X is N, R₄ is H and R₅ is CH₃; X is CH, R₄ is H and R₅ is CH₃; or X is CH, R₄ is CH₃ and R₅ is H.

59. The method of claim 57, wherein the modulator of an HCV-associated component is an HCV helicase (NS3) inhibitor selected from

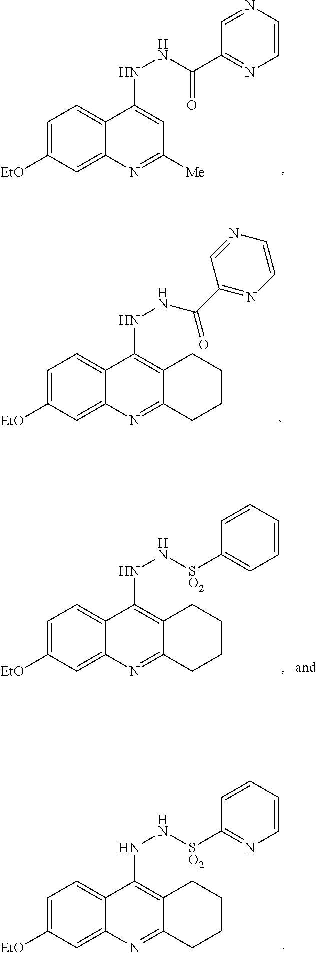 [SMILES: CCOc1ccc2c(NNC(=O)c3cnccn3)cc(C)nc2c1 ; CCOc1ccc2c(NNC(=O)c3cnccn3)c3c(nc2c1)CCCC3 ; CCOc1ccc2c(NNS(=O)(=O)c3ccccc3)c3c(nc2c1)CCCC3 ; CCOc1ccc2c(NNS(=O)(=O)c3ccccn3)c3c(nc2c1)CCCC3]

60. The method of claim 57, wherein the modulator of an HCV-associated component is an HCV helicase (NS3) inhibitor selected from

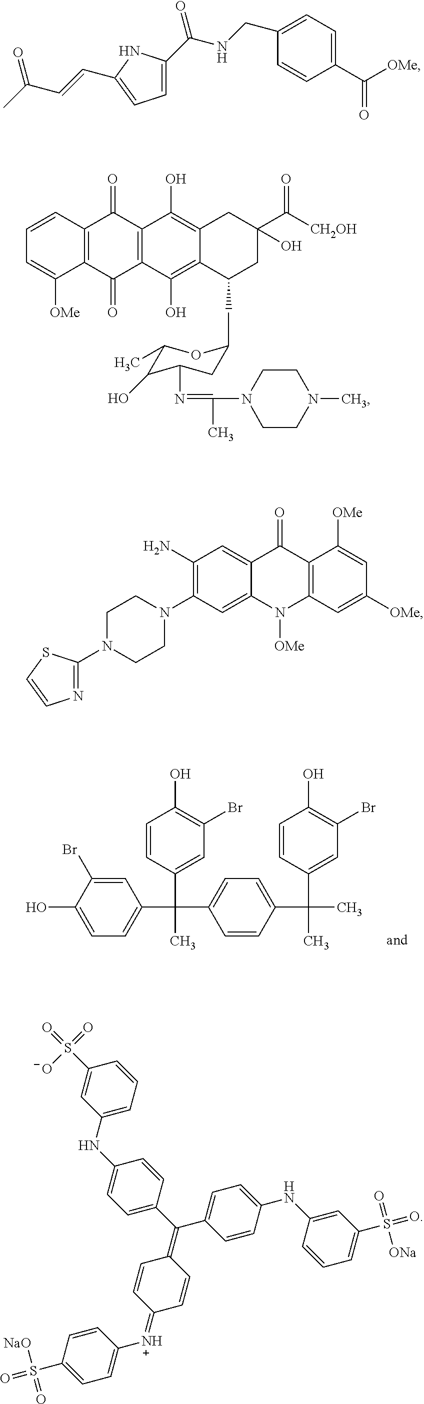

61. The method of claim 1, wherein the modulator of an HCV-associated component is an inhibitor HCV nonstructural protein 4B (NS4B).

62. The method of claim 61, wherein the inhibitor of NS4B is GSK-8853, clemizole, a benzimidazole RBI (B-RBI) or an indazole RBI (I-RBI).

63. The method of claim 1, wherein the modulator of an HCV-associated component is an inhibitor HCV nonstructural protein 5A (NS5A)

64. The method of claim 63, wherein the inhibitor of NS5A is BMS-790052, A-689, A-831, EDP239, GS5885, GSK805, PPI-461 BMS-824393 or ABT-267.

65. The method of claim 1, wherein the modulator of an HCV-associated component is an inhibitor of HCV polymerase (NS5B).

66. The method of claim 65, wherein the inhibitor of NS5B is a nucleoside analog, a nucleotide analog, or a non-nucleoside inhibitor.

67. The method of claim 65, wherein the inhibitor of NS5B is valopicitabine, R1479, R1626, R7128, RG7128, TMC649128, IDX184, PSI-352938, INX-08189, GS6620, filibuvir, HCV-796, VCH-759, VCH-916, ANA598, VCH-222 (VX-222), BI-207127, MK-3281, ABT-072, ABT-333, GS9190, BMS791325, GSK2485852A, PSI-7851, PSI-7976, and PSI-7977.

68. The method of claim 1, wherein the modulator of an HCV-associated component is an inhibitor of HCV viral ion channel forming protein (p7).

69. The method of claim 69, wherein the inhibitor of p7 is BIT225 or HPH116.

70. The method of claim 1, wherein the modulator of an HCV-associated component is an IRES inhibitor.

71. The method of claim 70, wherein the IRES inhibitor is Mifepristone, Hepazyme, ISIS14803, and siRNAs/shRNAs.

72. The method of claim 1, wherein the modulator of an HCV-associated component is an HCV entry inhibitor.

73. The method of claim 72, wherein the HCV entry inhibitor is HuMax HepC, JTK-652, PRO206, SP-30, or ITX5061.

74. The method of claim 1, wherein the modulator of an HCV-associated component is a cyclosporin inhibitor.

75. The method of claim 74, wherein the cyclophilin inhibitor is Debio 025, NIM811, SCY-635, or cyclosporin-A.

76. The method of claim 1, wherein the modulator of an HCV-associated component is modulator of microRNA-122 (miR-122).

77. The method of claim 76 wherein the modulator of microRNA-122 is SPC3649.
